(19) United States
(12) Patent Application Publication
(10) Pub. No.: US 2004/0086479 A1
(43) Pub. Date: May 6, 2004

(54) Novel dendritic polymers, crosslinked gels, and their biomedical uses

(75) Inventors: Mark W. Grinstaff, Durham, NC (US); Michael A. Carnahan, Durham, NC (US); Terry Kim, Durham, NC (US); Nate Luman, Durham, NC (US); Meredith Morgan, Durham, NC (US); Michel Wathier, Durham, NC (US)

Correspondence Address: NIXON & VANDERHYE, PC, 1100 N GLEBE ROAD, 8TH FLOOR, ARLINGTON, VA, 22201-4714, US

(73) Assignee: DUKE UNIVERSITY, Durham, NC

(21) Appl. No.: 10/423,053

(22) Filed: Apr. 25, 2003

Related U.S. Application Data
(63) Continuation-in-part of application No. PCT/US02/05638, filed on Feb. 26, 2002.
(60) Provisional application No. 60/270,881, filed on Feb. 26, 2001.

(51) Int. Cl. A61K 31/74

(52) U.S. Cl. 424/78.17; 525/54.1

(57) ABSTRACT

Crosslinkable polymers, such as dendritic macromolecules and their in vitro, in vivo, and in situ uses are disclosed. These biomaterials/polymers are likely to be an effective sealant/glue for a variety of surgical procedures where the site of the wound is not easily accessible or when sutureless surgery is desirable. Crosslinkable dendritic macromolecules can be fabricated into cell scaffold/gel/matrix of specified shapes and sizes using chemical techniques. The polymers, after being crosslinked, can be seeded with cells and then used to repair or replace organs, tissue, or bones. Alternatively, the polymers and cells can be mixed and then injected into the in vivo site and crosslinked in situ for organ, tissue, or bone repair or replacement. The crosslinked polymers provide three dimensional templates for new cell growth that is suitable for a variety of reconstructive procedures, including custom molding of cell implants to reconstruct three dimensional tissue defects. The crosslinked gel can also be used as an endocapsular lens.

### CROSS REFERENCE TO RELATED APPLICATIONS

This application is a continuation-in-part of PCT Application No. PCT/US02/05638 filed Feb. 26, 2002, which was based on, and claimed domestic priority benefits under 35 USC §119(e) from, U.S. Provisional Application Serial No. 60/270,881 filed on Feb. 26, 2001, the entire contents of each prior filed application being expressly incorporated hereinto by reference.

### FIELD OF THE INVENTION

The present invention relates to clinical treatments, such as sealing or repairing wounds, the treatment of other traumatized or degenerative tissue, repair or replacement of organs. In particularly preferred forms, the present invention is specifically embodied in the use of novel crosslinkable polymers, such as dendritic macromolecules and their in vitro, in vivo, and in situ uses. Such uses include ophthalmological, orthopaedic, cardiovascular, pulmonary, skin, or urinary wounds and injuries as well as drug delivery. These biomaterials/polymers are likely to be an effective sealant/glue for other surgical procedures where the site of the wound is not easily accessible or when sutureless surgery is desirable. Crosslinkable dendritic macromolecules can be fabricated into cell scaffold/gel/matrix of specified shapes and sizes using chemical techniques. The polymers, after being crosslinked, can be seeded with cells and then used to repair or replace organs, tissue, or bones. Alternatively, the polymers and cells can be mixed and then injected into the in vivo site and crosslinked in situ for organ, tissue, or bone repair or replacement. The crosslinked polymers provide a three dimensional templates for new cell growth. This method can be used for a variety of reconstructive procedures, including custom molding of cell implants to reconstruct three dimensional tissue defects. The crosslinked gel can also be used as an endocapsular lens. An embodiment of this invention is the preparation of crosslinkable biodendritic macromolecules that can undergo a covalent or non-covalent crosslinking reaction to form a three-deminsional crosslinked gel or network, wherein the crosslinking reaction does not involve a single or multi-photon process. The dendritic polymer can be used for the encapsulation of or the covalent attachment of pharmaceutical agents/drugs including anti-cancer drugs, bioactive peptides, antibacterial compositions, and antinflammatory compounds. The dendritic polymer can be used for drug delivery by itself in a formulation or as part of a crosslinked network.

### BACKGROUND AND SUMMARY OF THE INVENTION

A. Dendritic Macromolecules

Dendritic polymers are globular monodispersed polymers composed of repeated branching units emitting from a central core. (U.S. Pat. Nos. 5,714,166; US 4,289,872; US 4,435,548; US 5,041,516; US 5,362,843; US 5,154,853; US 5,739,256; US 5,602,226; US 5,514,764; Bosman, A. W.; Janssen, H. M.; Meijer, E. W. Chem. Rev. 1999, 99, 1665-1688. Fischer, M.; Vogtle, F. Angew. Chem. Int. Ed. 1999, 38, 884-905. Zeng, F.; Zimmerman, S. C. Chem. Rev. 1997, 97,1681-1712. Tomalia, D. A.; Naylor, A. M.; Goddard, W. A. Angew. Chem. Int. Ed. Engl. 1990, 29,138.) These macromolecules are synthesized using either a divergent (from core to surface) (Buhleier, W.; Wehner, F. V.; Vogtle, F. Synthesis 1987, 155-158. Tomalia, D. A.; Baker, H.; Dewald, J.; Hall, M.; Kallos, G.; Martin, S.; Roeck, J.; Ryder, J.; Smith, P. Polymer Journal 1985, 17, 117-132. Tomalia, D. A.; Baker, H.; Dewald, J.; Hall, M.; Kallos, G.; Martin, S.; Roeck, J.; Ryder, J.; Smith, P. Macromolecules 1986, 19, 2466. Newkome, G. R.; Yao, Z.; Baker, G. R.; Gupta, V. K. J. Org. Chem. 1985, 50, 2003.) or a convergent (from surface to core) (Hawker, C. J.; Frechet, J. M. J. J. Am. Chem. Soc. 1990, 112, 7638-7647) approach This research area has undergone tremendous growth in the last decade since the early work of Tomalia and Newkome. Compared to linear polymers, dendrimers are highly ordered, possess high surface area to volume ratios, and exhibit numerous end groups for functionalization. Consequently, dendrimers display several favorable physical properties for both industrial and biomedical applications including: small polydispersity indexes (PDI), low viscosities, high solubility and miscibility, and excellent adhesive properties. The majority of dendrimers investigated for biomedical/biotechnology applications (e.g., MRI, gene delivery, and cancer treatment) are derivatives of aromatic polyether or aliphatic amides and thus are not ideal for in vivo uses. (Service, R. F. Science 1995, 267, 458-459. Lindhorst, T. K.; Kieburg, C. Angew. Chem. Int. Ed. 1996, 35, 1953-1956. Ashton, P. R.; Boyd, S. E.; Brown, C. L.; Yayaraman, N.; Stoddart, J. F. Angew. Chem. Int. Ed. 1997, 1997, 732-735. Wiener, E. C.; Brechbeil, M. W.; Brothers, H.; Magin, R. L.; Gansow, O. A.; Tomalia, D. A.; Lauterbur, P. C. Magn. Reson. Med. 1994, 31, 1-8. Wiener, E. C.; Auteri, F. P.; Chen, J. W.; Brechbeil, M. W.; Gansow, O. A.; Schneider, D. S.; Beldford, R. L.; Clarkson, R. B.; Lauterbur, P. C. J. Am. Chem. Soc. 1996, 118, 7774-7782. Toth, E.; Pubanz, D.; Vauthey, S.; Helm, L.; Merbach, A. E. Chem. Eur. J. 1996, 2, 1607-1615. Adam, G. A.; Neuerburg, J.; Spuntrup, E.; Muhl;er, A.; Scherer, K.; Gunther, R. W. J. Magn. Reson. Imag. 1994, 4, 462-466. Bourne, M. W.; Margerun, L.; Hylton, N.; Campion, B.; Lai, J. J.; Dereugin, N.; Higgins, C. B. J. Magn. Reson. Imag. 1996, 6, 305-310. Miller, A. D. Angew. Chem. Int. Ed. 1998, 37, 1768-1785. Kukowska-Latallo, J. F.; Bielinska, A. U.; Johnson, J.; Spinder, R.; Tomalia, D. A.; Baker, J. R. Proc. Natl. Acad. Sci. 1996, 93, 4897-4902. Hawthorne, M. F. Angew. Chem. Int Ed. 1993, 32, 950-984. Qualmann, B.; Kessels M. M.; Musiol H.; Sierralta W. D.; Jungblut P. W.; L., M. Angew. Chem. Int. Ed. 1996, 35, 909-911). Biodendrimers are a novel class of dendritic macromolecules composed entirely of building blocks known to be biocompatible or degradable to natural metabolites in vivo. This patent describes the synthesis, characterization, and use of novel dendrimers and dendritic macromolecules called “biodendrimers or biodendritic macromolecules” composed of such biocompatible or natural metabolite monomers such as but not limited to glycerol, Each cited patent and publication cited above and hereinafter is expressly incorporated into the subject application as if set forth fully therein. lactic acid, glycolic acid, succinic acid, ribose, adipic acid, malic acid, glucose, citric acid, etc.

The present invention is generally in the area of the synthesis and fabrication of dendritic polymers and copolymers of polyesters, polyethers, polyether-esters, and polyamino acids or combinations thereof. For example, poly(glycolic acid), poly(lactic acid), and their copolymers are synthetic polyesters that have been approved by the FDA for certain uses, and have been used successfully as sutures, drug delivery carriers, and tissue engineering scaffold for organ failure or tissue loss (Gilding and Reed, Polymer, 20:1459 (1979); Mooney et al., Cell Transpl., 2:203 (1994); and Lewis, D. H. in Biodegradable Polymers as Drug Delivery Systems, Chasin, M., and Langer, R., Eds., Marcel Dekker, New York, 1990). In tissue engineering applications, isolated cells or cell clusters are attached onto or embedded in a synthetic biodegradable polymer scaffold and this polymer-cell scaffold is next implanted into recipients (Langer and Vacanti, Science, 260:920 (1993). A large number of cell types have been used including cartilage cells (Freed et al., Bio/Technology, 12:689 (1994)). Like the novel biodendrimers described in this invention, the advantages include their degradability in the physiological environment to yield naturally occurring metabolic products and the ability to control their rate of degradation by varying the ratio of lactic acid. In the dendritic structures the degradation can be controlled by both the type of monomer used and the generation number.

A further embodiment of this invention is to attach biological recognition units for cell recognition to the end groups or within the dendrimer structure. For example the tripeptide arginine-glycine-aspartic (RGD), can be added to the structure for cell binding. Barrera et al. described the synthesis of a poly(lactic acid) (pLAL) containing a low concentration of N-epsilon.-carbobenzoxy-L-lysine units. The polymers were chemically modified through reaction of the lysine units to introduce arginine-glycine-aspartic acid peptide sequences or other growth factors to improve polymer-cell interactions (Barrera et al., J. Am. Chem. Soc., 115:11010 (1993); U.S. Pat. No. 5,399,665 to Bartera et al.). The greatest limitation in the copolymers developed by Barrera et al. is that only a limited number of lysine units can be incorporated into the backbone. In many tissue engineering applications, the concentration of biologically active molecules attached to the linear polymer is too low to produce the desired interactions between the polymer and the body. Consequently, there is a need for the development of optimal materials for use as scaffolds to support cell growth and tissue development in tissue engineering applications. In addition, there is a need for methods for introducing functionalities such as polyamino acids, peptides, carbohydrates into polyesters, polyether-esters, polycarbonates, etc. in order to improve the biocompatibility and other properties of the polymers. Furthermore there is a need for the development of polyester, polyether ester, polyester-amines, etc materials which include a sufficient concentration of derivatizable groups to permit the chemical modification of the polymer for different biomedical applications.

It is therefore an object of the invention to provide dendritic polymers and copolymers of polyesters and polyamino acids, polyethers, polyurethanes, polycarbonates, polyamino alcohols which can be chemically modified for different biomedical applications such as tissue engineering applications, wound management, contrast agents vehicles, drug delivery vechiles, etc. It is a further object of the invention to provide dendritic polymers and copolymers of polyesters and polyamino acids with improved properties such as biodegradability, biocompatibility, mechanical strength. It is still another object of the invention to provide dendritic polymers that can be derivatized to include functionalities such as peptide sequences or growth factors to improve the interaction of the polymer with cells, tissues, or bone.

The cellular response to conventional linear polymers including adhesion, growth, and/or differentiation of cells cannot be controlled or modified through changes in the polymer's structure, because these polymers (e.g., PLA) do not possess functional groups, other than end groups, that permit chemical modification to change their properties, and these polymers do not adopt a well-defined structure in solution, thereby limiting the applications of these polymers. Consequently the novel polymers described herein are substantially different.

B. Gels

The invention is generally in the area of using dendritic polymeric gels, gel-cell, gel-drug compositions in medical treatments. Gels are 3D polymeric materials which exhibit the ability to swell in water and to retain a fraction of water within the structure without dissolving. The physical properties exhibited by gels such as water content, sensitivity to environmental conditions (e.g., pH, temperature, solvent, stress), soft, adhesivity, and rubbery consistency are favorable for biomedical and biotechnological applications. Indeed, gels may be used as coatings (e.g. biosensors, catheters, and sutures), as “homogeneous” materials (e.g. contact lenses, burn dressings, and dentures), and as devices (e.g. artificial organs and drug delivery systems) (Peppas, N. A. Hydrogel in Medicine and Pharmacy, Vol I and II 1987. Wichterle, O.; Lim, D. Nature 1960, 185, 117-118. Ottenbrite, R. M.; Huang, S. J.; Park, K. Hydrogels and Biodegradable polymers for Bioapplications 1994; Vol. 627, pp 268).

Gel matrices for the entrapment of cells as artificial organs have been explored for more than fifteen years, and microencapsulation is a promising approach for a number of disease states including Parkinson's disease (L-dopamine cells), liver disease (hepatocyte cells), and diabetes (islets of Langerhans). In the past, for example, islets of Langerhans (the insulin producing cells of the pancreas) have embedded encapsulated in an ionically crosslinked alginate (a natural hydrogel) microcapsule with a poly-L-lysine coating, and successfully reduced blood sugar levels in diabetic mice following transplantation.

C. Dendritic Cell Constructs/Scaffolds/Matrices/Gels for Organ/Tissue Repair or Replacement

The present invention is also generally employed in the area of using dendritic polymeric-cell compositions in medical treatments. Several useful examples, which are not to be construed as limiting the present invention, are described below.

Craniofacial contour deformities. Craniofacial contour deformities currently require invasive surgical techniques for correction. These traumatic or congenital deformities are often severe. Alternatively, surgery is requested for an aesthetic personal viewpoint. These deformities often require augmentation in the form of alloplastic prostheses which suffer from problems of infection and extrusion. A minimally invasive method of delivering additional autogenous cartilage or bone to the craniofacial skeleton would minimize surgical trauma and eliminate the need for alloplastic prostheses. By injecting a crosslinkable gel and cells (autoglous or otherwise) one could augment the craniofacial osteo-cartilaginous skeleton with autogenous tissue, without extensive surgery. An embodiment of this inventionis the use of biodendritic cell compositions for treating craniofacial contour deformities.

Breast Tissue Repair of Augmentation. Mammary glands are modified sweat glands attached to the underlying muscle of the anterior chest wall by a layer of connective tissue. A single mammary gland consists of 15-25 lobes, separated by dense connective tissue formed primarily by fibroblasts and bundles of collagen fibers, and adipose tissue containing adipose (fat) cells held together by reticular and collagen fibers. A lactiferous duct that branches extensively is within each lobe. Glandular epithelial cells (alveolar cells) that synthesize and secrete milk into the duct system are located at the ends of the smallest branches. The ducts are composed of simple cuboidal and columnar epithelium. The alveolar cells are embedded in loose connective tissue containing collagen fibers and fibroblasts, lymphocytes, and plasma cells. Close to the alveolar and duct epithelial cells are myoepithelial cells which respond to hormonal and neural stimuli by contracting and expressing the milk. Each lactiferous duct opens onto the surface of the breast through the skin covering the nipple.

Breast surgery can be broadly categorized as either cosmetic or therapeutic. Cosmetic surgeries include augmentation using implants, reduction or reconstruction. Therapeutic surgery is the primary treatment for most early cancers and includes 1) radical surgery that may involve removal of the entire soft tissue anterior chest wall and lymph nodes and vessels extending into the head and neck, 2) lumpectomy, which may involve only a small portion of the breast; and 3) laser surgery for destruction of small regions of tissue. Often reconstructive surgery with implants is used in radical breast surgery. The radical mastectomy involves removal of the breast, both the major and minor pectoralis muscles, and lymph nodes.

Presently, more than 250,000 reconstructive procedures are performed annually, and there are few alternatives to reconstruction as a result of breast cancer, congenital defects, or damage from trauma. Breast reconstruction is frequently used at the time of, or just after, mastectomy for cancer. Reconstructive procedures frequently involve moving vascularized skin flaps with underlying connective and adipose tissue from one region of the body to another. There are numerous surgical methods of breast reconstruction, including tissue expansion followed by silicone implantation, latissimus dorsi flap, pedicled transversus abdominis myocutaneous flap (TRAM), free TRAM flap, and free gluteal flap. Full reconstruction often requires additional procedures over mastectomy and primary reconstruction. These procedures include tissue-expander exchange for permanent implant, revision of reconstruction, nipple reconstruction, and mastopexy/reduction.

Silicone prosthesis that are frequenlty used for reconstruction and augmentation, have afforded many medical complications. It is desirable to have an alternative material for implantation that functions properly, looks and feels like normal tissue, and does not interfere with X-ray diagnosis. It is therefore an object of the invention to provide methods and compositions for reconstruction and augmentation of breast tissue using dendritic polymers or dendritic macromolecules and cell constructs.

Oral tissue repair Oral tissue repair is another area where three-dimensional polymer scaffold/matrices/gels can be used for proliferating oral tissue cells and the formation of components of oral tissues analogous to counterparts found in vivo. These proliferating cells produce proteins, secrete extracellular matrix components, growth factors and regulatory factors necessary to support the long term proliferation of oral tissue cells seeded on the matrix. The production of the fibrous or stromal extracellular matrix tissue that is deposited on the matrix is conducive for the long term growth of the oral tissues in vitro. The three-dimensionality of the scaffold/matrices/gels more closely approximates the conditions in vivo for the particular oral tissues, allowing for the formation of microenvironments encouraging cellular maturation and migration. Specific growth or regulatory factors can also be added to further enhance cell growth and extracellular matrix production.

Tissues of interest include dental pulp, dentin, gingival, submucosa, cementum, periodontal, oral submucosa or tongue tissue cells. The tissue sample subsequently formed is a dental pulp, dentin, gingival submucosa, cementum, periodontal, oral submucosa or tongue tissue sample. The tissue sample may be formed by culturing viable starting cells obtained from an oral tissue sample enriched in dental pulp-derived fibroblasts. In certain aspects of the invention the viable starting cells enriched in dental pulp-derived fibroblasts are obtained from an extracted tooth. Additionally, the tissue sample may be formed by culturing viable starting cells obtained from an oral tissue sample enriched in gingival submucosal fibroblasts, pulp or periodontal ligament fibroblasts as a source of cells. Gingival biopsies are obtainable by routine dental procedures with little or no attendant donor site morbidity. An embodiment of this invention is the use of biodendritic cell compositions for treating oral repair.

It will be understood that the oral tissue sample may again be separated from the matrix prior to application to the patient, or placed in vivo and crosslinked in situ. Equally, the oral tissue sample may be applied in combination with the matrix, wherein the matrix would preferably be a biocompatible matrix. Implantation of a cultured matrix-cell preparation into a specific oral tissue site of an animal to effect reconstruction of oral tissue may involve a biodegradable matrix or a non-biodegradable matrix, depending on the intended function of the preparation.

Urinary incontinence. Urinary incontinence is the most common and the most intractable of all GU maladies. The inability to retain urine and not void urine involuntarily is controlled by the interaction between two sets of muscles. The detrusor muscle, a complex of longitudinal fibers forming the external muscular coating of the bladder, activates the parasympathetic nerves. The second muscle, which is a smooth/striated muscle of the bladder sphincter, and the act of voiding requires the sphincter muscle be voluntarily relaxed at the same time that the detrusor muscle contracts. As one ages, the ability to voluntarily control the sphincter muscle deteriorates. The most common incontinence, particular in the elderly, is urge incontinence where there is only a brief warning before immediate urination. Urge incontinence is a result by a hyperactive detrusor and is typicaly treated with medication and/or “toilet training”. However, reflex incontinence occurs without warning and is usually the result of an impairment of the parasympathetic nerve system. The common incontinence found in elderly women is stress incontinence, which is also observed in pregnant women. This type of incontinence accounts for over half of the total number of cases. Stress incontinence occurs under conditions such as sneezing, laughing or physical effort and is characterized by urine leaking. There are five recognized categories of severity of stress incontinence, designated as types as 0, 1, 2a, 2b, and 3. Type 3 is the most severe and requires a diagnosis of intrinsic sphincter deficiency or ISD (Contemporary Urology, March 1993). There are several treatments including medication, weight loss, exercise, and surgical intervention. The two most common surgical procedures involve either elevating the bladder neck to counteract leakage or constructing a lining from the patient's own body tissue or a prosthetic material such as PTFE to put pressure on the urethra. The second option is to use prosthetic devices such as artificial sphincters to external devices such as intravaginal balloons or penile clamps. The above methods of treatment are very effective for periods typically more than a year. Overflow incontinence is caused by anatomical obstructions in the bladder or underactive detrustors. An embodiment of this invention is the use of biodendritic cell compositions for treating urinary incontinence.

Organ transplantation A cell-scaffold/gel/matrix composition is prepared for in situ polymerization or in vitro use for subsequent implanting to produce functional organ tissue in vivo. The scaffold/gel/matrix is three-dimensional and is composed of crosslinked (covalent, ionic, hydrogen-bondned, etc.) dendritic polymer or copolymer. The scaffold can also be formed from fibers of the dendritic polymer. The cells used are derived from vascularized organ tissue or stem cells and are then suspended in the polymer and subsequently injected in vivo and photocrosslinked to form the gel-cell composite. Alternatively, the cell are attached in vitro to the surface of the preformed crosslinked scaffold or gel to produce functional vascularized organ tissue in vivo. The scaffold/gel/matrix can also be partially chemically degraded with base or acid washings to afford a more hydrophilic material. It is a further embodiment of this invention to separate the linear/dendritic fibers of the woven scaffold by a distance over which diffusion of nutrients and gases can occur typically between 100 and 300 microns. Alternatively, a macroporous gel can be produced by a template, foaming, etc. procedure as described in this invention whereby the uniform or non-uniform pores of 1 to 1000 microns are formed. These gel/scaffold/matrix structures provides for the diffusion and exchange of nutrients, gases, and waste to and from cells proliferating throughout the scaffold in an amount effective to maintain cell viability throughout the material in the absence of vascularization.

Cells attached to the gel/scaffold/matrix may be lymphatic vessel cells, pancreatic islet cells, hepatocytes, bone forming cells, muscle cells, intestinal cells, kidney cells, blood vessel cells, thyroid cells or cells, of the adrenal-hypothalamic pituitary axis. Besides these types of cells, stem cells can be used that subsequently convert to a desired specific cell type.

For example, diabetes mellitus is a disease caused by loss of pancreatic function. Specifically, the insulin producing beta cells of the pancreas are destroyed and thus serum glucose levels rise to high values. As a result, major problems develop in all systems secondary to the vascular changes. Diabetes is estimated to afflict more than 16,000,000 individuals in the United States. Sadly, this number is growing at an alarming rate of about 600,000 new cases diagnosed every year. Presently, diabetes is the third largest cause of death in the U.S., primarily from micro- and macrovascular complications. These complications include limb amputations, ulceration, vascular damage, kidney failure, strokes, and heart attacks which are a result. The daily injection of insulin was once thought to be an effective treatment for diabetes. However, for individuals who have insulin dependent diabetes mellitus (IDDM) and undergo traditional insulin therapy, these horrific complications still persist. In 1992, the Diabetes Control and Complications Trial (DCCT) reported that tightly regulated glucose reduces the risk of these complications. Yet, intensive insulin treatment is not entirely safe due to increased incidences of hypoglycemic episodes. Eastman and Gordon writing on the implications of the DCCT for diabetes treatment stated “the success of intensive treatment as done in the DCCT is both a triumph and a challenge for the health care system: a triumph because we now know that metabolic control matters, and a challenge because the results were achieved by an integrated team of health care researchers with expertise in medicine, education, nutrition, diabetes, self-management skills and human behavior.” These teams are not and probably will not be available in the future for the treatment of the vast majority of patients with diabetes. Consequently, there is a need for novel technologies such as those described in his invention that will provide normal regulation of blood glucose.

The current method of treatment available to diabetic is exogenous administration of insulin, on a regular basis. However, this treatment still results in imperfect control of blood sugar levels. The experimental approach of whole pancreatic tissue transplantation is high risk. However there is not sufficient number of donor pancreases available for diabetics. After transplantation, the serum glucose appears to be controlled in a more physiological manner. This approach is far better then the transplantation of isolated islet cells themselves. An improvement in recent years, has been the encapsulation of the cells to prevent an immune attack by the host. There is evidence of short term function, but the long term results have been less than satisfactory (D. E. R. Sutherland, Diabetologia 20, 161-18 (1981); D. E. R. Sutherland, Diabetologia 20, 435-500 (1981)). Thus whole organ pancreatic transplantation is the preferred treatment. A further embodiment of this invention is to encapsulate/embed islet cells in a biodendritic crosslinkable polymer and subsequent transplantation in the host.

Another useful application of said biodendritic polymers is in the treatment of hepatic failure. Hepatic failure arises as a result of scaring due to a disease, genetic irregularitites, or from injury. Transplantation is the current solution, and without such treatment the outcome is death. It is estimated that 30,000 people die of hepatic failure every year in the United States, with a cost to society of approximately $14 billion annually.

The indications for a liver transplantation include for example acute fulminant hepatic failure, chronic active hepatitis, biliary atresia, idiopathic cirrhosis, primary biliary cirrhosis, sclerosing cholangitis, inborn errors of metabolism, and some types of malignancy. The current method of treatment involves maintaining the patient until a liver becomes available for transplantation. Transplantation of the whole liver is an increasingly successful surgical manipulation. However, the technical complexity of the surgery, the enormous loss of blood, the postoperative conditions, and expense of the operation make this procedure only available in major medical centers. Given the scarcity of the donor organs, the needs of the patient will not be satisfied, Unfortunately, 30,000 patients die each year of end-stage liver disease. Good artificial hepatic support for patients awaiting transplantation is not widely available. Patients suffering from alcohol-induced liver disease represent another large group of patients awaiting treatment. Today patients with end-stage liver disease as a result of alcohol consumption do not have access to transplantation, since there is a scarcity of donor organs and current healthcare compliances. The mortality rates for cirrhosis vary greatly from country to country, ranging from 7.5 per 100,000 in Finland to 57.2 per 100,000 in France. In the U.S., there has been a 70% increase in the number of deaths over the last 25 years. Furthermore, the morbidity for liver cirrhosis is twenty-eight times higher among serious problem drinkers than among nondrinkers.

The liver and pancreas are not the only vital organ systems for which there is inadequate treatment in the form of replacement or restoration of lost function. For example, loss of the majority of the intestine was a fatal condition in the past. Although patients can now survive with intravenous nutrition supplied via the veins, this is an inadequate approach since many complications arise during care. Patients on total parenteral nutrition can develop fatal liver disease or can develop severe blood stream infections. Intestinal transplantation is not a current option since a large number of lymphocytes in the donor intestine are transferred to the recipients. This affords an immunologic reaction “graft vs. host” disease, in which the lymphocytes from the transplanted intestine attack. This eventually leads to death. A further embodiment of this invention is to use biodendritic crosslinkable polymer treating organ loss or repair.

Diseases of the heart and muscle are also a major cause of morbidity and mortality in the world. Cardiac transplantation has been an increasingly successful technique, but, as in the case of liver transplants, requires immunosuppressant drugs and a donor heart. Although organ transplantation is a current remedy for many indications, the scarcity of donor tissue has increased. For example, only a small number of donors are available in the U.S. for the 800-1,000 children/year who need a liver transplantation. Transplantation is often associated with 1) recipients who are very ill and thus the likelihood for success is diminished 2) a complex surgical procedure typically associated with blood loss, 3) the need for a rapid operation since the preservation time is short. The transplantation of only those parenchymal elements necessary to replace lost function has been proposed as an alternative to whole or partial organ transplantation (P. S. Russell, Ann. Surg. 201(3), 255-262 (1985)). This approach has several attractive features, including avoiding major surgery with its attendant blood loss, anesthetic difficulties, and complications. Since only those cells which supply the needed function are replaced, the problems with passenger leukocytes, antigen presenting cells, and other cell types which may promote the rejection process may be reduced or even avoided. Using this approach, the possibility to use cells in an autotransplantation procedure is possible with cells of the recipient's expanded in culture or stem cells that have differentiated to a specific cell type. For example, Demetriou et al reported successful implantation of hepatocytes attached to collagen coated microcarrier beads (A. A. Demetriou, et al., Science 233,1190-1192 (1986)). A further embodiment of this invention is to use biodendritic crosslinkable polymer for organ transplantation.

Skin is another organ that can be damaged by disease or injury. Skin plays a vital role of protecting the body from fluid loss and disease. Skin grafts have been prepared previously from animal skin or the patient's skin, more recently “artificial skin” formed by culturing epidermal cells. In U.S. Pat. No. 4,485,097 Bell discloses a skin-equivalent material composed of a hydrated collagen lattice with platelets and fibroblasts and cells such as keratinocytes. U.S. Pat. No. 4,060,081, to Yannas et al. discloses a multilayer membrane useful as synthetic skin formed from an insoluble non-immunogenic and a non-toxic material such as a synthetic polymer for controlling the moisture flux of the overall membrane. In U.S. Pat. No. 4,458,678, Yannas et al. describe a process for making a skin-equivalent material wherein a fibrous lattice formed from collagen cross-linked with glycosaminoglycan is seeded with epidermal cells. A disadvantage to the first two methods is that the matrix is formed from a permanent” synthetic polymer. In fact, the limitations of this material are discussed in the authors article published in 1980 (Yannas and Burke J. Biomed. Mater. Res., 14, 65-81 (1980)).

Examples of cells that are suitable for use in this invention include but are not limited to hepatocytes and bile duct cells, islet cells of the pancreas, parathyroid cells, thyroid cells, cells of the adrenal-hypothalmic-pituitary axis including hormone-producing gonadal cells, epithelial cells, nerve cells, heart muscle cells, blood vessel cells, lymphatic vessel cells, kidney cells, and intestinal cells, cells forming bone and cartilage, smooth and skeletal muscle.

It is a further object of the invention to provide a method and means for designing, constructing, and utilizing artificial dendritic matrices as temporary scaffolding for cellular growth and implantation. A further embodiment of the invention to provide biodegradable, non-toxic matrices which can be utilized for cell growth, both in vitro, in vivo, and in situ. The cell scaffold/matrix/gel can be formed in vitro or in situ by crosslinking. It is another object of the present invention to provide a method for configuring and constructing biodegradable artificial matrices such that they not only provide a support for cell growth but allow and enhance vascularization and differentiation of the growing cell mass following implantation. It is yet another object of the invention to provide matrices in different configurations so that cell behavior and interaction with other cells, cell substrates, and molecular signals can be studied in vitro.

Polymeric matrix can be used to seed cells and subsequently implanted to form a cartilaginous structure, as described in U.S. Pat. No. 5,041,138 to Vacanti, et al., but this requires surgical implantation of the matrix and shaping of the matrix prior to implantation to form a desired anatomical structure. Hubbell (U.S. Pat. No. 1,995,000478690) describes linear crosslinkable polymers for mixing with cells, followed by in vivo injection and in situ polymerization, however the polymers are nondendritic structures that lack greater optimization of degradation, crosslinking, and chemical and biological derivitazation.

Endocapsular lens replacement: The human eye is a highly evolved and complex sensory organ. It is composed of a cornea, or clear outer tissue which refracts light lays enroute to the pupil, an iris which controls the size of the pupil thus regulating the amount of light entering the eye, and a lens which focuses the incoming light Through the vitreous fluid to the retia. The retina converts the incoming light into a signal that transmitted through the brain stem to the occipital cortex affording a visual image. The light path from the cornea, through the lens and vitreous fluid to the retina is unobstructed. Any obstruction or loss in clarity within these structures causes scattering or absorption of light rays resulting in diminished visual acuity. For example, the cornea can become damaged resulting in oedema, scarring or abrasions, the lens is susceptible to oxidative damage, trauma and infection, and the vitreous can become cloudy due to hemorrhage or inflammation.

As the body ages, the effects of oxidative damage caused by environmental exposure and endogenous free radical production accumulate resulting in a loss of lens flexibility and denatured proteins that slowly coagulate reducing lens transparency. The natural flexibility of the lens is essential for focusing light onto the retina by a process referred to as accommodation. Accommodation allows the eye to automatically adjust the field of vision for objects at different distances. A common condition known as presbyopia results when the cumulative effects of oxidative damage diminish this flexibility reducing near vision acuity. Presbyopia usually begins to occur in adults during their mid-forties; mild forms are treated with glasses or contact lenses.

Lenticular cataract is a lens disorder resulting from the further development of coagulated protein and calcification. There are four common types of cataracts: senile cataracts associated with aging and oxidative stress, traumatic cataracts which develop after a foreign body enters the lens capsule or following intense exposure to ionizing radiation or infrared rays, complicated cataracts which are secondary to diseases such as diabetes mellitus or eye disorders such as detached retinas, glaucoma and retinitis pigmentosa, and toxic cataracts resulting from medicinal or chemical toxicity. Regardless of the cause, the disease results in impaired vision and may lead to blindness.

Treatment of lens disease and the associated loss of vision requires the surgical removal of the lens involving phakoemulsification followed by irrigation and aspiratio. However, without a lens the eye is unable to focus the incoming light on the retina. Consequently, an artificial lens is used to restore vision. Three types of prosthetic lenses are available: cataract glasses, external contact lenses and IOLs. Cataract glasses have thick lenses, are uncomfortably heavy and cause vision artifacts such as central image magnification and side vision distortion. Contact lenses resolve many of the problems associated with glasses, but require frequent cleaning, are difficult to handle (especially for elderly patients with symptoms of arthritis), and are not suited for persons who have restricted tear production. Intaoclar lenses are used in the majority of cases to overcome the aforementioned difficulties associated with cataract glasses and contact lenses. The prior art is replete with a vast A large number of intraocular lenses are described in the prior art such as that found in the following U.S. Pat. Nos. 4,254,509, 4,298,996, 4,842,601, 4,963,148, 4,994,082, 5,047,051.

U.S. Pat. No. 6,361,561 describes an injectable intraocular lens composed of Polysiloxanes. A suitable polysiloxane composition for the preparation of intraocular lenses by a crosslinking reaction, having a refractive index suitable for restoring the refractive power of the natural crystalline lens is described.

More recently, G. M. Wright and T. D. Talcott in U.S. Pat. Nos. 4,537,943; 4,542,542; and 4,608,050 have disclosed injection by needle of a polymer composition into the lens capsule. The polymeric composition comprises a silicone prepolymer, a cross-linker and a platinum-based catalyst. The composition cures in the lens capsule to an optically clear, gel-like material which may accommodate, or focus, through action of the eye lens muscle. However, a problem with the polymeric composition disclosed by the prior art is that a separate heating step is required to permit removal of the needle from the eye to initiate polymerization at the injection site and thus prevent loss of polymer therefrom. Further, the time of initial cross-linking is on the order of several hours, which involves lengthy immobilization of the eye to permit complete curing.

The U.S. Pat. No. 4,919,151 issued to Grubbs, et al discloses a synthetic polymer for endocapsular lens replacement in an eye. The polymer, which is injected into the lens capsule after removal of the lens, comprises an oxygen-stabilized photosensitive prepolymer. An example of such a prepolymer comprises polyether with urethane linkages with one or both ends capped with a functional group containing at least one double bond, such as an acrylate, a methacrylate, or a styrene. The polymerization reaction is initiated with a photoinitiator such as dimethoxyphenylacetophenone and is quenched in the presence of oxygen. Contrary to the prior art polymers, the time of curing is approximately one minute. The viscosity and thickness of the polymer formed may be tailored to achieve a desired index of refraction of between about 1.3 and 1.6.

The U.S. Pat. No. 5,022,413 issued to Spina, Jr. et al discloses a method for treating cataracts by introducing a lenticular tissue dispersing agent into the opacified lens through a small opening in the lens capsule so that the capsule remains substantially intact. The tissue dispersing agent is contained in the lens by a gel-forming substance which functions to block the opening in the lens capsule, preventing its escape. This treatment is preferably carried out in conjunction with laser induced phacofracture.

D. Tissue Sealants

The dendritic macromolecules of the present invention are also usefully employed as a tissue sealant. This biomaterial is likely to be an effective sealant/glue for other surgical procedures (e.g., leaking blebs, nephrotomy closure, bronchopleural fistula repair, peptic ulcer repair, tympanic membrane perforation repair, etc.) where the site of the wound is not easily accessible or when sutureless surgery is desirable.

Cornea perforation treatment: Corneal perforations afflict a fraction of the population and are produced by a variety of medical conditions (e.g., infection, inflammation, xerosis, neurotrophication, and degeneration) and traumas (chemical, thermal, surgical, and penetrating). Unfortunately, corneal perforations often lead to loss of vision and a decrease in an individual's quality of life. Depending on the type and the origin of the perforation, different treatments are currently available from suturing the wound to a cornea graft. However, this is a difficult surgical procedure given the delicate composition of the cornea and the severity of the wound which increase the likelihood for leakage and severe astigmatism after surgery. In certain cases, perforations that cannot be treated by standard suture procedures, tissue adhesives (glues) are used to repair the wound. This type of treatment is becoming very attractive because the method is the simplest, quickest and safest, and corresponds to the requirement of a quick restoration of the integrity of the globe to avoid further complications. Besides an easy and fast application on the wound, the criteria for an adhesive are to 1) bind to the tissue (necrosed or not, very often wet) with an adequate adhesion force, 2) be non-toxic, 3) be biodegradable or resorbable, 4) be sterilizable and 5) not interfere with the healing process. Various alkyl-cyanoacrylates are available for the repair of small perforations. However, these “super glues” present major inconveniences. Their monomers, in particular those with short alkyl chains, can be toxic with formation of formaldehyde. They also polymerize too quickly leading to applications that might be difficult and, once polymerized, the surface of the glue is rough and hard which leads to patient discomfort and a need to wear contact lens. Even though cyanoacrylate is tolerated as a corneal sealant, a number of complications have been reported including cataract formation, corneal infiltration, glaucoma, giant papillary conjunctivitis, and symblepharon formation. Furthermore, in more than 60% of the patients, additional surgical intervention was needed.

Other glues have also been developed. Adhesive hemostats, based on fibrin, are usually constituted of fibrinogen, thrombin and factor XIII. Systems with fibrinogen and photosensitizers activated with light are also being tested. If adhesive hemostats have intrinsic properties which meet the requirements for a tissue adhesive, autologous products (time consuming in an emergency) or severe treatments before clinical use are needed to avoid any contamination to the patient. An ideal sealant for corneal perforations should 1) not impair normal vision, 2) quickly restore the intraocular pressure, IOP, 3) maintain the structural integrity of the eye, 4) promote healing, 5) adhere to moist tissue surfaces, 6) possess solute diffusion properties which are molecular weight dependent and favorable for normal cornea function, 7) possess rheological properties that allow for controlled placement of the polymer on the wound, and 8) polymerize under mild conditions. A further embodiment of this invention is to use biodendritic crosslinkable polymers for sealing corneal perforations.

The use of sutures has limitations and drawbacks. First, suture placement itself inflicts trauma to corneal tissues, especially when multiple passes are needed. Secondly, although suture material has improved, sutures such as 10-0 nylon (which is the suture of choice in the cornea as well as other in vivo area) can act as a nidus for infection and incite corneal inflammation and vascularization. With persistent inflammation and vascularization, the propensity for corneal scarring increases. Thirdly, corneal suturing often yields uneven healing and resultant regular and irregular astigmatism. Postoperatively, sutures are also prone to becoming loose and/or broken and require additional attention for prompt removal. Finally, effective suturing necessitates an acquired technical skill that can vary widely from surgeon to surgeon and can also involve prolonged operative time.

Laser-assisted in situ keratomileusis (LASIK): Laser-assisted in situ keratomileusis is the popular refractive surgical procedure where a thin, hinged corneal flap is created by a microkeratome blade. This flap is then moved aside to allow an excimer laser beam to ablate the corneal stromal tissue with extreme precision for the correction of myopia (near-sightedness) and astigmatism. At the conclusion of the procedure, the flap is then repositioned and allowed to heal. However, with trauma, this flap can become dislocated prior to healing, resulting in flap striae (folds) and severe visual loss. When this complication occurs, treatment involves prompt replacement of the flap and flap suturing. The use of sutures has limitations and drawbacks as discussed above. These novel adhesives could also play a useful role in the treatment of LASIK flap dislocations and striae (folds). These visually debilitating flap complications are seen not uncommonly following the popular procedure LASIK, and are currently treated by flap repositioning and suturing (which require considerable operative time and technical skill). A tissue adhesive could provide a more effective means to secure the flap.

Retinal holes: Techniques commonly used for the treatment of retinal holes such as cryotherapy, diathermy and photocoagulation are unsuccessful in the case of complicated retinal detachment, mainly because of the delay in the application and the weak strength of the chorioretinal adhesion. Cyanoacrylate retinopexy has been used in special cases. It has also been demonstrated that the chorioretinal adhesion is stronger and lasts longer than the earlier techniques. As noted previously with regard to corneal perforation treatment, the extremely rapid polymerization of cyanoacrylate glues (for example, risk of adhesion of the injector to the retina), the difficulty to use them in aqueous conditions and the toxicity are inconveniences and risks associated with this method. The polymerization can be slowed down by adding iophendylate to the monomers but still the reaction occurs in two to three seconds. Risks of retinal tear at the edge of the treated hole can also be observed because of the hardness of cyanoacrylate once polymerized. A further embodiment of this invention is to use biodendritic crosslinkable polymer for sealing retinal holes.

Leaking blebs: Leaking filtering blebs after glaucoma surgery are difficult to manage and can lead to serious, vision-threatening complications. Leaking blebs can result in hypotony and shallowing of the anterior chamber, choroidal effusion, maculopathy, retinal, and choroidal folds, suprachoroidal hemorrhage, corneal decompensation, peripheral anterior synechiae, and cataract formation. A leaking bleb can also lead to the loss of bleb function and to the severe complications of endophthalmaitis. The incidence of bleb leaks increases with the use of antimetabolites. Bleb leaks in eyes treated with 5-fluorouracil or mitomycin C may occur in as many as 20 to 40% of patients. Bleb leaks in eyes treated with antimetabolities may be difficult to heal because of thin avascular tissue and because of abnormal fibrovascular response. If the leak persists despite the use of conservative management, a 9-0 to 10-0 nylon or absorbable suture on a tapered vascular needle can be used to close the conjunctival wound. In a thin-walled or avascular bleb, a suture may not be advisable because it could tear the tissue and cause a larger leak. Fibrin adhesives have been used to close bleb leaks. The adhesive is applied to conjunctival wound simultaneously with thrombin to form a fibrin clot at the application site. The operative field must be dry during the application because fibrin will not adhere to wet tissue. Cyanoacrylate glue may be used to close a conjuctival opening. To apply the glue, the surrounding tissue must be dried and a single drop of the cyanoacrylate is placed. The operative must be careful not to seal the applicator to the tissue or to seal surrounding tissue with glue given its quick reaction. A soft contact lens is then applied over the glue to decrease patient discomfort. However this procedure can actually worsen the problem if the cyanoacrylate tears from the bleb and causes a larger wound. A further embodiment of this invention is to use biodendritic crosslinkable polymers for sealing leaking blebs.

Corneal transplants: In a corneal transplant the surgeon makes approximately 16 sutures around the transplant to secure the new cornea in place. A sutureless procedure would therefore be highly desirable and would offer the following advantages: (1) sutures provide a site for infection, (2) the sutured cornea takes 3 months to heal before the sutures need to be removed, and (3) the strain applied to the new cornea tissue from the sutures can distort the cornea. A further embodiment of this invention is to use biodendritic crosslinkable polymers for sealing a corneal transplant.

Besides ophthalmological applications these crosslinkable polymers have additional surgical uses when the site of the wound is not easily accessible or when sutureless surgery is desired. These photopolymerizable sealants/glues may be of potential use for urinary tract surgery (nephrotomy closure, urethral repair, hypospadia repair), pulmonary surgery (sealing parenchymal & bronchial leaks, bronchopleural fistula repair, persistent air leak repairs), G.I. tract and stomach surgery (parotid cutaneous fistula, tracheo-oesophageal fistula, peptic ulcer repair), joint surgery (cartilage repair, meniscal repair), heart surgery (cardiac ventricular rupture repair), brain surgery (dural defect repairs), ear surgery (ear drum perforation), and post-surgical drainage reduction (mastectomy, axillary dissection). The ease of application, as well as the ability to quickly and precisely seal a wet or dry wound, means that this material may prove to be superior to the previous glues used in many of the above applications

E. Wound Dressings

In the majority of the cases, the treatment used for wound closure is the classical suture technique. However, depending on the type, the origin of the wound as well as the location of the patient, the use of tissue adhesives (e.g., glues, sealants, patches, films and the like is an attractive alternative to the use of sutures. Beside an easy and fast application on the wound, the criteria for an adhesive are to bind to the tissue (necrosed or not, sometimes wet) with an adequate adhesion force, to be non-toxic, biodegradable or resorbable, sterilizable, selectively permeable to gases, impermeable to bacteria and able to control evaporative water loss. Finally, the two main properties of the adhesive are to protect the wound and to enhance the healing process or at least not prevent it. Numerous sealants have been investigated and used for different clinical applications.

Adhesive hemostats, based on fibrin, are the most common products of biological origin. These sealants are usually constituted of fibrinogen, thrombin and factor XIII, as well as fibrinogen/photosensitizers systems. If their intrinsic properties meet the requirements for a tissue adhesive, autologous products (which are time consuming in emergency) or severe treatments before clinical use are needed to avoid any contamination to the patient.

Synthetic materials, mainly polymers and hydrogels in particular have been developed for wound closure. Alkyl-cyanoacrylates are available for the repair of cornea perforations. One investigator has observed no difference in healed skin incisions that were treated by suture or by ethyl-2-cyanoacrylate-“Mediglue” application. However, these “super glues” present major inconveniences. Their monomers, in particular those with short alkyl chains, are or might be toxic and they polymerize too quickly leading to difficulty in treating the wound. Once polymerized, the surface of the glue is rough and hard. This might involve discomfort to the patient and, for example, in case of cornea perforation treatment, a contact lens needs to be worn. Other materials have been commercialized such as “Biobrane II” (composite of polydimethylsiloxane on nylon fabric) and “Opsite” (polyurethane layer with vinyl ether coating on one side). A new polymeric hemostat (poly-N-acetyl glucosamine) has been studied for biomedical applications such as treatment of gastric varices in order to replace cyanoacrylate (vournakis). Adhesives based on modified gelatin are also found to treat skin wounds. Photopolymerizable poly(ethylene glycol) substituted with lactate and acrylate groups are used to seal air leaks in lung surgery.

F. Prevention of Adhesions

Yet another aspect of the invention provides a method for preventing the formation of adhesions between injured tissues by inserting a barrier composed of a biodendritic polymer or combinations of linear and biodendritic polymers between the injured tissues. This polymeric barrier acts as a sheet or coating on the exposed injured tissue to prevent surgical adhesions (Urry et al., Mat. Res. Soc. Symp. Proc., 292, 253-64 (1993). This polymeric barrier will dissolve over a time course that allows for normal healing to occur without formation of adhesions/scars etc. Adhesion formation is a major post-surgical complication. Today, the incidence of clinically significant adhesion is about 5 to 10 percent with some cases cases as high as 100 percent. Among the most common complications of adhesion formation are obstruction, infertility, and pain. Occasionally, adhesion formation requries a second operative procedure to remove adhesion, further complicating the treatment. Given the wide-spread occurrence of post-surgical adhesions, a number of approaches have been explored for preventing adhesions (Stangel et al., “Formation and Prevention of Postoperative Abdominal Adhesions”, The Journal of Reproductive Medicine, Vol. 29, No. 3, March 1984 (pp. 143-156), and dizerega, “The Cause and Prevention of Postsurgical Adhesions”, published by Pregnancy Research Branch, National Institute of Child Health and Human Development, National Institutes of Health, Building 18, Room 101, Bethesda, Md. 20205.)

A number of procedures have been explored for prevention of post-surgical adhesion including 1) Systemic administration of ibuprofen (e.g., see Singer, U.S. Pat. No. 4,346,108), 2) Parenteral administration of antihistamines, corticosteroids, and antibiotics, 3) Intraperitoneal administration of dextran solution and of polyvinylpyrrolidone solution, 4) Systemic administration of oxyphenbutazone, a non-steroidal anti-inflammatory drug that acts by inhibiting prostaglandin production, and 5) Administration of linear synthetic and natural polymers (Hubell 6060582; Fertil. Steril., 49:1066; Steinleitner et al. (1991) “Poloxamer 407 as an Intraperitoneal Barrier Material for the Prevention of Postsurgical Adhesion Formation and Reformation in Rodent Models for Reproductive Surgery,” Obstetrics and Gynecology, 77(1):48 and Leach et al. (1990) “Reduction of postoperative adhesions in the rat uterine horn model with poloxamer 407”, Am. J. Obstet. Gynecol., 162(5):1317. Linsky et al., 1987 “Adhesion reduction in a rabbit uterine horn model using TC-7,” J. Reprod. Med., 32:17, Diamond et al., 1987 “Pathogenesis of adhesions formation/reformation: applications to reproductive surgery,” Microsurgery, 8:103).

For example, formation of post-surgical adhesions involving organs of the peritoneal cavity and the peritoneal wall is undesirable result of abdominal surgery. This occurs frequently and arises from surgical trauma. During the operation, serosanguinous (proteinaceous) exudate is released which tends to collects in the pelvic cavity (Holtz, G., 1984). If the exudate is not absorbed or lysed within a short period it becomes ingrown with fibroblasts, with subsequent collagen deposition occurs leading to adhesions. It is a further embodiment of this invention to administer dendritic macromolecules or combinations of dendritic macromolecules with linear synthetic or natural polymers including peptides for the prevention of adhesions.

G. Drug Delivery

The concept of drug delivery with dendritic macromolecules has been previously explored, (Liu, M. Frechet, M.J. Pharm. Sci. Technol. Today 1999, 2, 393-401) but the composition of the dendrimers explored was not suited for in vivo application and thus restricts their use to study. In fact these polymers such as PAMAM, have shown increased toxicity with increased generation number. The biodendrimers described in this invention offer many opportunities for designing dendrimers that possess building blocks suitable for in vivo use.

The dendritic polymers of the present invention having pendent heteroatom or functional (e.g., amine, carboxylic acid) groups meet the need for controlling physical properties, derivatizing the polymers with drugs, or altering the biodegradability of the polymers. Therefore, the present invention also includes long and short term implantable medical devices containing the polymers of the present invention. A further embodiment of the present invention, the polymers are combined with a biologically or pharmaceutically active compound (drugs, peptides, nucleic acids, etc) sufficient for effective site-specific or systemic drug delivery (Gutowska et al., J. Biomater. Res., 29, 811-21 (1995) and Hoffman, J. Controlled Release, 6, 297-305 (1987)). The biologically or pharmaceutically active compounds may be physically mixed, embedded in, dispersed in, covalently attached, or adhered to the dendritic macromolecule by hydrogen bonds, salt bridges, ect. Furthermore this invention provides a method for site-specific or systemic drug delivery by implanting in the body of a patient in need thereof an implantable drug delivery device containing a therapeutically effective amount of a biological or pharmaceutical active compound in combination with a polymer of the present invention.

Derivatives of biological or pharmaceutical active compounds, including drugs, can also be attached to the dendritic macromolecule by covalent bonds. This provides for the sustained release of the active compound by means of hydrolysis of the covalent bond between the drug and the polymer backbone as well as by the site of the dug in the dendritic structure (e.g., interior vs. exterior). Many of the pendent groups on the dendritic structure are pH sensitive such as carboxylic acid groups which further controls the pH dependent dissolution rate. Such a dendritic macromolecule may also be used for coating gastrointestinal drug release carriers to protect the entrapped biological or pharmaceutical active compounds such as drugs from degrading in the acidic environment of the stomach. The dendritic polymers of the present invention can be prepared having a relatively high concentration of pendant carboxylic acid groups are stable and insoluble (or slightly soluble) in acidic environments but dissolve/degrade rapidly when exposed to more basic environments. A further embodiment of this invention provides a controlled drug delivery system in which a biologically or pharmaceutically active-agent is physically coated with or covalently attached to a polymer of the invention.

A further embodiment of this invention is the delivery of anticancer drugs using the dendrimer. Cancer is a major cause of death in the United States, with more than 500,000 fatalities occuring annually (Katzung, B., “Basic and Clinical Pharmacology”, 7.sup.th Edition, Appleton & Lange, Stamford Conn., 1998, p. 882). Today, one-third of all the patients are cured with using surgery or radiation therapy, which are quite effective when the tumor has not metastasized. Yet in many cases, these treatments are not an effective cancer management.

Cancer chemotherapy can be curative in certain disseminated neoplasms that have undergone either gross or microscopic spread by the time of diagnosis. These include testicular cancer, diffuse large cell lymphoma, Hodgkin's disease and choriocarcinoma as well as childhood tumors such as acute lymphoblastic leukemia. For other forms of disseminated cancer, chemotherapy provides a palliative rather than curative therapy.

For example, colorectal cancer is the third most common cancer diagnosed in men and women in the United States. The American Cancer Society estimates that about 105,500 new cases of colon cancer (49,000 men and 56,500 women) and 42,000 new cases of rectal cancer (23,800 men and 18,200 women) will be diagnosed in 2003. Colorectal cancer is expected to cause about 57,100 deaths (28,300 men and 28,800 women) during 2003. The 5-year relative survival rate is 90% for people whose colorectal cancer is treated in an early stage, before it has spread. But, only 37% of colorectal cancers are found at that early stage. Once the cancer has spread to nearby organs or lymph nodes, the 5-year relative survival rate goes down to 65%. For people whose colorectal cancer has spread to distant parts of the body such as the liver or lungs, the 5-year relative survival rate is 9%.

Colon,

One category of drugs used for cancer therapy is topoisomerase inhibitors. These compounds inhibit the action of topoisomerase enzymes which play a role in the replication, repair, genetic recombination and transcription of DNA. An example of a topoisomerase inhibitor is camptothecin, a natural compound that interferes with the activity of topoisomerase 1, an enzyme involved in DNA replication and RNA transcription. Camptothecin and the camptothecin analogues topotecan and irinotecan are approved for clinical use.

Camptothecin is a plant alkaloid isolated from trees indigenous to China, and analogs thereof such as 9-aminocamptothecin, 9-nitrocamptothecin, 10-hydroxycamptothecin, 10,11-methylenedioxycamptothecin, 9-nitro-10,11-methylenedioxycamptothecin, 9-chloro-10,11-methylenedioxycamptothecin, 9-amino-10,11-methylenedioxycamptothecin, 7-ethyl-10-hydroxycamptothecin (SN-38), topotecan, DX-8951, Lurtotecan (GII147221C), and other analogs (collectively referred to herein as camptothecin drugs) are presently under study worldwide in research laboratories for treatment of colon, breast, and other cancer.

One problem with camptothecin is its water insolubility, which hinders the delivery of the drug. Numerous analogues of camptothecin have been prepared to improve the compound's water solubility. Another problem with camptothecin and its analogues is that the compounds are susceptible in aqueous environments to hydrolysis at the .alpha.-hydroxy lactone ring. The lactone ring opens to the carboxylate form of the drug, a form that exhibits little activity against topoisomerase I.

Various approaches to improving the stability of camptothecin and its analogues have been described. One approach has been to entrap the compounds in liposomes. For example, Burke (U.S. Pat. No. 5,552,156) describes a liposome composition intended to overcome the instability of camptothecin and its analogues by entrapping the compounds in liposomes having a lipid bilayer membrane which allows the compound to penetrate, or intercalate, into the lipid bilayer. With the compound intercalated into the bilayer membrane, it is removed from the aqueous environment in the core of the liposome and thereby protected from hydrolysis. Another report by Subramanian and Muller (Oncology Research, 7(9):461-469 (1995)) describes a liposome formulation of topotecan and report that in liposome-entrapped form, topotecan is stabilized from inactivation by hydrolysis of the lactone ring. However, the biological activity of the liposome-entrapped drug in vitro has only 60% of the activity of the free drug.

In lab tests and in clinical trials, these camptothecin drugs have aroused considerable interest as a result of their ability to halt the growth of a wide range of human tumors. For example, these drugs exhibit unprecedented high levels of antitumor activities against human colon cancer [Giovanella, et al. Science 246: 1046-1048 (Washington, D.C.)(1989)]. Camptothecin drugs have also been shown to be effective against other experimental cancer types such as lung, breast, and malignant melanoma. Moreover, topoisomerase I inhibitors are also known to be useful in the treatment of HIV.

An embodiment of this invention is the delivery of pharmaceutical agents to a site. The drug can be encapsulated within the dendritic polymer or covalently attached, or bound to the dendrimer through a hydrophobic or electrostatic interaction. Drugs of interest but not limited to are anti-cancer, anti-microbial, anti-inflammatory, growth hormones. The dendrimer may be use by itself or incombination with a polymeric, liposome or other composition for delivery or the dendritic polymer may be crosslinkable and used in a formulation by itself or with other crosslinkable polymer(s) or monomer(s).

H. Crosslinked Gels or Networks

To prepare the dendritic crosslinked gel/network of the present invention, dendrimers or dendritic polymers are crosslinked. For example, the dendritic polymers have been chemically modified to have, two or more functional groups that are capable of reacting with nucleophilic groups, such as primary amino (—NH.sub.2) groups or thiol (—SH) groups, on other polymers. Each functional group on a multifunctionally dendritic polymer is capable of covalently binding with another polymer, thereby effecting crosslinking between the polymers and formation of the network.

Examples of covalently crosslinked networks can be formed by reacting an activated ester (such as an N-hydroxysuccinimide) with an amine (such as a terminal primary or secondary amine, lys, etc.) Thiol or cysteine terminated dendritic structure that forms a disulfide crosslinked network with another thiol or cysteine terminated dendritic(s) or linear polymer(s) will also form a gel. Alternatively, a gel is formed during the reaction of an aldehyde functionalized polymer and a amine functionalized polymer. An additional method is to have a malemimide or vinylsulfone functionalized dendritic polymer react with a thiol functionalized dendritic, linear, comb, or other polymer to form the gel. A functionalized succinimidyl glutarate dendritic polymer with an acid terminated dendritic, linear, comb, or other polymer to from the gel. A acrylate functionalized polymer reacts with an amine or thiol functionalized polymer to form the crosslinked gel. A further embodiment of this invention is the use of a chemical peptide ligation reaction to create a crosslinked gel involving a dendritic polymer. In this reaction an aldehyde or aldehyde-acid reacts with a cysteine functionalized polymer to form a gel or crosslinked network.

I. Biologically Active Agents Within the Dendritic Gel/Network

Preferred active agents for use in the compositions of the present invention include growth factors, such as transforming growth factors (TGFs), fibroblast growth factors (FGFs), platelet derived growth factors (PDGFs), epidermal growth factors (EGFs), connective tissue ctivated peptides (CTAPs), osteogenic factors, and biologically active analogs, fragments, and derivatives of such growth factors. Members of the transforming growth factor (TGF) supergene family, which are multifunctional regulatory proteins, are particularly preferred. Members of the TGF supergene family include the beta transforming growth factors (for example, TGF-.beta.1, TGF-.beta.2, TGF-.beta.3); bone morphogenetic proteins (for example, BMP-1, BMP-2, BMP-3, BMP-4, BMP-5, BMP-6, BMP-7, BMP-8, BMP-9); heparin-binding growth factors (for example, fibroblast growth factor (FGF), epidermal growth factor (EGF), platelet-derived growth factor (PDGF), insulin-like growth factor (IGF)), Inhibins (for example, Inhibin A, Inhibin B); growth differentiating factors (for example, GDF-1); and Activins (for example, Activin A, Activin B, Activin AB).

Biodendrimers based on a core unit and branches which is composed of glycerol and lactic acid, glycerol and glycolic acid, glycerol and succinic acid, glycerol and adapic acid, and glycerol, succinic acid, and PEG represent examples of this class of polymers according to the present invention. Thus, one can build a wide range of structures as shown below. After the core is synthesized, polymers such as PEG and PLA can be attached to the core unit or to a brach to make large starburst or dendritic polymers.

### DETAILED DESCRIPTION OF THE PREFERRED EMBODIMENTS

A more complete understanding of the present invention will be obtained from the following Examples which are intended to be exemplary and non-limiting to the present invention.

### EXAMPLE 1

Synthesis of 2-[(cis-1,3-benzylidene glycerol)-2-propionic acid]-cis-1,3-O-Benzylidene glycerol (10.9 g, 60.4 mmol) was dissolved in 1,4-dioxane (250 mL) followed by the addition of NaH (7.0 g, 0.30 mol). The reaction mixture was stirred at rt for one hour before cooling to 0° C. 2-Bromopropionic acid (8.64 mL, 96 mmol) was then added over a 15 minute period of time. The reaction mixture was allowed to return to rt and then stirred at 50° C. for 12 hours before it was cooled to 0° C. and quenched with ethanol followed by the addition of water (250 mL). The solution was adjusted to 4.0 pH using 1 N HCl and extracted with CH₂Cl₂ (200 mL). This procedure was repeated once again after re-adjusting the pH to 4.0. The combined organic phase was dried with Na₂SO₄, gravity filtered, and evaporated. The solid was stirred in ethyl ether (50 mL) for 45 minutes and cooled to −25° C. for 3 hours before collecting 11.7 g of the white

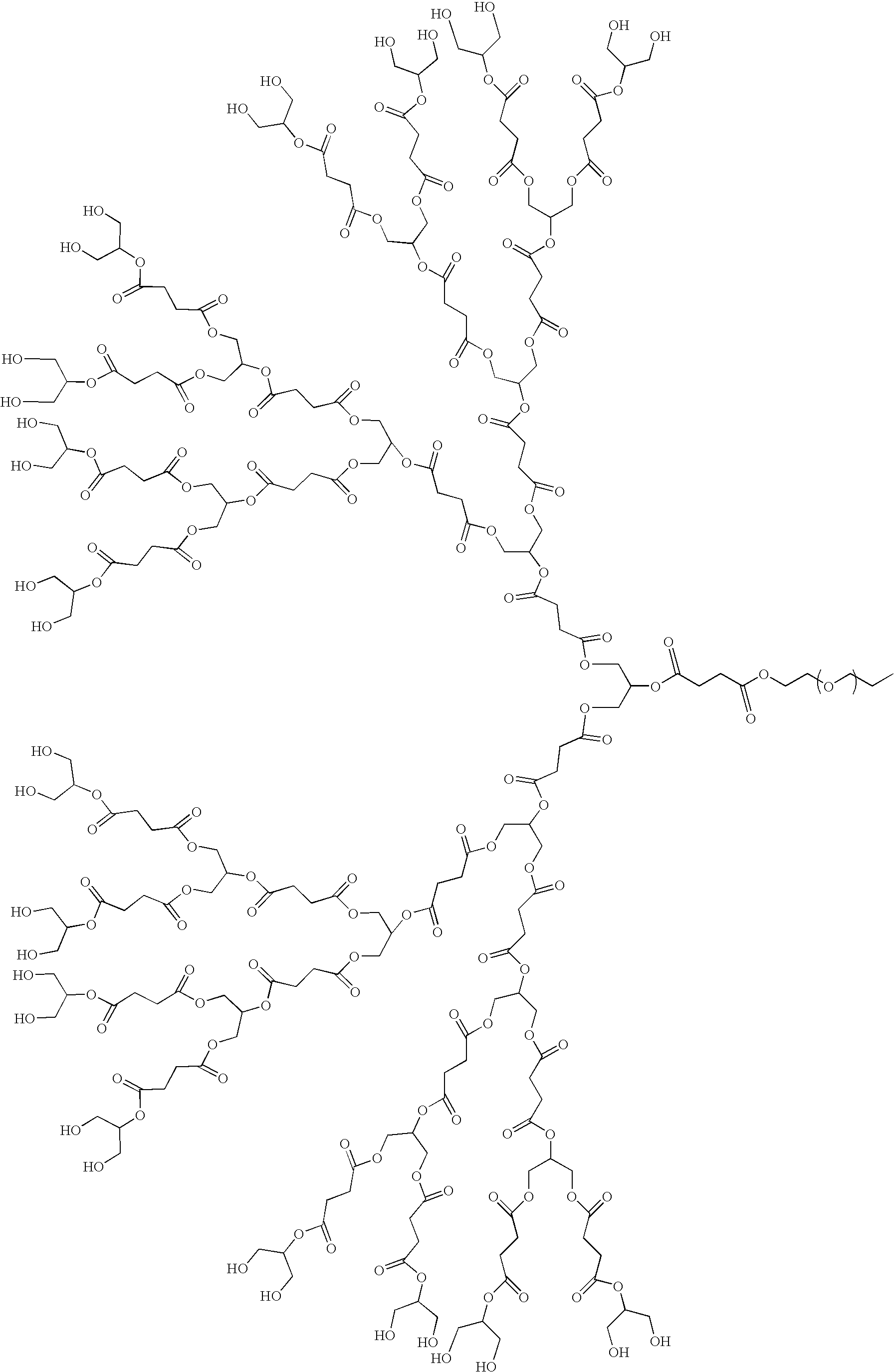

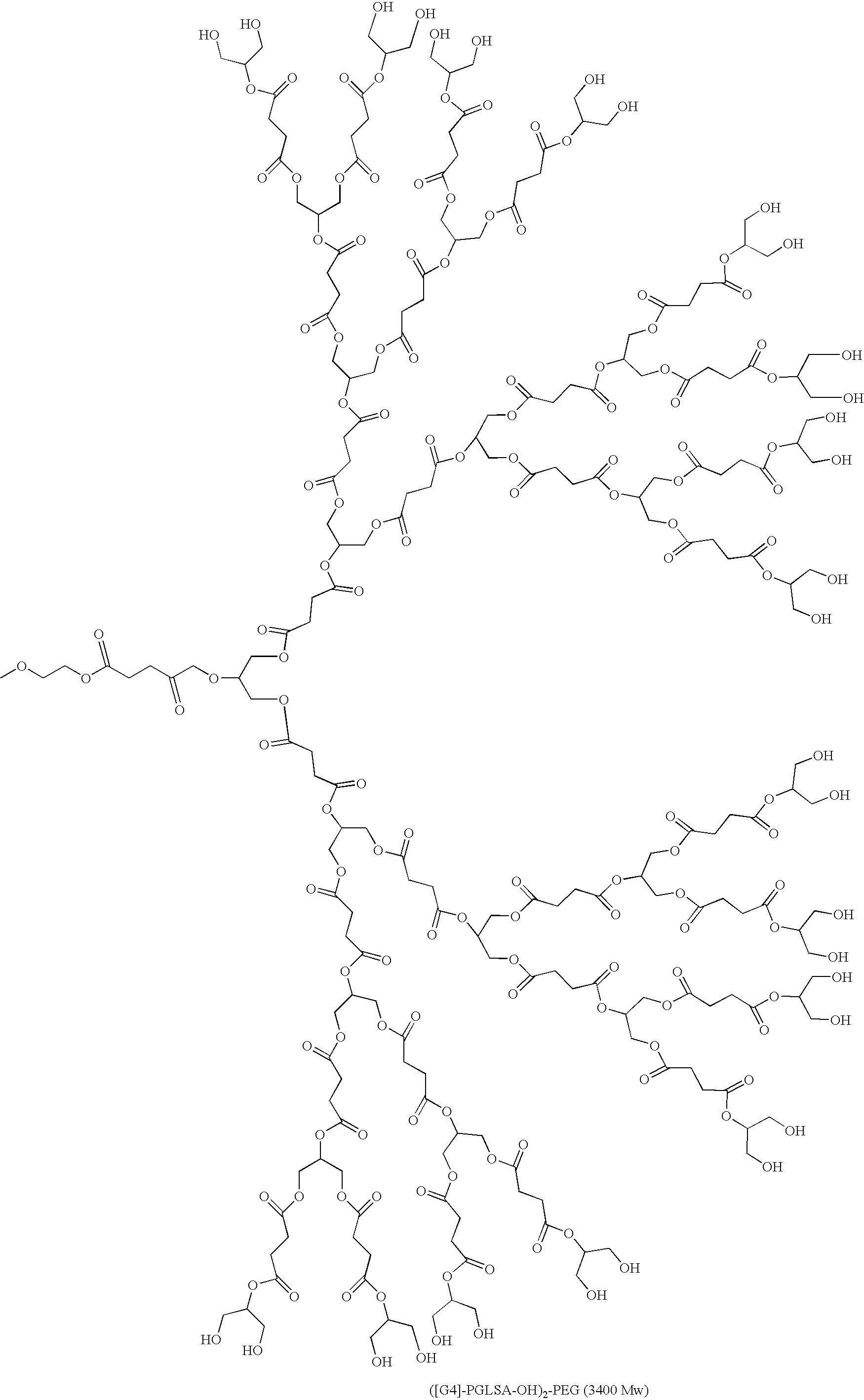

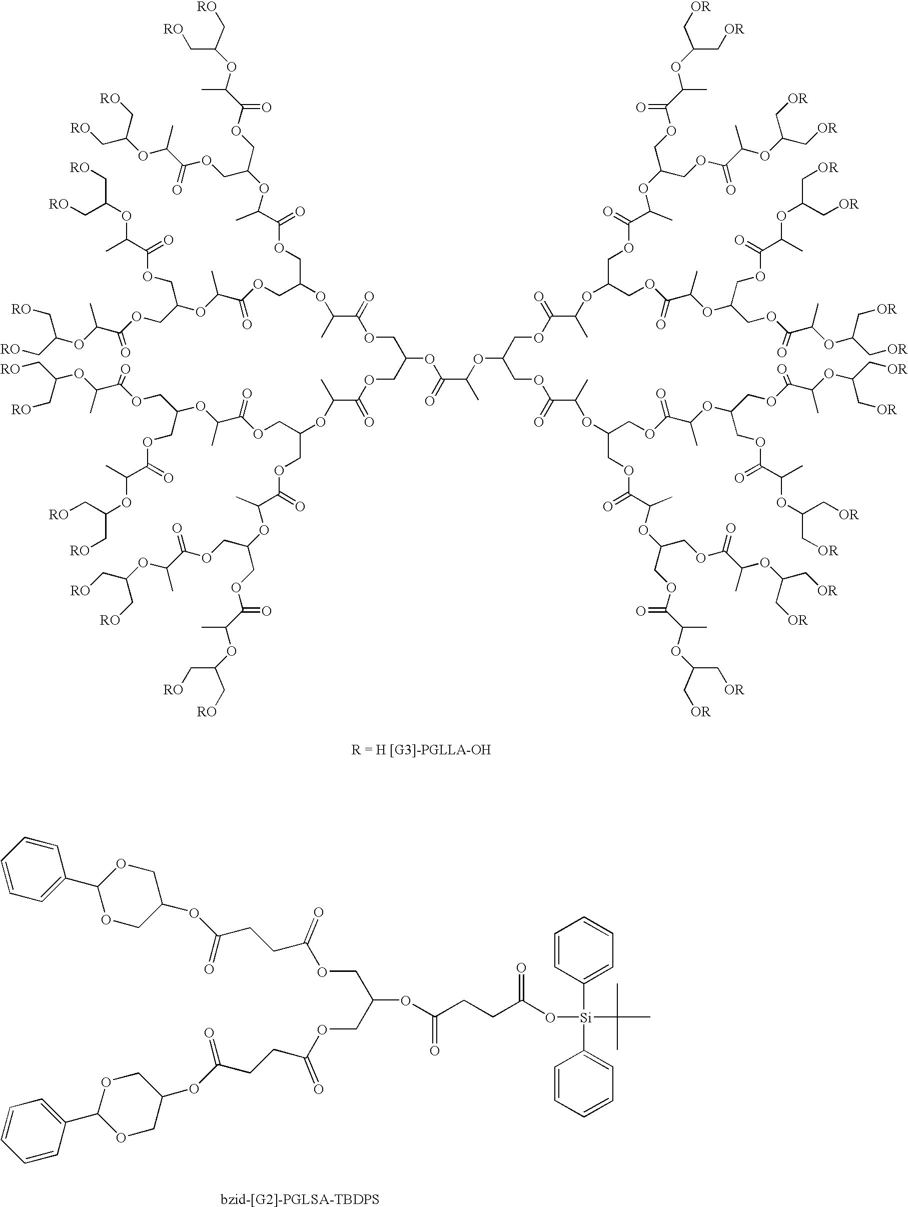

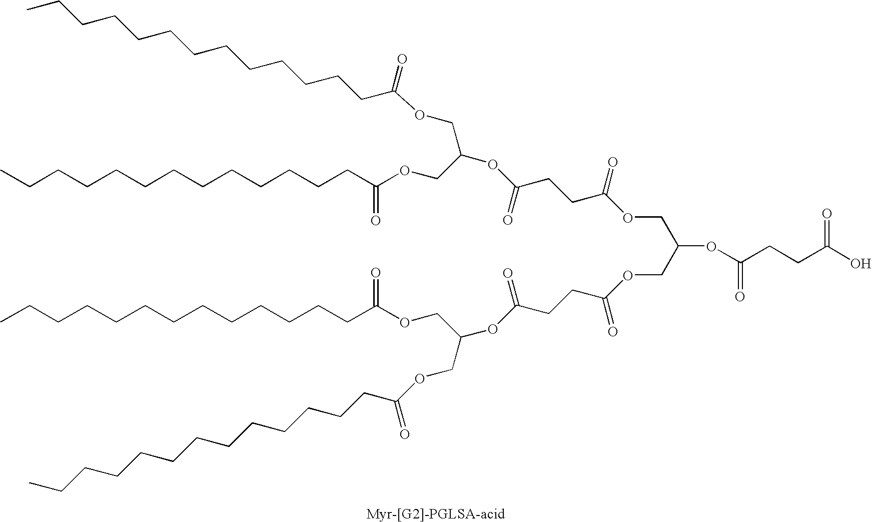

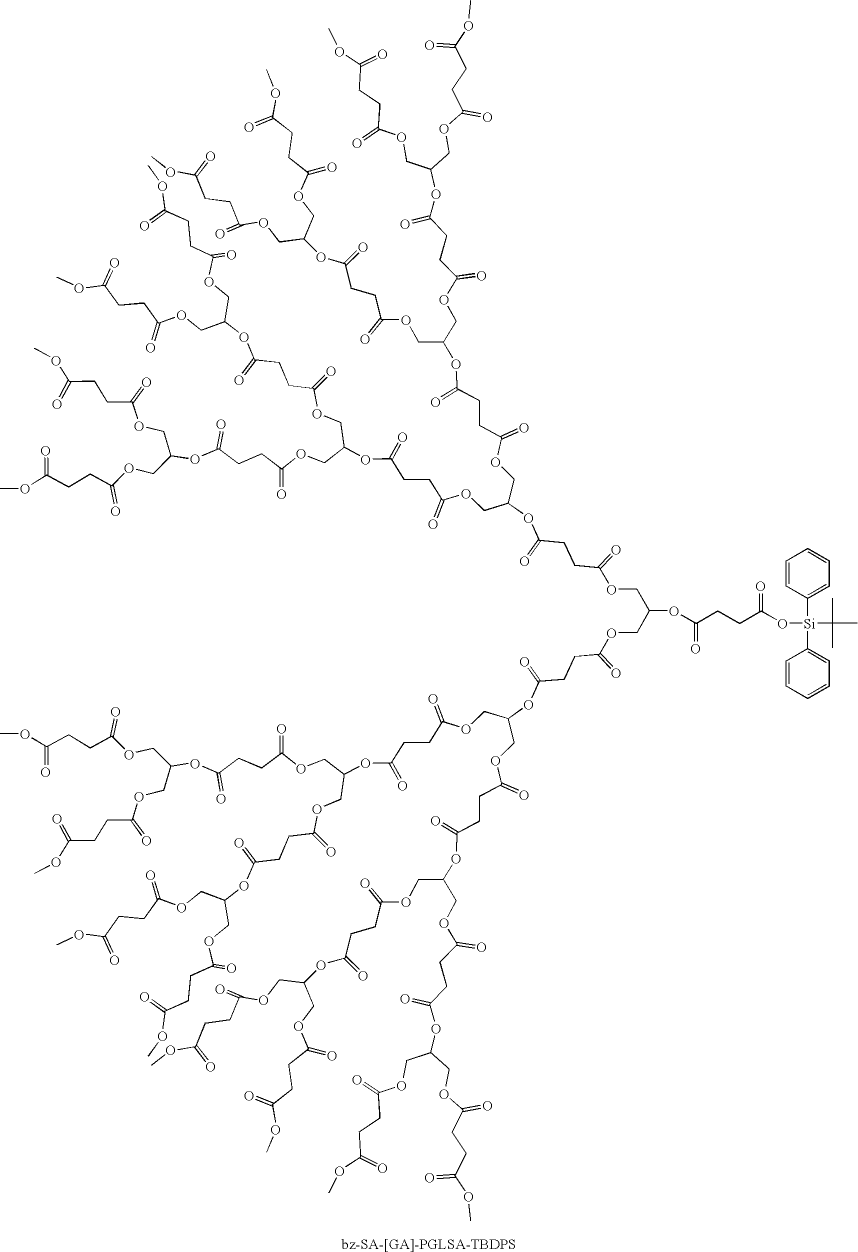

powder (77.3% yield). ¹H NMR obtained GC-MS 253 m/z (MH⁺) (Theory: 252 m/z (M⁺)) Elemental Analysis C: 61.75%; H 6.37% (Theory: C: 61.90%; H 6.39%).

### EXAMPLE 2

Synthesis of benzylidene protected [G0]-PGLLA-bzld—2-[(cis-1,3-benzylidene glycerol)-2-propionic acid] (4.02 g, 15.9 mmol), cis-1,3-β-benzylideneglycerol (2.62 g, 14.5 mmol), and DPTS (1.21 g, 4.10 mmol) were dissolved in CH₂Cl₂ (40 mL). The reaction flask was flushed with nitrogen and then DCC (3.61 g, 17.5 mmol) was added. Stirring at room temperature was continued for 14 hours under a nitrogen atmosphere. Upon reaction completion, the DCC-urea was filtered and washed with a small amount of CH₂Cl₂ (10 mL) and the filtrate was evaporated. The crude product was purified by silica gel chromatography, eluting with 3:97-MeOH:CH₂Cl₂. The product was dissolved in minimal CH₂Cl₂, filtered (to remove any DCU), and precipitated in ethyl ether at −20° C. to remove remaining DCC. Ethyl ether was decanted and the precipitate was exposed to reduced pressure to yield 5.63 g of a white powder (94.0% yield). ¹H NMR obtained GC-MS 415 m/z (MH⁺) (Theory: 414 m/z (M⁺)) Elemental Analysis C: 66.63%; H 6.33% (Theory C: 66.65%; H 6.32%).

### EXAMPLE 3

Synthesis of [G0]-PGLLA-OH—Pd/C (10%) (10% w/w) was added to a solution of benzylidene protected [G0]-PGLLA (5.49 g, 13.2 mmol) in EtOAc/MeOH (3:1, 40 mL). The flask was evacuated and filled with 50 psi of H₂ before shaking for 20 minutes. The catalyst was filtered and washed with EtOAc (10 mL). The filtrate was then evaporated to give 2.94 g of a colorless, viscous oil (94.0% yield). ¹H NMR obtained. (Theory: 238 m/z (M⁺)) Elemental Analysis C: 45.52%; H 7.65% (Theory C: 45.37%; H 7.62%).

### EXAMPLE 4

Synthesis of benzylidene protected [G1]-PGLLA-bzld—2-[(cis-1,3-benzylidene glycerol)-2-propionic acid] (4.41 g, 17.50 mmol), [G0]-PGLLA (0.791 g, 3.32 mmol), and DPTS (2.46 g, 8.36 mmol), were dissolved in DMF (80 mL). The reaction flask was flushed with nitrogen and then DCC (5.31 g, 25.74 mmol) was added. The contents were stirred at room temperature for 14 hours under nitrogen atmosphere. The DMF was removed under high vacuum and the remaining residue was dissolved in CH₂Cl₂. The DCC-urea was filtered and washed with a small amount of CH₂Cl₂ (20 mL) and the filtrate was concentrated. The crude product was purified by silica gel chromatography, eluting with 3:97 MeOH:CH₂Cl₂. The product was dissolved in minimal CH₂Cl₂, filtered (to remove any DCU), and precipitated in ethyl ether at −20° C. to remove remaining DCC. Ethyl ether was decanted and the precipitate was exposed to reduced pressure to yield 3.45 g of a white powder (88.3% yield). ¹H NMR obtained FAB MS 1175.6 m/z (MH⁺) (Theory: 1175.2 m/z (M⁺)) Elemental Analysis C: 62.11%; H 6.46% (Theory C: 62.34%; H 6.35%). SEC Mw: 1280, Mn: 1260, PDI: 1.01.

### EXAMPLE 5

Synthesis of [G1]-PGLLA-OH—Pd/C (10%) (10% w/w) was added to a solution of benzylidene protected [G1]-PGLLA (0.270 g, 0.230 mmol) in THF (15 mL). The flask was evacuated and filled with 50 psi of H₂ before shaking for 15 minutes. The catalyst was filtered and washed with THF (10 mL). The filtrate was then evaporated to give 0.178 g of a colorless, viscous oil (94.0% yield). ¹H NMR obtained FAB MS 823.3 m/z (MH⁺) (Theory: 822.8 m/z (M⁺)) Elemental Analysis C: 47.72%; H 7.41% (Theory C: 48.17%; H 7.11%). SEC M_{w}: 1100, Mₙ: 1090, PDI: 1.01.

### EXAMPLE 6

Synthesis of benzylidene protected [G2]-PGLLA-bzld—2-[(cis-1,3-benzylidene glycerol)-2-propionic acid] (8.029 g, 31.83 mmol), DCC (9.140 g, 44.30 mmol), and DPTS (4.629 g, 15.74 mmol) were dissolved in THF (80 mL). The reaction flask was flushed with nitrogen and stirred for 30 minutes before [G1]-PGLLA (0.825 g, 1.00 mmol) was added by dissolving in a minimal amount of THF. The reaction was stirred at room temperature for 14 hours under nitrogen atmosphere. The DCC-urea was filtered and washed with a small amount of THF (20 mL). The THF filtrate was evaporated and the crude product was purified by silica gel chromatography, eluting with 3:97 MeOH:CH₂Cl₂. The product was dissolved in minimal CH₂Cl₂, filtered (to remove any DCU), and precipitated in ethyl ether at −20° C. to remove remaining DCC. Ethyl ether was decanted and the precipitate was exposed to reduced pressure to yield 2.09 g of a white powder (77% yield). ¹H NMR obtained. FAB MS 2697.0 m/z (MH⁺) (Theory: 2696.8 m/z (M⁺)) Elemental Analysis C: 60.86%; H 6.37% (Theory C: 61.02%; H 6.35%). SEC M_{w}: 2350, Mₙ: 2310, PDI: 1.01.

### EXAMPLE 7

Synthesis of [G2]-PGLLA-OH—Pd/C (10%) (10% w/w) was added to a solution of benzylidene protected [G2]-PGLLA (0.095 g, 0.035 mmol) in THF (10 mL). The flask was evacuated and filled with 50 psi of H₂ before shaking for 15 minutes. The catalyst was filtered and washed with THF (10 mL). The filtrate was evaporated to give 0.061 g of a colorless viscous oil (88.0% yield). ¹H NMR obtained MALDI-TOF MS 1991.8 m/z (MH⁺) (Theory: 1991.9 m/z (M⁺)). SEC M_{w}: 2170, Mₙ: 2130, PDI: 1.01.

### EXAMPLE 8

Synthesis of [G2]-PGLLA-Ac—[G2]-PGLLA (0.098 g, 0.049 mmol) was dissolved in 5 mL of pyridine. Acetic anhydride (6.0 mL, 64 mmol) was then added via syringe and the reaction mixture was stirred at 40° C. for 8 hours. Pyridine and acetic anhydride were removed under high vacuum. The product was isolated on a prep TLC eluting with 4:96 MeOH: CH₃Cl. ¹H NMR obtained. FAB MS 2665.0 m/z (MH⁺) (Theory: 2664.5 m/z (M⁺)) Elemental Analysis C: 50.70%; H 6.71% (Theory C: 50.94%; H 6.43%).

### EXAMPLE 9

Synthesis of benzylidene protected [G3]-PGLLA-bzld—2-[(cis-1,3-benzylidene glycerol)-2-propionic acid] (0.376 g, 1.49 mmol), DCC (0.463 g, 2.24 mmol), and DPTS (0.200 g, 0.680 mmol) were dissolved in THF (15 mL). The reaction flask was flushed with nitrogen and stirred for 1.5 hours before [G2]-PGLLA (0.070 g, 0.035 mmol) was added by dissolving in a minimal amount of THF. The reaction was stirred at room temperature for 14 hours under nitrogen atmosphere. The DCC-urea was filtered and washed with a small amount of THF (20 mL). The THF filtrate was evaporated and the crude product was purified by silica gel chromatography, eluting with 3:97 MeOH:CH₂Cl₂. The product was dissolved in minimal CH₂Cl₂, filtered (to remove any DCU), and precipitated in ethyl ether at −20° C. to remove remaining DCC. Ethyl ether was decanted and the precipitate was exposed to reduced pressure to yield 0.164 g of a white powder (89.1% yield). ¹H NMR obtained MALDI MS 5743.3 m/z (MH⁺) (Theory: 5739.9 m/z (M⁺)) Elemental Analysis C: 60.32%; H 6.34% (Theory C: 60.47%; H 6.36%). SEC M_{w}: 4370, Mₙ: 4310, PDI: 1.01.

### EXAMPLE 10

Synthesis of [G3]-PGLLA-OH—Pd/C (10%) (10% w/w) was added to a solution of benzylidene protected [G3]-PGLLA (0.095 g, 0.035 mmol) in THF (15 mL). The flask was evacuated and filled with 50 psi of H₂ before shaking for 15 minutes. The catalyst was filtered and washed with THF (10 mL). The filtrate was evaporated to give 0.128 g of a colorless viscous oil (95.4% yield). ¹H NMR obtained MALDI MS 4332.5 m/z (MH⁺) (Theory: 4330.2 m/z (M⁺)) Elemental Analysis C: 49.56%; H 7.21% (Theory C: 49.09%; H 6.94%). SEC M_{w}: 4110, Mₙ: 4060, PDI: 1.01.

### EXAMPLE 11

Synthesis of [G0]-PGLSA-bzld—Succinic acid (1.57 g, 13.3 mmol), cis-1,3-O-benzylideneglycerol (5.05 g, 28.0 mmol), and DPTS (4.07 g, 13.8 mmol) were dissolved in CH₂Cl₂ (120 mL). The reaction flask was flushed with nitrogen and then DCC (8.19 g, 39.7 mmol) was added. Stirring at room temperature was continued for 14 hours under a nitrogen atmosphere. Upon reaction completion, the DCC-urea was filtered and washed with a small amount of CH₂Cl₂ (20 mL). The crude product was purified by silica gel chromatography, eluting with 3:97 methanol:CH₂Cl₂. The product was dissolved in CH₂Cl₂, filtered (to remove any DCU), and precipitated in ethyl ether at −20° C. to remove remaining DCC. Following vacuum filtration, 5.28 g of a white solid was collected (90% yield). ¹H NMR (CDCl₃): δ 2.78 (s, 4, —CH₂—CH₂—), 4.08 (m, 4, —CH₂—CH—CH₂—), 4.23 (m, 4, —CH₂—CH—CH₂—), 4.69 (m, 2, —CH₂—CH—CH₂—, J=1.54 Hz, 1.71 Hz), 5.50 (s, 2, CH), 7.34 (m, 6, arom. CH), 7.48 (m, 4, arom. CH). ¹³C NMR (CDCl₃): δ 172.32 (COOR), 138.03 (CH), 129.23 (CH), 128.48 (CH), 126.24 (CH), 101.33 (CH), 69.16 (CH₂), 66.50 (CH), 29.57 (CH₂). FTIR: υ (cm⁻¹) 2992 (aliph. C—H stretch), 1727 (C═O). GC-MS 443 m/z (MH⁺) (Theory: 442 m/z (M⁺)). HR FAB 442.1635 m/z (M⁺) (Theory: 442.1628 m/z (M⁺)). Elemental Analysis C: 65.25%; H 5.85% (Theory C: 65.15%; H 5.92%).

### EXAMPLE 12

Synthesis of [G0]-PGLSA-OH—Pd/C (10% w/w) was added to a solution of benzylidene protected [G0]-PGLSA (2.04 g, 4.61 mmol) in THF (30 mL). The flask for catalytic hydrogenolysis was evacuated and filled with 50 psi of H₂ before shaking for 10 hours. The catalyst was filtered and washed with THF (20 mL). The filtrate was evaporated to give 1.18 g of a clear viscous oil (97% yield). ¹H NMR (CD₃OD): δ 2.67 (s, 4, —CH₂—CH₂—), 3.64 (m, 8, —CH₂—CH—CH₂—), 4.87 (m, 2, —CH₂—CH—CH₂—). ¹³C NMR (CD₃OD): δ 172.77 (COOR), 75.84 (CH₂), 60.41 (CH), 28.96 (CH₂). ¹³C NMR ((CD₃)₂CO): δ 171.99 (COOR), 76.15 (CH₂), 60.89 (CH). FTIR: υ (cm⁻¹) 3299 (OH), 1728 (C═O). GC-MS 284 m/z (M+NH₄⁺) (Theory: 266 m/z (M⁺)). Elemental Analysis C: 44.94%; H 6.87% (Theory C: 45.11%; H 6.81%).

### EXAMPLE 13

Synthesis of 2-(cis-1,3-O-benzylidene glycerol)succinic acid mono ester—cis-1,3-O-Benzylideneglycerol (9.90 g, 54.9 mmol) was dissolved in pyridine (100 mL) followed by the addition of succinic anhydride (8.35 g, 83.4 mmol). The reaction mixture was stirred at room temperature for 18 hours before the pyridine was removed under vacuum at 40° C. The remaining solid was dissolved in CH₂Cl₂ (100 mL) and washed three times with cold 0.2 N HCl (100 mL), or until the aqueous phase remained at pH 1. The organic phase was evaporated and the solid was dissolved in deionized water (300 mL). 1 N NaOH was added until pH 7 was obtained and the product was dissolved in solution. The aqueous phase was extracted with CH₂Cl₂ (200 mL) and then readjusted to pH 4. The aqueous phase was subsequently extracted twice with CH₂Cl₂ (200 mL), dried with Na₂SO₄, filtered, and evaporated. The solid was stirred in ethyl ether (50 mL) and cooled to −25° C. for 3 hours before collecting 14.6 g of a white powder (95% yield). ¹H NMR (CDCl₃): δ 2.68 (m, 4, —CH₂—CH₂—), 4.13 (m, 2, —CH₂—CH—CH₂—), 4.33 (m, 2, —CH₂—CH—CH₂—), 4.70 (m, 1, —CH₂—CH—CH₂—), 5.51 (s, 1, CH), 7.34 (m, 3, arom. CH), 7.47 (m, 2, arom. CH). ¹³C NMR (CDCl₃): δ 178.07 (COOH), 172.38 (COOR), 137.95 (CH), 129.33 (CH), 128.51 (CH), 126.26 (CH), 101.43 (CH), 69.15 (CH₂), 66.57 (CH), 29.24 (CH₂), 29.05 (CH₂). FTIR: υ (cm⁻¹) 2931 (aliph. C—H stretch), 1713 (C═O). GC-MS 281 m/z (MH⁺) (Theory: 280 m/z (M⁺)). Elemental Analysis C: 60.07%; H 5.80% (Theory: C: 59.99%; H 5.75%).

### EXAMPLE 14

Synthesis of [G1]-PGLSA-bzld—2-(cis-1,3-O-Benzylidene glycerol)succinic acid mono ester (6.33 g, 22.6 mmol), [G0]-PGLSA (1.07 g, 4.02 mmol), and DPTS (2.51 g, 8.53 mmol) were dissolved in THF (60 mL). The reaction flask was flushed with nitrogen and then DCC (7.04 g, 34.1 mmol) was added. The reaction was stirred at room temperature for 14 hours under nitrogen atmosphere. Upon completion, the DCC-urea was filtered and washed with a small amount of THF (20 mL) and the solvent was evaporated. The crude product was purified by silica gel chromatography, eluting with 3:97 to 5:95 methanol:CH₂Cl₂. The product was dissolved in CH₂Cl₂, filtered (to remove any DCU), and precipitated in ethyl ether at −20° C. to remove remaining DCC. The ethyl ether was decanted and the precipitate was isolated to yield 5.11 g of a white powder (97% yield). ¹H NMR (CDCl₃): δ 2.58 (m, 4, —CH₂—CH₂—), 2.63 (m, 8, —CH₂—CH₂—), 2.71 (m, 8, —CH₂—CH₂—), 4.12 (m, 12, —CH₂—CH—CH₂—), 4.23 (m, 12, —CH₂—CH—CH₂—), 4.69 (m, 4, —CH₂—CH—CH₂—), 5.20 (m, 2, —CH₂—CH—CH₂—), 5.51 (m, 4, CH), 7.33 (m, 12, arom. CH), 7.46 (m, 8, arom. CH). ¹³C NMR (CDCl₃): δ 172.28 (COOR), 171.91 (COOR), 171.53 (COOR), 138.03 (CH), 129.26 (CH), 128.48 (CH), 126.22 (CH), 101.32 (CH), 69.50 (CH), 69.16 (CH₂), 66.54 (CH), 62.49 (CH₂), 29.36 (CH₂), 29.03 (CH₂). FTIR: υ (cm⁻¹) 2858 (aliph. C—H stretch), 1731 (C═O). FAB MS 1315.6 m/z (MH⁺) (Theory: 1315.3 m/z (M⁺)). Elemental Analysis C: 60.13%; H 5.82% (Theory C: 60.27%; H 5.67%). SEC M_{w}: 1460, Mₙ: 1450, PDI: 1.01.

### EXAMPLE 15

Synthesis of [G1]-PGLSA-OH—Pd/C (10% w/w) was added to a solution of benzylidene protected [G1]-PGLSA (0.270 g, 0.230 mmol) in THF (20 mL). The flask for catalytic hydrogenolysis was evacuated and filled with 50 psi of H₂ before shaking for 10 hours. The catalyst was filtered and washed with THF (20 mL). The filtrate was evaporated to give 0.178 g of a colorless, viscous oil (94% yield). ¹H NMR (CD₃OD):_{—}2.63 (m, 20, —CH₂—CH₂—), 3.52 (m, 4, —CH₂—CH—CH₂—), 3.64 (m, 8, —CH₂—CH—CH₂—), 3.80 (m, 2, —CH₂—CH—CH₂—), 4.05 (m, 2, —CH₂—CH—CH₂—), 4.14 (m, 2, —CH₂—CH—CH₂—), 4.21 (m, 4, —CH₂—CH—CH₂—), 4.30 (m, 4, —CH₂CH—CH₂—), 4.85 (m, 2, —CH₂—CH—CH₂—), 5.25 (m, 2, —CH₂—CH—CH₂—). ¹³C NMR (CD₃OD): δ 172.82 (COOR), 172.58 (COOR), 172.48 (COOR), 172.08 (COOR), 75.82 (CH), 69.90 (CH), 69.68 (CH), 65.66 (CH₂), 62.85 (CH₂), 62.30 (CH₂), 60.43 (CH₂), 28.83 (CH₂), 28.61 (CH₂). FTIR: υ (cm⁻¹) 3405 (OH), 2943 (aliph. C—H stretch), 1726 (C═O). FAB MS 963.2 m/z (MH⁺) (Theory: 962.9 m/z (M⁺)). Elemental Analysis C: 47.13%; H 6.11% (Theory C: 47.40%; H 6.07%). SEC M_{w}: 1510, Mₙ: 1500, PDI: 1.01.

### EXAMPLE 16

Synthesis of [G2]-PGLSA-bzld—2-(cis-1,3-O-Benzylidene glycerol)succinic acid mono ester (4.72 g, 16.84 mmol), [G1]-PGLSA (1.34 g, 1.39 mmol), and DPTS (1.77 g, 6.02 mmol) were dissolved in THF (100 mL). The reaction flask was flushed with nitrogen and then DCC (4.62 g, 22.4 mmol) was added. The reaction was stirred at room temperature for 14 hours under nitrogen atmosphere. Upon completion, the DCC-urea was filtered and washed with a small amount of THF (20 mL) and the solvent was evaporated. The crude product was purified by silica gel chromatography, eluting with 3:97 to 5:95 methanol:CH₂Cl₂. The product was dissolved in CH₂Cl₂, filtered (to remove any DCU), and precipitated in ethyl ether at −20° C. to remove remaining DCC. The ethyl ether was decanted and the precipitate was isolated to yield 4.00 g of a white powder (94% yield). ¹H NMR (CDCl₃): δ 2.59 (broad m, 26, —CH₂—CH₂—), 2.69 (broad m, 52, —CH₂—CH₂—), 4.13 (m, 28, —CH₂—CH—CH₂—), 4.13 (m, 28, —CH₂—CH—CH₂—), 4.69 (m, 8, —CH₂—CH—CH₂—), 5.22 (m, 6, —CH₂—CH—CH₂—), 5.50 (s, 8, CH), 7.32 (m, 24, arom. CH), 7.47 (m, 16, arom. CH). ¹³C NMR (CDCl₃): δ 172.27 (COOR), 171.88 (COOR), 171.60 (COOR), 138.04 (CH), 129.25 (CH), 128.47 (CH), 126.21 (CH), 101.30 (CH), 69.48 (CH), 69.15 (CH₂), 66.54 (CH), 62.57 (CH₂), 29.35 (CH₂), 29.18 (CH₂) 29.03 (CH₂), 28.84 (CH₂). FTIR: υ (cm⁻¹) 2969 (aliph. C—H stretch), 1733 (C—O). FAB MS 3060.7 m/z (MH⁺) (Theory: 3060.9 m/z (M⁺)). Elemental Analysis C: 59.20%; H 5.64% (Theory C: 58.86%; H 5.60%). SEC M_{w}: 3030, Mₙ: 2990, PDI: 1.01.

### EXAMPLE 17

Synthesis of [G2]-PGLSA-OH—Pd/C (10% w/w) was added to a solution of benzylidene protected [G2]-PGLSA (2.04 g, 0.667 mmol) in THF (20 mL). The flask for catalytic hydrogenolysis was evacuated and filled with 50 psi of H₂ before shaking for 10 hours. The catalyst was filtered and washed with THF (20 mL). The filtrate was evaporated to give 1.49 g of a colorless, viscous oil (95% yield). ¹H NMR (CD₃OD): δ 2.64 (m, 52, —CH₂—CH₂—), 3.53 (m, 16, —CH₂—CH—CH₂—), 3.64 (m, 4, —CH₂—CH—CH₂—), 3.80 (m, 8, —CH₂—CH—CH₂—), 4.06 (m, 8, —CH₂—CH—CH₂—), 4.14 (m, 6, —CH₂—CH—CH₂—), 4.21 (m, 11, —CH₂—CH—CH₂—), 4.30 (m, 11, —CH₂—CH—CH₂—), 5.25 (m, 6, —CH₂—CH—CH₂—). ¹³C NMR (CD₃OD): δ 172.83 (COOR), 172.59 (COOR), 172.49 (COOR), 69.91 (CH), 69.69 (CH), 65.68 (CH₂), 62.88 (CH₂), 62.37 (CH₂), 28.61 (CH₂). FTIR: υ (cm⁻¹) 3429 (OH), 2952 (aliph. C—H stretch), 1728 (C═O). MALDI MS 2357.3 m/z (MH⁺) (Theory: 2356.1 m/z (M⁺)). Elemental Analysis C: 48.32%; H 5.97% (Theory C: 47.92%; H 5.90%). SEC M_{w}: 3060, Mₙ: 3000, PDI: 1.02.

### EXAMPLE 18

Synthesis of succinic acid monomethallyl ester (SAME)-2-Methyl-2-propen-1-ol (4.90 mL, 58.2 mmol) was dissolved in pyridine (20 mL) followed by the addition of succinic anhydride (7.15 g, 71.4 mmol). The reaction mixture was stirred at room temperature for 15 hours before the pyridine was removed under vacuum at 30° C. The remaining liquid was dissolved in CH₂Cl₂ (100 mL) and washed two times with cold 0.2 N HCl (100 mL). The organic phase was dried with Na₂SO₄, gravity filtered, and evaporated to give 9.25 g of a clear liquid (92% yield). ¹H NMR (CDCl₃): δ 1.70 (s, 3, CH₃), 2.64 (m, 4, —CH₂—CH₂—), 4.48 (s, 2, —CH₂—), 4.88 (m, 1, vinyl CH₂), 4.93 (m, 1, vinyl CH₂). ¹³C NMR (CDCl₃): δ 178.58 (COOH), 172.05 (COOR), 139.88 (CH), 113.31 (CH₂), 68.31 (CH₂), 29.11 (CH₂), 28.99 (CH₂), 19.59 (CH₃). FTIR: υ (cm⁻¹) 2939 (aliph. C—H stretch), 1711 (C═O). GC-MS 173 m/z (MH⁺) (Theory: 172 m/z (M⁺)). Elemental Analysis C: 55.51%; H 7.09% (Theory: C: 55.81%; H 7.02%).

### EXAMPLE 19

Synthesis of [G2]-PGLSA-SAME—Succinic acid monomethallyl ester (0.826 g, 4.80 mmol), [G2]-PGLSA (0.401 g, 0.170 mmol), and DPTS (0.712 g, 2.42 mmol) were dissolved in THF (50 mL). The reaction flask was flushed with nitrogen and then DCC (1.52 g, 7.37 mmol) was added. Stirring at room temperature was continued for 14 hours under nitrogen atmosphere. Upon completion, the DCC-urea was filtered and washed with a small amount of CH₂Cl₂ (20 mL) and the solvent was evaporated. The crude product was purified by silica gel chromatography, eluting with 3:97 to 5:95 methanol:CH₂Cl₂. The product was dissolved in CH₂Cl₂, filtered (to remove any DCU), and precipitated in ethyl ether at −20° C. to remove remaining DCC. The ethyl ether was decanted and the precipitate was isolated to yield 0.558 g of a clear colorless oil (68.2% yield). ¹H NMR (CDCl₃): δ 1.72 (s, 48, CH₃), 2.63 (m, 116, —CH₂—CH₂—), 4.16 (m, 23, —CH₂—CH—CH₃), 4.27 (m, 23, —CH₂—CH—CH₂—), 4.48 (s, 32, —CH₂—), 4.89 (s, 16, vinyl CH₂), 4.94 (s, 16, vinyl CH₂), 5.24 (m, 14, —CH₂—CH—CH₂—). ¹³C NMR (CDCl₃): δ 171.91 (COOR), 171.67 (COOR), 139.98 (CH), 113.22 (CH₂), 69.43 (CH), 68.31 (CH₂), 62.56 (CH₂), 29.10 (CH₂), 29.02 (CH₂) 28.83 (CH₂), 19.66 (CH₃). FTIR: υ (χm⁻¹) 2969 (aliph. C—H stretch), 1734 (C═O). MALDI MS 4840.9 m/z (MH⁺) (Theory: 4838.7 m/z (M⁺)). Elemental Analysis C: 55.37%; H 6.22% (Theory C: 55.35%; H 6.29%). SEC M_{w}: 5310, Mₙ: 5230, PDI: 1.02.

### EXAMPLE 20

Synthesis of [G3]-PGLSA-bzld—2-(cis-1,3-O-Benzylidene glycerol)succinic acid mono ester (2.77 g, 9.89 mmol), [G2]-PGLSA (1.00 g, 0.425 mmol), and DPTS (1.30 g, 4.42 mmol) were dissolved in THF (40 mL). The reaction flask was flushed with nitrogen and then DCC (2.67 g, 12.9 mmol) was added. The reaction was stirred at room temperature for 14 hours under nitrogen atmosphere. Upon completion, the DCC-urea was filtered and washed with a small amount of THF (20 mL) and the solvent was evaporated. The crude product was purified by silica gel chromatography, eluting with 3:97 to 5:95 methanol:CH₂Cl₂. The product was dissolved in CH₂Cl₂, filtered (to remove any DCU), and precipitated in ethyl ether at −20° C. to remove remaining DCC. The ethyl ether was decanted and the precipitate was isolated to yield 3.51 g of a white powder (90% yield). ¹H NMR (CDCl₃): δ 2.57-2.72 (broad m, 116, —CH₂—CH₂—), 4.12 (m, 60, —CH₂—CH—CH₂—), 4.23 (m, 60, —CH₂CH—CH₂—), 4.68 (m, 16, —CH₂—CH—CH₂—), 5.22 (m, 14, —CH₂—CH—CH₂—), 5.49 (s, 16, CH), 7.33 (m, 48, arom. CH), 7.46 (m, 32, arom. CH). ¹³C NMR (CDCl₃): δ 172.31 (COOR), 171.97 (COOR), 171.65 (COOR), 138.01 (CH), 129.28 (CH), 128.49 (CH), 126.21 (CH), 101.28 (CH), 69.45 (CH), 69.16 (CH₂), 66.53 (CH), 62.59 (CH₂), 29.32 (CH₂), 29.16 (CH₂) 29.01 (CH₂), 28.81 (CH₂). FTIR: υ (cm⁻¹) 2984 (aliph. C—H stretch), 1733 (C═O). MALDI MS 6553.4 m/z (MH⁺) (Theory: 6552.2 m/z (M⁺)). Elemental Analysis C: 58.50%; H 5.66% (Theory C: 58.29%; H 5.57%). SEC M_{w}: 5550, Mₙ: 5480, PDI: 1.01.

### EXAMPLE 21

Synthesis of [G3]-PGLSA-OH—Pd/C (10% w/w) was added to a solution of benzylidene protected [G3]-PGLSA (1.23 g, 0.188 mmol) in 9:1 THF/MeOH (20 mL). The flask for catalytic hydrogenolysis was evacuated and filled with 50 psi of H₂ before shaking for 10 hours. The catalyst was filtered and washed with 9:1 THF/MeOH (20 mL). The filtrate was evaporated to give 0.923 g of a colorless, viscous oil (95% yield). ¹H NMR (CD₃OD): δ 2.64 (m, 116, —CH₂—CH₂—), 3.51 (m, 26, —CH₂—CH—CH₂—), 3.67 (m, 28, —CH₂—CH—CH₂—), 3.80 (m, 12, —CH₂—CH—CH₂—), 4.05 (m, 14, —CH₂—CH—CH₂—), 4.14 (m, 14, —CH₂—CH—CH₂—), 4.22 (m, 22, —CH₂—CH—CH₂—), 4.30 (m, 22, —CH₂—CH—CH₂—), 5.26 (m, 14, —CH₂—CH—CH₂). ¹³C NMR (CD₃OD): δ 172.86 (COOR), 69.91 (CH), 67.64 (CH), 65.67 (CH₂), 62.87 (CH₂), 62.41 (CH₂), 28.61 (CH₂). FTIR: υ (cm⁻¹) 3442 (OH), 2959 (aliph. C—H stretch), 1731 (C═O). MALDI MS 5144.8 m/z (MH⁺) (Theory: 5142.5 m/z (M⁺)). Elemental Analysis C: 48.07%; H 5.84% (Theory C: 48.11%; H 5.84%). SEC M_{w}: 5440, Mₙ: 5370, PDI: 1.01.

### EXAMPLE 22

Synthesis of [G4]-PGLSA-bzld—2-(cis-1,3-O-Benzylidene glycerol)succinic acid mono ester (2.43 g, 8.67 mmol), [G3]-PGLSA (0.787 g, 0.153 mmol), and DPTS (1.30 g, 4.42 mmol) were dissolved in 10:1 THF/DMF (40 mL). The reaction flask was flushed with nitrogen and then DCC (2.63 g, 12.7 mmol) was added. The reaction was stirred at room temperature for 14 hours under nitrogen atmosphere. Upon completion, solvents were removed under vacuum and the remaining solids were redissolved CH₂Cl₂. The DCC-urea was filtered and washed with a small amount of CH₂Cl₂ (20 mL) and the solvent was evaporated. The crude product was purified by silica gel chromatography, eluting with 3:97 to 5:95 methanol:CH₂Cl₂. The product was dissolved in CH₂Cl₂, filtered (to remove any DCU), and precipitated in ethyl ether at −20° C. to remove remaining DCC. The ethyl ether was decanted and the precipitate was exposed to reduced pressure to yield 1.50 g of a white powder (73% yield). ¹H NMR (CDCl₃): δ 2.63 (m, 70, —CH₂—CH₂—), 2.72 (m, 146, —CH₂—CH₂—), 2.90 (m, 32, —CH₂—CH₂—), 4.14 (m, 100, —CH₂—CH—CH₂—), 4.25 (m, 100, —CH₂—CH—CH₂—), 4.70 (m, 32, —CH₂—CH—CH₂—), 5.25 (m, 16, —CH₂—CH—CH₂—), 5.52 (s, 32, CH), 7.33 (m, 96, arom. CH), 7.47 (m, 64, arom. CH). ¹³C NMR (CDCl₃): δ 172.27 (COOR), 171.90 (COOR), 171.57 (COOR), 138.08 (CH), 129.25 (CH), 128.47 (CH), 126.23 (CH), 101.27 (CH), 69.49 (CH), 69.13 (CH₂), 66.54 (CH), 62.45 (CH₂), 29.34 (CH₂), 29.02 (CH₂), 28.83 (CH₂). FTIR: υ (χm⁻¹) 2978 (aliph. C—H stretch), 1733 (C═O). MALDI MS 13536.8 m/z (MH⁺) (Theory: 13534.7 m/z (M⁺)). Elemental Analysis C: 58.20%; H 5.56% (Theory C: 58.04%; H 5.56%). SEC M_{w}: 9000, Mₙ: 8900, PDI: 1.01.

### EXAMPLE 23

Synthesis of [G4]-PGLSA-OH—Pd/C (10% w/w) was added to a solution of benzylidene protected [G4]-PGLSA (0.477 g, 0.0352 mmol) in 9:1 THF/MeOH (20 mL). The flask for catalytic hydrogenolysis was evacuated and filled with 50 psi of H₂ before shaking for 10 hours. The catalyst was filtered and washed with 9:1 THF/MeOH (20 mL). The filtrate was evaporated to give 0.351 g of a colorless, viscous oil (93% yield). ¹H NMR (CD₃OD): δ 2.65 (m, 244, —CH₂—CH₂—), 3.53 (m, 50, —CH₂—CH—CH₂), 3.65 (m, 22, —CH₂—CH—CH₂—), 3.81 (m, 28, —CH₂—CH—CH₂—), 4.05 (m, 32, —CH₂—CH—CH₂—), 4.14 (m, 32, —CH₂—CH—CH₂—), 4.24 (m, 60, —CH₂—CH—CH₂—), 4.30 (m, 60, —CH₂—CH—CH₂—), 5.26 (m, 32, —CH₂—CH—CH₂—). ¹³C NMR (CD₃OD): δ 172.94 (COOR), 69.92 (CH), 65.72 (CH₂), 62.91 (CH₂), 28.67 (CH₂). FTIR: υ (cm⁻¹) 3444 (OH), 2931 (aliph. C—H stretch), 1729 (C═O). MALDI MS 10715.6 m/z (MH⁺) (Theory: 10715.3 m/z (M⁺)). Elemental Analysis C. 48.50%; H 5.83% (Theory C: 48.20%; H 5.81%). SEC M_{w}: 8800, Mₙ: 8720, PDI: 1.01.

### Example 24

### Synthesis of [G0]-PGLAA-bzld

Synthesis of [G0]-PGLAA-bzld—Adipic acid (6.474 g, 44.300 mmol), cis-1,3-O-benzylideneglycerol (17.571 g, 97.508 mmol), and DPTS (10.01 g, 34.03 mmol) were dissolved in DCM (120 mL) followed by the addition of DCC (28.260 g, 136.96 mmol). The reaction was stirred at room temperature for 14 hours under nitrogen atmosphere. Upon reaction completion, the DCC-urea was filtered and washed with a small amount of DCM (50 mL). The crude product was purified by silica gel chromatography, eluting with 2% MeOH in DCM. The appropriate isolated fractions were concentrated, filtered (to remove any DCU), and directly precipitated in hexanes and cooled to −20° C. overnight. Following vacuum filtration, 12.694 g of a white solid was collected (60.8% yield). ¹H NMR (400 MHz, CDCl₃): δ 1.72 (s, 4, —CH₂—CH₂—CH₂—CH₂—), 2.45 (s, 4, —CH₂—CH₂—CH₂—CH₂—), 4.12 (m, 4, —CH₂—CH—CH₁₂—), 4.25 (m, 4, —CH₂—CH—CH₂—), 4.68 (m, 2, —CH₂—CH—CH₂—), 5.52 (s, 2, CH), 7.34 (m, 6, arom. CH), 7.48 (m, 4, arom. CH). ¹³C NMR (100.6 MHz, CDCl₃): δ 173.47 (COOR), 138.01 (CH), 129.27 (CH), 128.50 (CH), 126.22 (CH), 101.43 (CH), 69.30 (CH₂), 66.08 (CH), 34.15 (CH₂), 24.49 (CH₂). FAB 471.2 m/z [M+H]⁺ (Theory: 470.51 m/z [M]⁺).

### EXAMPLE 25

### Synthesis of [G0]-PGLAA-OH

Synthesis of [G0]-PGLAA-OH—Pd(OH)₂/C (10% w/w) was added to a solution of [G0]-PGLAA-bzld (2.161 g, 4.593 mmol) in THF (30 mL). The flask for catalytic hydrogenolysis was evacuated and filled with 60 psi of H₂ before shaking for 10 hours. The catalyst was filtered and washed with THF solution (50 mL). The filtrate was evaporated to give 1.303 g of a clear viscous oil (96.4% yield). ¹H NMR (400 MHz, CD₃OD): δ 1.64 (m, 4, —CH₂—CH₂—CH₂—CH₂—), 2.36 (m, 4, —CH₂—CH₂—CH₂—CH₂—), 3.51 (m, 1, —CH₂—CH—CH₂—), 3.64 (m, 5, —CH₂—CH—CH₂—), 3.78 (m, 1, —CH₂—CH—CH₂—), 4.03 (m, 1, —CH₂—CH—CH₂—), 4.12 (m, 1, —CH₂CH—CH₂—). ¹³C NMR (100.6 MHz, CD₃OD): δ 173.76 (COOR), 75.43 (CH), 69.91 (CH), 65.33 (CH₂), 62.83 (CH₂), 60.49 (CH₂), 33.52 (CH₂), 33.31 (CH₂), 24.12 (CH₂). FAB MS 295.30 m/z [M+H]⁺ (Theory: 294.30 m/z [M]⁺).

### EXAMPLE 26

### Synthesis of Adipic Anhydride

Synthesis of adipic anhydride—Adipic acid (96.28 g, 0.6588 mol) and acetic anhydride (400 mL) were combined and refluxed at 160° C. for four hours. Afterwards, the acetic acid/anhydride was removed under vacuum. Next the depolymerization catalyst, zinc acetate monohydrate, was added along with a distillation apparatus and the heat was slowly increased. After 100° C., nothing was collected until 200° C. when 68.79 g of a clear colorless liquid was collected (82.5% yield). ¹H NMR (400 MHz, CDCl₃): δ 1.91 (m, 4, —CH₂—CH₂—CH₂—CH₂—), 2.67 (m, 4, —CH₂—CH₂—CH₂—CH₂—). ¹³C NMR (100.6 MHz, CDCl₃): δ 168.38 (—COOCO—), 34.60 (CH₂), 22.37 (CH₂). GC-MS 128 m/z [M]⁺ (Theory: 128.12 m/z [M]⁺).

### EXAMPLE 27

### Synthesis of 2-(cis-1,3-O-benzylidene glycerol)adipic Acid Mono Ester

cis-1,3-O-benzylideneglycerol (68.74 g, 0.5365 mol) was dissolved in pyridine (150 mL) followed by the addition of adipic anhydride (82.50 g, 0.4578 mol). The reaction mixture was stirred at room temperature for 18 hours before the pyridine was removed under vacuum at 35° C. The remaining solid was dissolved in DCM (400 mL) and washed two times with 0.2 N HCl (400 mL), or until the aqueous phase remained at pH 1. The organic phase was evaporated and the solid was added to deionized water (300 mL). 1 N NaOH was added until pH 7 was obtained and the product was in the aqueous solution. The aqueous phase was washed with DCM (400 mL), to extract any remaining adipic anhydride, and then readjusted to pH 4. The aqueous phase was subsequently extracted twice with DCM (400 mL), dried with Na₂SO₄, filtered, and evaporated to afford 67.53 g of a white powder (47.80% yield). ¹H NMR (400 MHz, CDCl₃): δ 1.70 (m, 4, —CH₂—CH₂—CH₂—CH₂—), 2.35 (m, 2, —CH₂—CH₂—CH₂—CH₂—), 2.44 (m, 2, —CH₂—CH₂—CH₂—CH₂—), 4.13 (m, 2, —CH₂—CH—CH₂—), 4.25 (m, 2, —CH₂—CH—CH₂—), 4.67 (m, 1, —CH—CH—CH₂—), 5.53 (s, 1, CH), 7.33 (m, 3, arom. CH), 7.47 (m, 2, arom. CH). ¹³C NMR (100.6 MHz, CDCl₃): δ 178.98 (COOH), 173.48 (COOR), 137.97 (CH), 129.30 (CH), 128.51 (CH), 126.22 (CH), 101.45 (CH), 69.28 (CH₂), 66.13 (CH), 34.13 (CH₂), 33.71 (CH₂), 24.43 (CH₂), 24.21 (CH₂). FAB MS 309.1 m/z (MH⁺) (Theory: 308.33 m/z (M⁺)).

### EXAMPLE 28

### Synthesis [G1]-PGLAA-bzld

First, 2-(cis-1,3-O-benzylidene glycerol)adipic acid mono ester (7.226 g, 23.434 mmol), [G0]-PGLAA-OH (1.222 g, 4.152 mmol), and DPTS (2.830 g, 9.621 mmol) were dissolved in THF (100 mL) followed by the addition of DCC (4.32 g, 21.0 mmol). The reaction was stirred at room temperature for 14 hours under nitrogen atmosphere. Upon reaction completion, the DCC-urea was filtered and washed with a small amount of THF (50 mL). The crude product was purified by silica gel chromatography, eluting with 1/1 to 4/1 EtOAc:hexanes. The appropriate isolated fractions were concentrated, filtered (to remove any DCU), and directly precipitated in hexanes and cooled to −20° C. overnight. The hexanes were decanted and the precipitate was isolated to yield 5.99 g of a sticky solid (99.1% yield). ¹H NMR (400 MHz, CDCl₃): δ 1.63 (m, 20, —CH₂—CH₂—CH₂—CH₂—), 2.32 (m, 12, —CH₂—CH₂—CH₂—CH₁₂—), 2.43 (m, 8, —CH₂—CH₂—CH₂—CH₂—), 4.10 (m, 12, —CH₂—CH—CH₂—), 4.25 (m, 12, —CH₂—CH—CH₂—), 4.68 (m, 4, —CH₂—CH—CH₂—), 5.21 (m, 2, —CH₂—CH—CH₂—), 5.51 (s, 4, CH), 7.32 (m, 12, arom. CH), 7.47 (m, 8, arom. CH). ¹³C NMR (100.6 MHz, CDCl₃): δ 173.40 (COOR), 172.87 (COOR), 172.55 (COOR), 138.02 (CH), 129.28 (CH), 128.49 (CH), 126.21 (CH), 101.39 (CH), 69.28 (CH₂), 66.11 (CH), 62.39 (CH₂), 34.08 (CH₂), 33.90 (CH₂), 33.75 (CH₂), 24.37 (CH₂). FAB MS 1455.6 m/z [M+H]⁺ (Theory: 1455.54 m/z [M]⁺).

### EXAMPLE 29

### Synthesis [G1]-PGLAA-OH

Synthesis of [GI]-PGLAA-OH—Pd(OH)₂/C (10% w/w) was added to a solution of [G1]-PGLAA-bzld (4.870 g, 3.346 mmol) in THF (50 mL). The flask for catalytic hydrogenolysis was evacuated and filled with 60 psi of H₂ before shaking for 10 hours. The catalyst was filtered and washed with THF solution (50 mL). The filtrate was evaporated to give 3.669 g of a clear viscous oil (99.5% yield). ¹H NMR (400 MHz, CD₃OD): δ 1.63 (m, 20, —CH₂—CH₂—CH₂—CH₂—), 2.36 (m, 20, —CH₂—CH₂—CH₂—CH₂—), 3.52 (m, 2, —CH₂—CH—CH₁₂—), 3.59-3.69 (broad m, 12, —CH₂—CH—CH₂—), 3.79 (m, 1, —CH₂—CH—CH₂—), 4.03 (m, 1, —CH₂—CH—CH₁₂—), 4.14 (m, 5, —CH₂—CH—CH₂—), 4.32 (m, 4, —CH₂—CH—CH₂—), 5.24 (m, 2, —CH₂—CH—CH₂—). ¹³C NMR (100.6 MHz, CD₃OD): δ 173.64 (COOR), 173.36 (COOR), 172.93 (COOR), 75.42 (CH), 69.93 (CH), 69.47 (CH), 65.36 (CH₂), 62.87 (CH₂), 62.15 (CH₂), 60.50 (CH₂), 33.49 (CH₂), 33.35 (CH₂), 33.20 (CH₂), 24.11 (CH₂). MALDI-TOF MS 1125.8 m/z [M+Na]⁺ (Theory: 1103.11 m/z [M]⁺).

### EXAMPLE 30

### Synthesis [G2]-PGLAA-bzld

Synthesis of [G2]-PGLAA-bzld—2-(cis-1,3-O-benzylidene glycerol)adipic acid mono ester (10.012 g, 32.472 mmol), [G1]-PGLAA-OH (3.397 g, 3.079 mmol), and DPTS (2.508 g, 8.527 mmol) were dissolved in THF (100 mL) followed by the addition of DCC (4.62 g, 22.4 mmol). The reaction was stirred at room temperature for 14 hours under nitrogen atmosphere. Upon reaction completion, the DCC-urea was filtered and washed with a small amount of THF (50 mL). The crude product was purified by silica gel chromatography, eluting with 2% MeOH in DCM. The appropriate isolated fractions were concentrated, filtered (to remove any DCU), and directly precipitated in hexanes and cooled to −20° C. overnight. The hexanes were decanted and the precipitate was isolated to yield 9.39 g of a sticky wax (89.0% yield). ¹H NMR (400 MHz, CDCl₃): δ 1.63 (m, 52, —CH₂—CH₂—CH₂—CH₂—), 2.31 (m, 36, —CH₂—CH₂—CH₂—CH₂—), 2.41 (m, 16, —CH₂—CH₂—CH₂—CH₂—), 4.05 (m, 28, —CH₂—CH—CH₂—), 4.25 (m, 28, —CH₂—CH—CH₂—), 4.67 (m, 8, —CH₂—CH—CH₂—), 5.21 (m, 6, —CH₂—CH—CH₂—), 5.51 (s, 8, CH), 7.33 (m, 24, arom. CH), 7.46 (m, 16, arom. CH). ¹³C NMR (100.6 MHz, CDCl₃): δ 173.39 (COOR), 172.87 (COOR), 172.54 (COOR), 138.02 (CH), 129.27 (CH), 128.49 (CH), 126.21 (CH), 101.38 (CH), 69.27 (CH₂), 66.11 (CH₂), 62.39 (CH₂), 34.08 (CH₂), 33.74 (CH₂), 33.67 (CH₂), 24.37 (CH₂). MALDI MS 3449.2 m/z [M+Na]⁺ (Theory: 3425.61 m/z [M]⁺).

### EXAMPLE 31

### Synthesis [G2]-PGLAA-OH

Synthesis of [G2]-PGLAA-OH—Pd(OH)₂/C (10% w/w) was added to a solution of [G2]-PGLAA-bzld (8.02 g, 2.34 mmol) in THF (100 mL). The flask for catalytic hydrogenolysis was evacuated and filled with 60 psi of H₂ before shaking for 10 hours. The catalyst was filtered and washed with THF solution (50 mL). The filtrate was evaporated to give 6.360 g of a clear viscous oil (99.4% yield). ¹H NMR (400 MHz, CD₃OD): δ 1.62 (m, 52, —CH₂—CH₂—CH₂—CH₂—), 2.35 (m, 52, —CH₂—CH₂—CH₂—CH₂—), 3.52 (m, 5, —CH₂—CH—CH₂—), 3.59-3.71 (broad m, 25, —CH₂—CH—CH₂—), 3.79 (m, 3, —CH₂—CH—CH₂—), 4.03 (m, 3, —CH₂—CH—CH₂—), 4.14 (m, 15, —CH₂—CH—CH₂—), 4.33 (m, 12, —CH₂—CH—CH₂—), 5.25 (m, 6, —CH, —CH—CH₂—). ¹³C NMR (100.6 MHz, CD₃OD): δ 173.63 (COOR), 173.27 (COOR), 172.92 (COOR), 75.42 (CH), 69.94 (CH), 69.47 (CH), 65.38 (CH₂), 62.89 (CH₂), 62.17 (CH₂), 60.52 (CH₂), 33.51 (CH₂), 33.39 (CH₂), 33.22 (CH₂), 24.12 (CH₂). MALDI-TOF MS 2744.3 m/z [M+Na]⁺ (Theory: 2720.75 m/z [M]⁺).

### EXAMPLE 32

### Synthesis [G3]-PGLAA-bzld

Synthesis of [G3]-PGLAA-bzld—2-(cis-1,3-O-benzylidene glycerol)adipic acid mono ester (12.626 g, 40.950 mmol), [G2]-PGLAA-OH (5.263 g, 1.934 mmol), and DPTS (3.232 g, 10.989 mmol) were dissolved in THF (100 mL) followed by the addition of DCC (12.581 g, 60.975 mmol). The reaction was stirred at room temperature for 14 hours under nitrogen atmosphere. Upon reaction completion, the DCC-urea was filtered and washed with a small amount of THF (60 mL). The crude product was purified by silica gel chromatography, eluting with 1.5 to 3.0% MeOH in DCM. The appropriate isolated fractions were concentrated, filtered (to remove any DCU), and directly precipitated in hexanes and cooled to −20° C. overnight. The hexanes were decanted and the precipitate was isolated to yield 12.22 g of a sticky wax (85.8% yield). ¹H NMR (400 MHz, CDCl₃): δ 1.63 (broad m, 130, —CH₂—CH₂—CH₂—CH₂—), 2.31 (m, 90, —CH₂—CH₂—CH₂—CH₂—), 2.41 (m, 32, —CH₂—CH₂—CH₂—CH₂—), 4.10 (m, 62, —CH₂—CH—CH₂—), 4.24 (m, 62, —CH₂—CH—CH₂—), 4.67 (m, 16, —CH₂—CH—CH₂—), 5.19 (m, 14, —CH—CH—CH₂—), 5.51 (s, 16, CH), 7.32 (m, 48, arom. CH), 7.46 (m, 32, arom. CH). ¹³C NMR (100.6 MHz, CDCl₃): δ 173.38 (COOR), 172.89 (COOR), 172.48 (COOR), 138.03 (CH), 129.27 (CH), 128.49 (CH), 126.21 (CH), 101.36 (CH), 69.26 (CH₂), 66.11 (CH), 62.29 (CH₂), 34.08 (CH₂), 33.83 (CH₂), 33.74 (CH₂), 33.67 (CH₂), 24.43 (CH₂), 24.36 (CH₂). MALDI-TOF MS 7390 m/z [M+Na]⁺ (Theory: 7365.73 m/z [M]⁺).

### EXAMPLE 33

### Synthesis [G3]-PGLAA-OH

Synthesis of [G3]-PGLAA-OH—Pd(OH)₂/C (10% w/w) was added to a solution of [G3]-PGLAA-bzld (11.03 g, 1.497 mmol) in THF (125 mL). The flask for catalytic hydrogenolysis was evacuated and filled with 60 psi of H₂ before shaking for 10 hours. The catalyst was filtered and washed with THF solution (75 mL). The filtrate was evaporated to give 8.69 g of a clear viscous oil (97.5% yield). ¹H NMR (400 MHz, CD₃OD): δ 1.63 (m, 124, —CH₂—CH₂—CH₂—CH₂—), 2.35 (m, 127, —CH₂—CH₂—CH₂—CH₂—), 3.52 (m, 7, —CH₂—CH—CH₂—), 3.60-3.71 (broad m, 55, —CH₂—CH—CH₂—), 3.79 (m, 4, —CH₂—CH—CH₂—), 4.04 (m, 5, —CH₂—CH—CH₂—), 4.14 (m, 34, —CH₂—CH—CH₂—) 4.32 (m, 29, —CH₂—CH—CH₂—), 5.25 (m, 14, —CH, —CH—CH₂—). ¹³C NMR (100.6 MHz, CD₃OD): δ 173.82 (COOR), 173.63 (COOR), 173.36 (COOR), 173.27 (COOR), 172.92 (COOR), 75.45 (CH), 75.40 (CH), 69.96 (CH), 69.48 (CH), 65.40 (CH₂), 62.92 (CH₂), 62.23 (CH₂), 60.54 (CH₂), 33.53 (CH₂), 33.25 (CH₂), 24.15 (CH₂). MALDI-TOF MS 5975.0 m/z [M+Na]⁺ (Theory: 5956.02 m/z [M]⁺).

### EXAMPLE 34

### Synthesis of [G0]-PGLSA-[G1]-PGLAA-bzld

Synthesis of [G0]-PGLSA-[G1]-PGLAA-bzld—2-(cis-1,3-O-benzylidene glycerol)adipic acid mono ester (11.793 g, 38.248 mmol), [G0]-PGLSA-OH (1.185 g, 4.449 mmol), and DPTS (2.853 g, 9.700 mmol) were dissolved in THF (50 mL) followed by the addition of DCC (7.216 g, 34.973 mmol). The reaction was stirred at room temperature for 14 hours under nitrogen atmosphere. Upon completion, the DCC-urea was filtered and washed with a small amount of THF (50 mL) and the solvent was evaporated. The crude product was purified by silica gel chromatography, eluting with 1/1 to 4/1 EtOAc:hexanes. The appropriate isolated fractions were concentrated, filtered (to remove any remaining DCU), and directly precipitated in hexanes and cooled to −20° C. overnight. The hexanes were decanted and the precipitate was isolated to yield 7.173 g of a sticky solid (97% yield). ¹H NMR (400 MHz, CDCl₃): δ 1.65 (m, 16, —CH₂—CH₂—CH₂—CH₂—), 2.33 (m, 8, —CH₂—CH₂—CH₂—CH₂—), 2.42 (m, 8, —CH₂—CH₂—CH₂—CH₂—), 2.59 (m, 4, —CH₂—CH₂—), 4.11 (m, 12, —CH₂—CH—CH₂—), 4.24 (m, 12, —CH₂—CH—CH₂—), 4.67 (m, 4, —CH₂—CH—CH₂—), 5.20 (m, 2, —CH—CH—CH₂—), 5.51 (s, 4, CH), 7.33 (m, 12, arom. CH), 7.47 (m, 8, arom. CH). ¹³C NMR (100.6 MHz, CDCl₃): δ 173.41 (COOR), 172.92 (COOR), 171.48 (COOR), 138.02 (CH), 129.28 (CH), 128.49 (CH), 126.21 (CH), 101.38 (CH), 69.65 (CH), 69.27 (CH₂), 66.11 (CH), 62.19 (CH₂), 34.09 (CH₂), 33.73 (CH₂), 28.97 (CH₂), 24.44 (CH₂), 24.36 (CH₂). FAB MS 1425.5 m/z [M+H]⁺ (Theory: 1427.49 m/z [M]⁺). SEC M_{w}: 1670, Mₙ: 1650, PDI: 1.01.

### EXAMPLE 35

### Synthesis of [G0]-PGLSA-[G1]-PGLAA-OH

Synthesis of [G0]-PGLSA-[G1]-PGLAA-OH—Pd(OH)₂/C (10% w/w) was added to a solution of [G0]-PGLSA-[G1]-PGLAA-bzld (5.900 g, 4.133 mmol) in THF (50 mL). The flask for catalytic hydrogenolysis was evacuated and filled with 60 psi of H₂ before shaking for 10 hours. The catalyst was filtered and washed with THF (50 mL). The filtrate was evaporated to give 4.407 g of a colorless, viscous oil (99% yield). ¹H NMR (400 MHz, CD₃OD): δ 1.63 (m, 16, —CH₂—CH₂—CH₂—CH₂—), 2.36 (m, 16, —CH₂—CH₂—CH₂—CH₂—), 2.61 (m, 4, —CH₂—CH₂—), 3.52 (m, 3, —CH₂—CH—CH₂—), 3.59-3.65 (broad m, 9, —CH₂—CH—CH₂—), 3.69 (m, 2, —CH₂—CH—CH₂—), 3.79 (m, 2, —CH₂—CH—CH₂—), 4.03 (m, 2, —CH₂—CH—CH₂—), 4.15 (m, 5, —CH₂—CH—CH₂—), 4.30 (m, 4, —CH₂—CH—CH₂—), 5.25 (m, 2, —CH₂—CH—CH₂—). ¹³C NMR (100.6 MHz, CD₃OD): δ 173.85 (COOR), 173.67 (COOR), 173.41 (COOR), 171.95 (COOR), 75.42 (CH), 69.93 (CH), 69.78 (CH), 65.36 (CH₂), 62.87 (CH₂), 62.04 (CH₂), 60.50 (CH₂), 33.50 (CH₂), 33.29 (CH₂), 33.19 (CH₂), 28.61 (CH₂), 24.12 (CH₂). MALDI-TOF MS 1097.5 m/z [M+Na]⁺(Theory: 1075.06 m/z [M]⁺). SEC M_{w}: 1680, Mₙ: 1660, PDI: 1.01.

### EXAMPLE 36

### Synthesis of [G0]-PGLSA-[G1]-PGLAA-[G2]-PGLSA-bzld

Synthesis of [G0]-PGLSA-[G1]-PGLAA-[G2]-PGLSA-bzld—2-(cis-1,3-O-benzylidene glycerol)succinic acid mono ester (12.758 g, 45.520 mmol), [G0]-PGLSA-[G1]-PGLAA-OH (4.284 g, 3.984 mmol), and DPTS (5.112 g, 17.381 mmol) were dissolved in THF (100 mL) followed by the addition of DCC (13.912 g, 67.436 mmol). The reaction was stirred at room temperature for 14 hours under nitrogen atmosphere. Upon completion, the DCC-urea was filtered and washed with a small amount of THF (50 mL) and the solvent was evaporated. The crude product was purified by silica gel chromatography, eluting with 2% MeOH in DCM. The appropriate isolated fractions were concentrated, filtered (to remove any remaining DCU), and directly precipitated in hexanes and cooled to −20° C. overnight. The hexanes were decanted and the precipitate was isolated to yield 10.84 g of a white solid (85.7% yield). ¹H NMR (400 MHz, CDCl₃): δ 1.60 (m, 17, —CH₂—CH₂—CH₂—CH₂—), 2.30 (m, 17, —CH₂—CH₂H₂—CH₂—), 2.63 (m, 20, —CH₂—CH₂—), 2.72 (m, 16, —CH₂—CH₂—), 4.11 (m, 29, —CH₂—CH—CH₂—), 4.23 (m, 29, —CH₂—CH—CH₂—), 4.70 (m, 8, —CH₂—CH—CH₂—), 5.20 (m, 6, —CH₂—CH—CH₂—), 5.51 (s, 8, CH), 7.34 (m, 12, arom. CH), 7.46 (m, 8, arom. CH). ¹³C NMR (100.6 MHz, CDCl₃): δ 173.41 (COOR), 172.92 (COOR), 171.48 (COOR), 138.02 (CH), 129.28 (CH), 128.49 (CH), 126.21 (CH), 101.38 (CH), 69.65 (CH), 69.27 (CH₂), 66.11 (CH), 62.19 (CH₂), 34.09 (CH₂), 33.73 (CH₂), 28.97 (CH₂), 24.44 (CH₂), 24.36 (CH₂). MALDI-TOF MS 3172.7 m/z [M+Na]⁺(Theory:. 3173.13 m/z [M]⁺). SEC M_{w}: 3600, Mₙ: 3540, PDI: 1.02.

### EXAMPLE 37

### Synthesis of [G0]-PGLSA-[G1]-PGLAA-[G2]-PGLSA-OH

Synthesis of [G0]-PGLSA-[G1]-PGLAA-[G2]-PGLSA-OH—Pd(OH)₂/C (10% w/w) was added to a solution of [G0]-PGLSA-[G1]-PGLAA-[G2]-PGLSA-bzld (5.251 g, 1.655 mmol) in THF (100 mL). The flask for catalytic hydrogenolysis was evacuated and filled with 60 psi of H₂ before shaking for 10 hours. The catalyst was filtered and washed with THF (50 mL). The filtrate was evaporated to give 4.011 g of a colorless, viscous oil (98.2% yield). ¹H NMR (400 MHz, CD₃OD): δ 1.62 (m, 17, —CH₂—CH₂—CH₂—CH₂—), 2.36 (m, 17, —CH₂—CH₂—CH₂—CH₂—), 2.64 (m, 36, —CH₂—CH₂—), 3.52 (m, 2, —CH₂—CH—CH₂—), 3.60-3.66 (broad m, 26, —CH₂—CH—CH₂—), 3.69 (m, 9, —CH₂—CH—CH₂—), 3.80 (m, 1, —CH₂—CH—CH₂—), 4.18 (m, 14, —CH₂—CH—CH₂—), 4.32 (m, 12, —CH₂—CH—CH₂—), 5.25 (m, 6, —CH₂—CH—CH₂—). ³C NMR (100.6 MHz, CD₃OD): δ 173.38 (COOR), 173.05 (COOR), 172.56 (COOR), 172.24 (COOR), 172.00 (COOR), 75.81 (CH), 69.80 (CH), 69.35 (CH), 67.65 (CH₂), 65.68 (CH₂), 62.87 (CH₂), 62.42 (CH₂), 62.11 (CH₂), 60.43 (CH₂), 33.49 (CH₂), 33.20 (CH₂), 28.83 (CH₂), 28.64 (CH₂), 25.28 (CH₂), 24.09 (CH₂). MALDI-TOF MS 2492.0 m/z [M+Na]⁺ (Theory: 2468.27 m/z [M]⁺). SEC M_{w}: 3390, Mₙ: 3340, PDI: 1.02.

### EXAMPLE 38

### Synthesis of [G0]-PGLSA-[G1]-PGLAA-[G2]-PGLSA-[G3]-PGLAA-bzld

Synthesis of [G0]-PGLSA-[G1]-PGLAA-[G2]-PGLSA-[G3]-PGLAA-bzld—2-(cis-1,3-O-benzylidene glycerol)adipic acid mono ester (10.751 g, 34.869 mmol), [G0]-PGLSA-[G1]-PGLAA-[G2]-PGLSA-OH (3.771 g, 1.528 mmol), and DPTS (1.463 g, 4.975 mmol) were dissolved in THF (120 mL) followed by the addition of DCC (10.598 g, 51.365 mmol). The reaction was stirred at room temperature for 14 hours under nitrogen atmosphere. Upon completion, the DCC-urea was filtered and washed with a small amount of THF (50 mL) and the solvent was evaporated. The crude product was purified by silica gel chromatography, eluting with 1.5% MeOH in DCM. The appropriate isolated fractions were concentrated, filtered (to remove any remaining DCU), and directly precipitated in hexanes and cooled to −20° C. overnight. The hexanes were decanted and the precipitate was isolated to yield 9.88 g of a sticky solid (90.9% yield). ¹H NMR (400 MHz, CDCl₃): δ 1.65 (m, 81, —CH₂—CH₂—CH₂—CH₂—), 2.31 (m, 52, —CH₂—CH₂—CH₂—CH₂—), 2.42 (m, 32, —CH₂—CH₂—CH₂—CH₂—), 2.58 (m, 36 —CH₂—CH₂—), 4.10 (m, 62, —CH₂—CH—CH₂—), 4.23 (m, 62, —CH₂—CH—CH₂—), 4.66 (m, 16, —CH₂—CH—CH₂—), 5.19 (m, 14, —CH₂—CH—CH₂—), 5.51 (s, 16, CH), 7.33 (m, 47, arom. CH), 7.46 (m, 32, arom. CH). ¹³C NMR (100.6 MHz, CDCl₃): δ 173.39 (COOR), 172.90 (COOR), 171.82 (COOR), 171.53 (COOR), 138.04 (CH), 129.26 (CH), 128.49 (CH), 126.22 (CH), 101.36 (CH), 69.65 (CH), 69.26 (CH₂), 66.11 (CH), 62.64 (CH₂), 62.15 (CH₂), 34.07 (CH₂), 33.73 (CH₂), 28.96 (CH₂), 28.80 (CH₂), 24.43 (CH₂), 24.35 (CH₂). MALDI-TOF MS 7137.3 m/z [M+Na]⁺ (Theory: 7113.25 m/z [M]⁺). SEC M_{w}: 7160, Mₙ: 7060, PDI: 1.01.

### EXAMPLE 39

### Synthesis of [G0]-PGLSA-[G1]-PGLAA-[G2]-PGLSA-[G3]-PGLAA-OH

Synthesis of [G0]-PGLSA-[G1]-PGLAA-[G2]-PGLSA-[G3]-PGLAA-OH—Pd(OH)₂/C (10% w/w) was added to a solution of [G0]-PGLSA-[G1]-PGLAA-[G2]-PGLSA-[G3]-PGLAA-bzld (9.175 g, 1.290 mmol) in THF (100 mL). The flask for catalytic hydrogenolysis was evacuated and filled with 60 psi of H₂ before shaking for 10 hours. The catalyst was filtered and washed with THF (50 mL). The filtrate was evaporated to give 7.218 g of a colorless, viscous oil (98.1% yield). ¹H NMR (400 MHz, CD₃OD): δ 1.63 (m, 83, —CH₂—CH₂—CH₂—CH₂—), 2.37 (m, 83, —CH₂—CH₂—CH₂—CH₂—), 2.61 (m, 36, —CH₂—CH₂—), 3.52 (m, 8, —CH₂—CH—CH₂—), 3.60-3.71 (broad m, 57, —CH₂—CH—CH₂—), 3.80 (m, 4, —CH₂—CH—CH₂—), 4.03 (m, 5, —CH₂—CH—CH₂—), 4.11-4.23 (m, 34, —CH₂—CH—CH₂—), 4.30 (m, 29, —CH₂—CH—CH₂—), 5.25 (m, 14, —CH₂—CH—CH₂—). ¹³C NMR (100.6 MHz, CD₃OD): δ 173.85 (COOR), 173.67 (COOR), 173.41 (COOR), 171.95 (COOR), 75.42 (CH), 69.93 (CH), 69.78 (CH), 65.36 (CH₂), 62.87 (CH₂), 62.04 (CH₂), 60.50 (CH₂), 33.50 (CH₂), 33.29 (CH₂), 33.19 (CH₂), 28.61 (CH₂), 24.12 (CH₂). MALDI-TOF MS 5730.3 m/z [M+Na]⁺ (Theory: 5703.54 m/z [M]⁺). SEC M_{w}: 6570, Mₙ: 6490, PDI: 1.01.

### EXAMPLE 40

### Synthesis of [G0]-PGLSA-[G1]-PGLAA-[G2]-PGLSA-[G3]-PGLAA-[G4]-PGLSA-bzld

Synthesis of [G0]-PGLSA-[G1]-PGLAA-[G2]-PGLSA-[G3]-PGLAA-[G4]-PGLSA-bzld—2-(cis-1,3-O-benzylidene glycerol)succinic acid mono ester (11.572 g, 41.286 mmol), [G0]-PGLSA-[G1]-PGLAA-[G2]-PGLSA-[G3]-PGLAA-OH (5.593 g, 0.981 mmol), and DPTS (4.094 g, 13.919 mmol) were dissolved in THF (80 mL) followed by the addition of DCC (12.596 g, 61.048 mmol). The reaction was stirred at room temperature for 14 hours under nitrogen atmosphere. Upon completion, the DCC-urea was filtered and washed with a small amount of THF (50 mL) and the solvent was evaporated. The crude product was purified by silica gel chromatography, eluting with 1.5% to 5.0% MeOH in DCM. The appropriate isolated fractions were concentrated, filtered (to remove any remaining DCU), and directly precipitated in hexanes and cooled to −20° C. over 48 hours. The hexanes were decanted and the precipitate was isolated to yield 11.50 g of a white solid (83.2% yield). ¹H NMR (400 M}{z, CDCl₃): δ 1.59 (m, 83, —CH₂—CH₂—CH₂—CH₂—), 2.30 (m, 83, —CH₂—CH₂—CH₂—CH₂—), 2.62 (m, 104, —CH₂—CH₂—), 2.70 (m, 63, —CH₂—CH₂—), 4.12 (m, 130, —CH₂—CH—CH₂—), 4.22 (m, 130, —CH₂—CH—CH₂—), 4.68 (m, 32, —CH₂—CH—CH₂—), 5.18 (m, 30, —CH₂—CH—CH₂—), 5.50 (s, 32, CH), 7.33 (m, 97, arom. CH), 7.46 (m, 66, arom. CH). ¹³C NMR (100.6 MHz, CDCl₃): δ 172.88 (COOR), 172.53 (COOR), 172.25 (COOR), 171.89 (COOR), 138.04 (CH), 129.26 (CH), 128.48 (CH), 126.22 (CH), 101.28 (CH), 69.14 (CH₂), 66.54 (CH), 62.60 (CH₂), 33.81 (CH₂), 33.66 (CH₂), 29.35 (CH₂), 29.03 (CH₃), 24.30 (CH₂). SEC M_{w}: 10440, Mₙ: 10290, PDI: 1.02.

### EXAMPLE 41

### Synthesis of [G0]-PGLSA-[G1]-PGLAA-[G2]-PGLSA-[G3]-PGLAA-[G4]-PGLSA-OH

Synthesis of [G0]-PGLSA-[G1]-PGLAA-[G2]-PGLSA-[G3]-PGLAA-[G4]-PGLSA-OH—Pd(OH)₂/C (10% w/w) was added to a solution of [G0]-PGLSA-[G1]-PGLAA-[G2]-PGLSA-[G3]-PGLAA-[G4]-PGLSA-bzld (2.084 g, 0.1478 mmol) in THF (80 mL). The flask for catalytic hydrogenolysis was evacuated and filled with 60 psi of H₂ before shaking for 10 hours. The catalyst was filtered and washed with THF (75 mL). The filtrate was evaporated to give 1.652 g of a colorless, viscous oil (99.1% yield). ¹H NMR (400 MHz, CD₃OD): δ 1.62 (m, 80, —CH₂—CH₂—CH₂—CH₂—), 2.37 (m, 80, —CH₂—CH₂—CH₂—CH₂—), 2.64 (m, 164, —CH₂—CH₂—), 3.52 (m, 12, —CH₂—CH—CH₂—), 3.63-3.71 (broad m, 160, —CH₂—CH—CH₂—), 3.80 (m, 6, —CH₂—CH—CH₂—), 4.06 (m, 14, —CH₂—CH—CH₂—), 4.20 (m, 62, —CH₂—CH—CH₂—), 4.30 (m, 60, —CH₂—CH—CH₂—), 5.25 (m, 30, —CH₂—CH—CH₂—). ¹³C NMR (100.6 MHz, CD₃OD): δ 173.40 (COOR), 173.06 (COOR), 172.58 (COOR), 75.82 (CH), 69.90 (CH), 69.34 (CH), 67.64 (CH₂), 62.45 (CH₂), 62.15 (CH₂), 60.46 (CH₂), 33.25 (CH₂), 28.87 (CH₂), 28.67 (CH₂), 25.27 (CH₂), 24.12 (CH₂). MALDI-TOF MS 11299.1 m/z [M+Na]⁺ (Theory: 11276.39 m/z [M]⁺). SEC M_{w}: 9150, Mₙ: 9000, PDI: 1.02.

### EXAMPLE 42

Synthesis of PEG-([G0]-PGLSA-bzld)₂—This example is shown for PEG of 3400 Mw, but we have also used PEG of 10,000 and 20,000 Mw. PEG, Mₙ=3400, (10.0 g, 2.94 mmol), which was dried under vacuum at 120° C. for three hours, and [2-(cis-1,3-O-benzylidene glycerol)-N-succinimidyl] succinate (4.03 g, 10.7 mmol) were dissolved in CH₂Cl₂ (100 mL) and stirred under nitrogen. TEA (2.0 mL, 14 mmol) was added by syringe and stirring was continued for 14 hours. Any remaining activated ester was quenched by the addition of fresh TEA (1.0 mL, 7.2 mmol) and n-propanol (1.0 mL, 11 mmol), which was allowed to stir for another 10 hours. After removing most of the solvent, the product was precipitated in cold ethyl ether (700 mL) and collected to yield 11.1 g of a white solid (97% yield). ¹H NMR obtained. Elemental Analysis C: 55.31%; H 8.58% (Theory C: 55.56%; H 8.66.%). MALDI MS M_{w}: 4020, Mₙ: 3940, PDI: 1.02. SEC M_{w}: 3980, Mₙ: 3950, PDI: 1.03.

### EXAMPLE 43

Synthesis of PEG-([G0]-PGLSA-OH)₂—Pd/C (10% w/w) was added to a solution of PEG-([G0]-PGLSA-bzld)₂ (5.07 g, 1.29 mmol) in 80 mL of 9:1 ethyl acetate/methanol. The apparatus for catalytic hydrogenolysis was evacuated and filled with 50 psi of H₂ before shaking for 8 hours. The catalyst was filtered off and washed with ethyl acetate (20 mL). The filtrate was evaporated and the remaining white solid was redissolved in a minimal amount of CH₂Cl₂ (15 mL)and precipitated in cold ethyl ether (600 mL) to give 4.52 g of a white solid (93% yield). ¹H NMR obtained. Elemental Analysis C: 53.49%; H 8.78% (Theory C: 53.69%; H 8.85%). MALDI MS M_{w}: 3780, Mₙ: 3730, PDI: 1.01. SEC M_{w}: 3860, Mₙ: 3710, PDI: 1.021.

### EXAMPLE 44

Synthesis of PEG-([G1]-PGLSA-bzld)₂—PEG-([G0]-PGLSA-OH)₂ (5.81 g, 1.55 mmol), which was dried under vacuum at 80° C. for three hours, and [2-(cis-1,3-O-benzylidene glycerol)-N-succinimidyl] succinate (4.35 g, 11.5 mmol) were dissolved in CH₂Cl₂ (70 mL) and stirred under nitrogen. TEA (1.75 mL, 13.0 mmol) was added by syringe and stirring was continued for 14 hours. Any remaining activated ester was quenched by the addition of fresh TEA (1.0 mL, 7.2 mmol) and n-propanol (1.0 mL, 11 mmol), which was allowed to stir for another 10 hours. After removing most of the solvent, the product was precipitated in cold ethyl ether (700 mL) and collected to yield 7.15 g (96% yield). ¹H NMR obtained. MALDI MS M_{w}: 4520, Mₙ: 4480, PDI: 1.01. SEC M_{w}: 4420, Mₙ: 4240, PDI: 1.04.

### EXAMPLE 45

Synthesis of PEG-([G1]-PGLSA-OH)₂—Pd/C (10% w/w) was added to a solution of PEG-([G1]-PGLSA-bzld)₂ (5.53 g, 1.15 mmol) in 80 mL of 9:1 ethyl acetate/methanol. The apparatus for catalytic hydrogenolysis was evacuated and filled with 50 psi of H₂ before shaking for 8 hours. The catalyst was filtered off and washed with ethyl acetate (20 mL). The filtrate was evaporated and the remaining white solid was redissolved in a minimal amount of CH₂Cl₂ (15 mL) and precipitated in cold ethyl ether (700 mL) to give 4.71 g of a white solid (92% yield). ¹H NMR obtained. MALDI MS M_{w}: 4320, Mₙ: 4280, PDI: 1.01. SEC M_{w}: 4390, Mₙ: 4230, PDI: 1.04.

### EXAMPLE 46

Synthesis of PEG-([G1]-PGLSA-MA)₂—PEG-([G1]-PGLSA-OH)₂ (1.03 g, 0.232 mmol), which was dried under vacuum at 80° C. for three hours, was dissolved in CH₂Cl₂ (40 mL) and stirred under nitrogen before the addition of methacryloyl chloride (1.93 g, 5.12 mmol). TEA (0.80 mL, 5.74 mmol) was added by syringe and stirring was continued for 14 hours. The mixture was diluted with more CH₂Cl₂ (60 mL) and washed twice with 0.1 N HCl (100 mL). After drying with Na₂SO₄, filtering, and removing most of the solvent, the product was precipitated in cold ethyl ether and collected to yield 1.08 g (94% yield). ¹H NMR obtained. SEC M_{w}: 4610, Mₙ: 4420, PDI: 1.04.

### EXAMPLE 47

Synthesis of PEG-([G2]-PGLSA-bzld)₂—PEG-([G1]-PGLSA-OH)₂ (0.697 g, 0.150 mmol), which was dried under vacuum at 80° C. for three hours, and [2-(cis-1,3-O-benzylidene glycerol)-N-succinimidyl] succinate (1.01 g, 2.68 mmol) were dissolved in CH₂Cl₂ (30 mL) and stirred under nitrogen. TEA (0.50 mL, 3.59 mmol) was added by syringe and stirring was continued for 14 hours. Any remaining activated ester was quenched by the addition of fresh TEA (1.0 mL, 7.2 mmol) and n-propanol (1.0 mL, 11 mmol), which was allowed to stir for another 10 hours. After removing most of the solvent, the product was precipitated in cold ethyl ether (400 mL) and collected to yield 0.940 g (93% yield). ¹H NMR obtained.

### EXAMPLE 48

Synthesis of ([G1]-PGLSA-MA)₂—PEG (8)-([G1]-PGLSA-OH)₂—PEG (0.500 g, 0.113 mmol) was dissolved in DCM (15 mL) and stirred under nitrogen before methacrylic anhydride (0.56 mL, 3.76 mmol) was added by syringe. DMAP (86.0 mg, 0.704 mmol) was added and stirring was continued for 14 hours. Any remaining anhydride was quenched by the addition of methanol (0.1 mL, 3.95 mmol), which was allowed to stir for another 5 hours. The reaction was diluted with DCM (35 mL) and washed with 0.1 N HCl (50 mL) and brine (50 mL). The organic phase was dried with Na₂SO₄ and filtered before the PEG-based dendrimer was precipitated in cold (−20° C.) ethyl ether (300 mL) and collected to yield 0.519 g of a white solid (93% yield). ¹H NMR (CDCl₃): _{—}1.90 (m, 19, —CH₃), 2.61 (m, 21, —CH₂—CH₂—), 3.42 (t, 2, —CH₂—CH₂—), 3.55-3.65 (broad m, 285, —CH₂—CH₂—), 3.77 (t, 2, —CH₂—CH₂—), 4.09-4.37 (broad m, 29, —CH₂—CH—CH₂—), 5.22 (m, 2, —CH₂—CH—CH₂—), 5.35 (m, 2, —CH₂—CH—CH₂—), 5.57 (m, 6, CH), 6.07 (m, 6, CH). ¹³C NMR (CDCl₃): _{—}171.89 (COOR), 135.84 (CH), 126.64 (CH), 70.75 (CH₂), 69.45 (CH), 62.61 (CH₂), 28.87 (CH₂), 18.43 (CH₃). FTIR: _(cm⁻¹) 2873 (aliph. C—H stretch), 1736 (C═O). MALDI MS M_{w}: 5012, Mₙ: 4897, PDI: 1.02. SEC M_{w}: 3910, Mₙ: 3740, PDI: 1.04. Tₘ=40.8.

### EXAMPLE 49

Synthesis of ([G2]-PGLSA-bzld)₂—PEG (9)—([G1]-PGLSA-OH)₂—PEG (3.25 g, 0.737 mmol), and 2-(cis-1,3-O-benzylidene glycerol)succinic acid mono ester anhydride (12.68 g, 23.37 mmol) were dissolved in DCM (50 mL) and stirred under nitrogen. DMAP (0.588 g, 4.81 mmol) was added and stirring was continued for 14 hours. Any remaining anhydride was quenched by the addition of n-propanol (2.5 mL, 28 mmol), which was allowed to stir for another 5 hours. The reaction was diluted with DCM (50 mL) and washed with 0.1 N HCl (100 mL), saturated sodium bicarbonate (100 mL 3×), and brine (100 mL). The organic phase was dried with Na₂SO₄, filtered, and concentrated before the PEG-based dendrimer was precipitated in cold (−20° C.) ethyl ether (400 mL) and collected to yield 4.57 g of a white solid (91% yield). ¹H NMR (CDCl₃): _{—}2.61 (broad m, 40, —CH₂—CH₂—), 2.72 (broad m, 16, —CH₂—CH₂—), 3.43 (t, 2, —CH₂—CH₂—), 3.55-3.65 (broad m, 280, —CH₂—CH₂—), 3.77 (t, 2, —CH₂—CH₂—), 4.13 (broad m, 28, —CH₂—CH—CH₂—), 4.22 (broad m, 28, —CH₂—CH—CH₂—), 4.69 (m, 8, —CH₂—CH—CH₂—), 5.20 (m, 6, —CH₂—CH—CH₂—), 5.50 (s, 8, CH), 7.32 (m, 24, arom. CH), 7.46 (m, 16, arom. CH). ¹³C NMR (CDCl₃): _{—}172.28 (COOR), 171.91 (COOR), 171.57 (COOR), 138.01 (CH), 129.26 (CH), 128.48 (CH), 126.21 (CH), 101.33 (CH), 70.56 (CH₂), 69.50 (CH), 69.16 (CH₂), 66.53 (CH), 64.08 (CH₂), 29.49 (CH₂), 29.21 (CH₂). FTIR: _(cm⁻¹) 2879(aliph. C—H stretch), 1736 (C═O). MALDI MS M_{w}: 6642, Mₙ: 6492, PDI: 1.02. SEC M_{w}: 4860, Mₙ: 4680, PDI: 1.04. Tₘ=31.4.

### EXAMPLE 50

Synthesis of ([G2]-PGLSA-OH)₂—PEG (10)—Pd(OH)₂/C (10% w/w) was added to a solution of ([G2]-PGLSA-bzld)₂—PEG (3.26 g, 0.500 mmol) in 25 mL of 2:1 DCM/methanol. The apparatus for catalytic hydrogenolysis was evacuated and filled with 60 psi of H₂ before shaking for 8 hours. The catalyst was filtered off and washed with DCM (20 mL). The PEG-based dendrimer was isolated after evaporation of solvents to give 2.86 g of a white solid (98% yield).

¹H NMR (CDCl₃): _{—}2.63 (broad m, 56, —CH₂—CH₂—), 3.42 (s, 4, —CH₂—CH₂—), 3.50-3.67 (broad m, 285, —CH₂—CH₂—), 3.72 (broad m, 27, —CH₂—CH—CH₂—), 4.14-4.29 (broad m, 32, —CH₂—CH—CH₂—), 4.88 (m, 8, —CH₂—CH—CH₂—), 5.22 (m, 6, —CH₂—CH—CH₂—). ¹³C NMR (CDCl₃): _{—}172.56 (COOR), 172.32 (COOR), 76.01 (CH), 70.78 (CH₂), 69.56 (CH), 69.22 (CH₂), 64.14 (CH₂), 63.52 (CH₂), 62.60 (CH₂), 61.93 (CH₂), 29.44 (CH₂), 29.21 (CH₂), 28.98 (CH₂). FTIR: _(cm⁻¹) 3452 (OH), 288. (aliph. C—H stretch), 1735 (C═O). MALDI MS M_{w}: 5910, Mₙ: 5788, PDI: 1.02. SEC M_{w}: 5340, Mₙ: 5210, PDI: 1.03. Tₘ=36.5.

### EXAMPLE 51

Synthesis of ([G2]-PGLSA-MA)₂—PEG (11)—([G2]-PGLSA-OH)₂—PEG (0.501 g, 0.0863 mmol) was dissolved in DCM (15 mL) and stirred under nitrogen before methacrylic anhydride (0.50 mL, 3.36 mmol) was added by syringe. DMAP (72.1 mg, 0.990 mmol) was added and stirring was continued for 14 hours. Any remaining anhydride was quenched by the addition of methanol (0.1 mL, 3.95 mmol), which was allowed to stir for another 5 hours. The reaction was diluted with DCM (35 mL) and washed with 0.1 N HCl (50 mL) and brine (50 mL). The organic phase was dried with Na₂SO₄ and filtered before the PEG-based dendrimer was precipitated in cold (−20° C.) ethyl ether (300 mL) and collected to yield 0.534 g of a white solid (90% yield). ¹H NMR (CDCl₃): _{—}1.89 (m, 47, —CH₃), 2.60 (m, 65, —CH₂—CH₂—), 3.56-3.67 (broad m, 387, —CH₂—CH₂—), 3.77 (t, 2, —CH₂—CH₂—), 4.12-4.37 (broad m, 81, —CH₂—CH—CH₂—), 5.21 (m, 13, —CH₂—CH—CH₂—), 5.33 (m, 7, —CH₂—CH—CH₂—), 5.56 (m, 16, CH), 6.06 (m, 16, CH).

¹³C NMR (CDCl₃): _{—}171.89 (COOR), 135.84 (CH), 126.64 (CH), 70.75 (CH₂), 69.45 (CH), 62.61 (CH₂), 28.87 (CH₂), 18.43 (CH₃). FTIR: _(cm⁻¹) 2873 (aliph. C—H stretch), 1736 (C═O). %). MALDI MS M_{w}: 6956, Mₙ: 6792, PDI: 1.02. SEC M_{w}: 4580, Mₙ: 4390, PDI: 1.04. Tₘ=27.0.

### EXAMPLE 52

Synthesis of ([G3]-PGLSA-bzld)₂—PEG (12)—([G2]-PGLSA-OH)₂—PEG (2.13 g, 0.367 mmol), and 2-(cis-1,3-O-benzylidene glycerol)succinic acid mono ester anhydride (12.71 g, 23.43 mmol) were dissolved in DCM (45 mL) and stirred under nitrogen. DMAP (0.608 g, 4.98 mmol) was added and stirring was continued for 14 hours. Any remaining anhydride was quenched by the addition of n-propanol (2.0 mL, 22 mmol), which was allowed to stir for another 5 hours. The reaction was diluted with DCM (55 mL) and washed with 0.1 N HCl (100 mL), saturated sodium bicarbonate (100 mL 3×), and brine (100 mL). The organic phase was dried with Na₂SO₄, filtered, and concentrated before the PEG-based dendrimer was precipitated in cold (−20° C.) ethyl ether (400 mL) overnight and collected to yield 3.35 g of a white solid (92% yield). ¹H NMR (CDCl₃): _{—}2.61 (broad m, 84, —CH₂—CH₂—), 2.74 (broad m, 36, —CH₂—CH₂—), 3.43 (t, 2, —CH₂—CH₂—), 3.56-3.65 (broad m, 278, —CH₂—CH₂—), 3.78 (t, 2, —CH₂—CH₂—), 4.13 (broad m, 60, —CH₂—CH—CH₂—), 4.21 (broad m, 60, —CH₂—CH—CH₂—), 4.69 (m, 16, —CH₂—CH—CH₂—), 5.19 (m, 14, —CH₂—CH—CH₂—), 5.50 (s, 16, CH), 7.32 (m, 46, arom. CH), 7.46 (m, 30, arom. CH). ¹³C NMR (CDCl₃): _{—}172.28 (COOR), 171.91 (COOR), 138.03 (CH), 129.26 (CH), 128.48 (CH), 126.21 (CH), 101.31 (CH), 70.76 (CH₂), 69.49 (CH), 69.16 (CH₂), 66.53 (CH), 62.47 (CH₂), 29.35 (CH₂), 29.02 (CH₂), 28.83 (CH₂). FTIR: _(cm⁻¹) 2868 (aliph. C—H stretch), 1735 (C═O). MALDI MS M_{w}: 10215, Mₙ: 9985, PDI: 1.02. SEC M_{w}: 7020, Mₙ: 6900, PDI: 1.02. T_{g}=−13.6.

### EXAMPLE 53

Synthesis of ([G3]-PGLSA-OH)₂—PEG (13)—Pd(OH)₂/C (10% w/w) was added to a solution of ([G3]-PGLSA-bzld)₂—PEG (2.88 g, 0.288 mmol) in 30 mL of 2:1 DCM/methanol. The apparatus for catalytic hydrogenolysis was evacuated and filled with 60 psi of H₂ before shaking for 8 hours. The catalyst was filtered off and washed with DCM (20 mL). The PEG-based dendrimer was isolated after evaporation of solvents to give 2.86 g of a white solid (98% yield).

¹H NMR ((CD₃)₂CO):_{—}2.64 (broad m, 120, —CH₂—CH₂—), 3.49-3.60 (broad m, 286, —CH₂—CH₂—), 3.64-3.75 (broad m, 33, —CH₂—CH—CH₂—), 4.00-4.12 (broad m, 42, —CH₂—CH—CH₂—), 4.13-4.29 (broad m, 68, —CH₂—CH—CH₂—), 4.64 (t, 2, —CH₂—CH—CH₂—), 4.85 (t, 2, —CH₂—CH—CH₂—), 5.26 (m, 14, —CH₂—CH—CH₂—). ¹³C NMR ((CD₃)₂CO): _{—}171.85 (COOR), 171.64 (COOR), 76.09 (CH), 73.70 (CH₂), 70.56 (CH), 69.52 (CH₂), 66.19 (CH), 63.87 (CH₂), 62.31 (CH₂), 61.65 (CH₂), 60.69 (CH₂). FTIR: _(cm⁻¹) 3432 (OH), 2925 (aliph. C—H stretch), 1734 (C═O). MALDI MS M_{w}: 8765, Mₙ: 8575, PDI: 1.02. SEC M_{w}: 8090, Mₙ: 7820, PDI: 1.03. T_{g}=−38.2.

### EXAMPLE 54

Synthesis of ([G3]-PGLSA-MA)₂—PEG (14)—([G3]-PGLSA-OH)₂—PEG (0.223 g, 0.0260 mmol) was dissolved in THF (15 mL) and stirred under nitrogen before methacrylic anhydride (1.10 mL, 7.38 mmol) was added by syringe. DMAP (90.0 mg, 0.737 mmol) was added and stirring was continued for 14 hours. Any remaining anhydride was quenched by the addition of methanol (0.2 mL, 7.89 mmol), which was allowed to stir for another 5 hours. The reaction was diluted with DCM (35 mL) and washed with 0.1 N HCl (50 mL) and brine (50 mL). The organic phase was dried with Na₂SO₄ and filtered before the PEG-based dendrimer was precipitated in cold (−20° C.) ethyl ether (300 mL) and collected to yield 0.248 g of a white solid (89% yield). ¹H NMR (CDCl₃): _{—}1.90 (m, 76, —CH₃), 2.62 (m, 111, —CH₂—CH₂—), 3.56-3.67 (broad m, 285, —CH₂—CH₂—), 4.14-4.38 (broad m, 114, —CH₂—CH—CH₂—), 5.23 (m, 13, —CH₂—CH—CH₂—), 5.35 (m, 10, —CH₂—CH—CH₂—), 5.56 (m, 25, CH), 6.07 (m, 25, CH). ¹³C NMR (CDCl₃): _{—}171.87 (COOR), 135.91 (CH), 126.71 (CH), 70.76 (CH₂), 69.47 (CH), 62.62 (CH₂), 28.88 (CH₂), 18.43 (CH₃). FTIR: _(cm⁻¹) 2874 (aliph. C—H stretch), 1734 (C═O). MALDI MS M_{w}: 10722, Mₙ: 10498, PDI: 1.02. SEC M_{w}: 7000, Mₙ: 6820, PDI: 1.03. T_{g}=−37.9.

### EXAMPLE 55

Synthesis of ([G4]-PGLSA-bzld)₂—PEG—([G3]-PGLSA-OH)₂—PEG (1.82 g, 0.212 mmol), and 2-(cis-1,3-O-benzylidene glycerol)succinic acid mono ester anhydride (15.93 g, 29.36 mmol) were dissolved in THF (50 mL) and stirred under nitrogen. DMAP (0.537 g, 4.40 mmol) was added and stirring was continued for 14 hours. Any remaining anhydride was quenched by the addition of n-propanol (2.5 mL, 28 mmol), which was allowed to stir for another 5 hours. The reaction was diluted with DCM (50 mL) and washed with 0.1 N HCl (100 mL), saturated sodium bicarbonate (100 mL 3×), and brine (100 mL). The organic phase was dried with Na₂SO₄, filtered, and concentrated before the PEG-based dendrimer was precipitated in ethyl ether (400 mL) and collected to yield 3.11 g of a white solid (87% yield). ¹H NMR (CDCl₃): _{—}2.61 (broad m, 180, —CH₂—CH₂—), 2.70 (broad m, 64, —CH₂—CH₂—), 3.43 (t, 2, —CH₂—CH₂—), 3.56-3.65 (broad m, 286, —CH₂—CH₂—), 3.78 (t, 2, —CH₂—CH₂—), 4.11 (broad m, 125, —CH₂—CH—CH₂—), 4.23 (broad m, 125, —CH₂—CH—CH₂—), 4.68 (m, 32, —CH₂—CH—CH₂—), 5.20 (m, 30, —CH₂—CH—CH₂—), 5.49 (s, 32, CH), 7.32 (m, 93, arom. CH), 7.46 (m, 62, arom. CH). ¹³C NMR (CDCl₃): _{—}172.28 (COOR), 171.90 (COOR), 171.60 (COOR), 138.04 (CH), 129.26 (CH), 128.48 (CH), 126.21 (CH), 101.29 (CH), 70.76 (CH₂), 69.46 (CH), 69.15 (CH₂), 66.53 (CH), 62.57 (CH₂), 29.34 (CH₂), 29.18 (CH₂), 29.02 (CH₂), 28.83 (CH₂). FTIR: _(cm⁻¹) 2865 (aliph. C—H stretch), 1734 (C═O). MALDI MS M_{w}: 17289, Mₙ: 16968, PDI: 1.02. SEC M_{w}: 8110, Mₙ: 7950, PDI: 1.02. T_{g}=5.3.

### EXAMPLE 56

Synthesis of ([G4]-PGLSA-OH)₂—PEG—Pd(OH)₂/C (10% w/w) was added to a solution of ([G4]-PGLSA-bzld)₂—PEG (2.88 g, 0.170 mmol) in 30 mL of 2:1 DCM/methanol. The apparatus for catalytic hydrogenolysis was evacuated and filled with 60 psi of H₂ before shaking for 8 hours. The catalyst was filtered off and washed with DCM (20 mL). The PEG-based dendrimer was isolated after evaporation of solvents to give 2.86 g of a white solid (98% yield).

¹H NMR ((CD₃)₂CO):_{—}2.64 (broad m, 248, —CH₂—CH₂—), 3.49-3.60 (broad m, 296, —CH₂—CH₂—), 3.66 (broad m, 50, —CH₂—CH—CH₂—), 3.82 (broad m, 42, —CH₂—CH—CH₂—), 4.04-4.16 (broad m, 66, —CH₂—CH—CH₂—), 4.28 (broad m, 124, —CH₂—CH—CH₂—), 4.86 (m, 10, —CH₂—CH—CH₂—), 5.27 (m, 30, —CH₂—CH—CH₂—). ¹³C NMR ((CD₃)₂CO): _{—}172.20 (COOR), 70.45 (CH₂), 70.10 (CH), 69.92 (CH₂), 65.96 (CH), 62.31 (CH₂). FTIR: _(cm⁻¹) 3445 (OH), 2931 (aliph. C—H stretch), 1713 (C═O). MALDI MS M_{w}: 14402, Mₙ: 14146, PDI: 1.02. SEC M_{w}: 9130, Mₙ: 8980, PDI: 1.02. T_{g}=−18.0.

### EXAMPLE 57

### Synthesis of bzld-[G1]-PGLSA-TBDPS

4.00 g (0.014 mol) of bzld-[G1]-PGLSA-CO₂H and 3.24 g (0.048 mol) of imidazole were stirred in 15 mL of DMF. Next, 6.4 mL (0.024 mol) of diphenyl-t-butyl silyl chloride were added and the reaction was stirred at 25° C. for 48 hours. The DMF was removed, the product was dissolved in CH₂Cl₂, washed with sat. NaHCO₃ and water, dried over Na₂SO₄, filtered, rotovapped, and dried on the vacuum line. The product was purified by column chromatography (4:1 hexanes:EtOAc) affording 6.38 g of product as a viscous opaque oil (86% yield). R_{f}=0.13 in 4:1 hexanes:EtOAc. ¹H NMR (CDCl₃): δ 1.09 (s, 9H, t-butyl), 2.78-2.84 (m, 4H, —CH₂—CH₂), 4.11-4.15 (m, 2H, —CH₂—CH—CH₂—), 4.23-4.26 (m, 2H, —CH₂—CH—CH₂—), 4.70-4.71 (m, 1H, —CH₂—CH—CH₂—), 5.54 (s, 1H, CH), 7.33-7.42, 7.48-7.50, 7.67-7.68 (m, 15H, arom. bzld and phenyl CH) ppm. ¹³C NMR (CDCl₃): δ 19.34 (—C—(CH₃)₃), 27.07 (—C—(CH₃)₃), 29.72, 30.96 (succ. —CH₂—), 66.46, 69.18 (glycerol, 2C, —CH₂—), 101.39 (O—CH—O), 126.23, 127.94, 128.50, 129.28, 130.29, 131.93, 135.51 (arom. CH), 137.99 (arom. bzld —C—), 171.53, 172.52 (succ. —C(═O)—) ppm. GC-MS: 519.2 m/z (MH⁺) (theory: 518.2 m/z (M⁺)). HR-FAB: 517.2028 m/z (M−H⁺) (theory: 518.2125 m/z (M⁺)). Elemental analysis: C, 69.18%; H, 6.69% (theory: C, 69.47%; H, 6.61%).

### EXAMPLE 58

### Synthesis of HO-[G1]-PGLSA-TBDPS

2.41 g (4.65 mmol) of bzld-[G1]-PGLSA-TBDPS was dissolved in 45 mL of THF, and 1.0 g of 20% Pd(OH)₂/C was added. The solution was then placed in a Parr tube on a hydrogenator, evacuated, flushed with hydrogen, and shaken under 50 psi H₂ for 3 hours. The solution was then filtered over wet celite. The product was purified by column chromatography (1:1 Hex:EtOAc increasing to 1:4 Hex:EtOAc) to yield 1.9 g of a clear oil (95% yield). ¹H NMR (CDCl₃): δ 1.08 (s, 9H, t-butyl), 2.02 (b s, 2H, —OH), 2.64-2.85 (m, 4H, —CH₂—CH₂), 3.70-3.72, 4.07-4.14 (m, 4H, —CH₂—CH—CH₂—), 4.83-4.86 (m, 1H, —CH₂—CH—CH₂—), 7.33-7.44, 7.62-7.65 (m, 10H, arom. phenyl CH) ppm. ¹³C NMR (CDCl₃): δ 19.30 (—C—(CH₃)₃), 27.03 (—C—(CH₃)₃), 29.77, 31.37 (succ. —CH₂—), 62.45 (glycerol, —CH₂—), 75.86 (CH₂—CH—CH₂), 127.97, 130.36, 132.67, 135.49 (phenyl CH), 172.65, 178.24 (succ. —C(═O)—) ppm. FAB-MS: 431 m/z (M−H⁺) (theory: 430.57 m/z (M⁺)).

Acetyl Derivative of Compound HO-[G1]-PGLSA-TBDPS:

Compound HO-[G1]-PGLSA-TBDPS was a hydroscopic oil and repeated attempts to obtain satisfactory EA failed. Thus, we decided to prepare the acetyl analog for elemental analysis. 0.44 g (1.02 mmol) of HO-[G1]-PGLSA-TBDPS was stirred in 30 mL of CH₂Cl₂ with 0.30 g (1.02 mmol) of DPTS, 0.15 mL (2.66 mmol) of freshly distilled acetic acid, and 0.63 g (3.07 mmol) of DCC. The solution was stirred at RT for 18 hours. The DCU precipitate was filtered and the solution was evaporated. A solution of 1:1 ethyl acetate:hexanes was added and impurities precipitated. The solution was filtered, concentrated and further purified by column chromatography (3:1 hexanes:EtOAc), to afford 0.44 g of product (83% yield). R_{f}=0.19 (4:1 hexanes:EtOAc)

¹H NMR (CDCl₃): δ 1.08 (s, 9H, t-butyl), 1.87-1.93 (m, 6H, —CH₃), 2.50-2.71 (m, 4H, —CH₂—CH₂), 3.96-4.19 (m, 4H, —CH₂—CH—CH₂—), 5.06-5.18 (m, 1H, —CH₂—CH—CH₂—), 7.22-7.33, 7.51-7.56 (m, 10H, phenyl CH) ppm. ¹³C NMR (CDCl₃): δ 19.10 (—C—(CH₃)₃), 20.61 (OC—CH₃), 26.82 (—C—(CH₃)₃), 29.14, 30.62 (succ. —CH₂—), 62.12, 69.28 (glycerol, —CH₂—), 127.71, 130.09, 131.65, 135.27 (arom. CH), 170.52, 171.19, 171.58 (—C(═O)—) ppm. FAB-MS: 515.4 m/z (MH⁺) (theory: 514.6 m/z (M⁺)). Elemental analysis: C, 62.76%; H, 6.69% (theory: C, 63.01%; H, 6.66%). SEC: M_{w}=547, Mₙ=528, PDI=1.04.

### EXAMPLE 59

### Synthesis of bzld-[G2]-PGLSA-TBDPS

1.90 g (4.41 mmol) of HO-[G1]-PGLSA-TBDPS was stirred in 100 mL of CH₂Cl₂ with 1.30 g (1 equiv; 4.41 mmol) of DPTS, 2.72 g (9.70 mmol; 2.2 equiv) of 2(cis-1,3-O-benzylidene glycerol)succinic acid monoester, and 2.00 g (9.70 mmol; 2.2 equiv) of DCC. The solution was stirred at RT for 18 hours. The DCU precipitate was filtered off and the solution was evaporated. A solution of 1:1 ethyl acetate:hexanes was added and impurities precipitated. The solution was filtered, concentrated and further purified by column chromatography (1:1 hexanes:EtOAc) to afford 3.70 g of product (88% yield). R_{f}=0.216 (1:1 hexanes:EtOAc). ¹H NMR (CDCl₃): δ 1.08 (s, 9H, t-butyl), 2.57-2.79 (m, 12H, —CH₂—CH₂), 4.08-4.14, 4.16-4.22 (m, 12H, —CH₂—CH—CH₂—), 4.70-4.71 (m, 2H, —CH₂—CH—CH₂—), 5.21 (m, 1H, CH), 5.49-5.54 (m, 1H, CH), 7.32-7.41, 7.47-7.49, 7.64-7.58 (m, 20H, arom. bzld and phenyl CH) ppm. ¹³C NMR (CDCl₃): δ 19.31 (—C—(CH₃)₃), 27.04 (—C—(CH₃)₃), 28.98, 29.33, 30.81 (succ. —CH₂—), 62.48, 66.50, 69.16, 69.43 (glycerol, —CH₂—), 101.33 (O—CH—O), 126.22, 127.95, 128.49, 129.26, 130.32, 131.92, 135.49 (arom. CH), 138.02 (arom. bzld —C—), 171.93, 172.28 (succ. —C(═O)—) ppm. GC−MS: 955.3 m/z (MH⁺) (theory: 954.4 m/z (M⁺)). Elemental analysis: C, 64.35%; H, 6.29% (theory: C, 64.14%; H, 6.12%). SEC: M_{w}=940, Mₙ=930, PDI=1.01.

### EXAMPLE 60

### Synthesis of bzld-[G2]-PGLSA-Acid

1.00 g (1.04 mmol) of of bzld-[G2]-PGLSA-TBDPS was dissolved in 75 mL of THF. Next, 1.25 g (3.96 mmol) of tetrabutylammonium fluoride trihydrate was added to the solution and it was stirred at RT for 1 hour. After one hour the reaction was complete as indicated by TLC. The solution was diluted with 25 mL of H₂O and acidified with 1N HCl to a pH of 3. The product was extracted into CH₂Cl₂, dried over Na₂SO₄, concentrated and dried on the vacuum line. The product was purified by column chromatography (0-5% MeOH in CH₂Cl₂; R_{f}=0.24) for 0.65 g of product (87% yield).

¹H NMR (CDCl₃): δ 2.55-2.77 (m, 12H, —CH₂—CH₂), 4.10-4.17, 4.24-4.31 (m, 12H, —CH₂—CH—CH₂—), 4.74-4.75 (m, 2H, —CH₂—CH—CH₂—), 5.28-5.31 (m, 1H, CH), 5.52-5.54 (m, 2H, CH), 7.33-7.38, 7.47-7.49 (m, 10H, arom. bzld CH) ppm. ¹³C NMR (CDCl₃): δ 28.72, 29.03, 29.38 (succ. —CH₂—), 62.68, 66.56, 69.16 (glycerol, —CH₂—), 101.44 (O—CH—O), 126.23, 128.50, 129.33 (arom. CH), 137.75 (arom. bzld —C—), 172.67, 175.16 (succ. —C(═O)—) ppm. GC-MS: 715.2 m/z (M−H⁻) (theory: 716.2 m/z (M⁺)). Elemental analysis: C, 58.71%; H, 5.82% (theory: C, 58.66%; H, 5.63%). SEC: M_{w}=810, Mₙ=800, PDI=1.01.

### EXAMPLE 61

### Synthesis of HO-[G2]-PGLSA-TBDPS

1.55 g (1.62 mmol) of of bzld-[G2]-PGLSA-TBDPS was dissolved in 40 mL of THF and 1.0 g of 20% Pd(OH)₂/C was added. The solution was then placed in a Parr tube on a hydrogenator and shaken under 50 psi H₂ for 4 hours. The solution was then filtered over wet celite, rotoevaporated, and purified by column chromatography (0-25% acetone in EtOAc) to yield 1.12 g of product (95% yield). R_{f}=0.25 (1:3 acetone:EtOAc). ¹H NMR (CDCl₃): δ 1.07 (s, 9H, t-butyl), 2.25 (b s, 4H, —OH), 2.58-2.82 (m, 12H, —CH₂—CH₂), 3.71-3.74, 4.09-4.26 (m, 12H, —CH₂—CH—CH₂—), 4.87-4.99, 5.24-5.25 (m, 3H, —CH₂—CH—CH₂—), 7.34-7.43, 7.63-7.48 (m, 10H, phenyl CH) ppm. ¹³C NMR (CDCl₃): δ 14.52 (—C—(CH₃)₃), 25.78 (—C—(CH₃)₃), 26.99, 29.30, 30.51, 30.81 (succ. —CH₂—), 62.08, 63.44, 68.17, 70.23 (glycerol, —CH₂—), 125.71, 127.96, 130.35, 135.45 (phenyl), 171.94, 172.40 (succ. —C(═O)—) ppm. GC−MS: 779.5 m/z (MH⁺) (theory: 778.3 m/z (M⁺)). SEC: M_{w}=800, Mₙ=792, PDI=1.01

Acetyl Derivative of HO-[G2]-PGLSA-TBDPS:

Compound HO-[G2]-PGLSA-TBDPS was a hydroscopic oil and repeated attempts to obtain satisfactory EA failed. Thus, we decided to prepare the acetyl analog for elemental analysis. 0.55 g (0.70 mmol) of of HO-[G2]-PGLSA-TBDPS was stirred in 40 mL of CH₂Cl₂ with 0.39 g (1.34 mmol) of DPTS, 0.19 mL (3.36 mmol) of freshly distilled acetic acid, and 0.87 g (4.20 mmol) of DCC. The solution was stirred at RT for 18 hours. The DCU precipitate was filtered and the solution was evaporated. The residue was resuspended in a minimum of CH₂Cl₂, cooled to 10° C. and filtered. The resulting solution was concentrated and further purified by column chromatography (0-5% acetone in CH₂Cl₂) to afford 0.49 g of product (66% yield). R_{f}=0.17 (5% acetone in CH₂Cl₂) ¹H NMR (CDCl₃): δ 1.07 (s, 9H, t-butyl), 2.04 (s, 12H, —CH₃), 2.55-2.83 (m, 12H, —CH₂—CH₂), 4.09-4.32 (m, 12H, —CH₂—CH—CH₂—), 5.20-5.29 (m, 3H, —CH₂—CH—CH₂—), 7.32-7.44, 7.61-7.67 (m, 10H, phenyl CH) ppm. ¹³C NMR (CDCl₃): δ 19.10 (—C—(CH₃)₃), 20.67 (OC—CH₃), 26.82 (—C—(CH₃)₃), 28.60, 28.80, 29.10, 30.59 (succ. —CH₂—), 62.11, 62.31, 69.39 (glycerol, —CH₂—), 127.72, 130.09, 131.67, 135.27 (arom. CH), 170.50, 171.33, 171.61 (—C(═O)—) ppm. FAB-MS: 947.9 m/z (MH⁺) (theory: 947.0 m/z (M⁺)).

Elemental analysis: C, 57.15%; H, 6.26% (theory: C, 57.07%; H, 6.17%). SEC: M_{w}=1075, Mₙ=1041, PDI=1.03.

### EXAMPLE 62

### Synthesis of bzld-[G3]-PGLSA-TBDPS

The bzld-[G3]-PGLSA-TBDPS dendron was synthesized by two methods, first by coupling of a bzld-[G2]-PGLSA-acid dendron to a HO-[G1]-PGLSA-TBDPS dendron convergently, and second by coupling compound to a HO-[G2]-PGLSA-TBDPS dendron (7) divergently.

Convergently: 1.05 g (1.47 mmol) of bzld-[G2]-PGLSA-acid was stirred in 75mL of CH₂Cl₂, and 0.29 g (0.67 mmol) of HO-[G1]-PGLSA-TBDPS, 0.20 g (0.67 mmol) DPTS, and 0.41 g (2.00 mmol) DCC were added. The solution was stirred at RT for 48 hours. The DCU precipitate was filtered off and the solution was evaporated. The product was purified by column chromatography (3:7 hexanes: EtOAc, R_{f}=0.08) with a yield of 0.99 g (82% yield).

Divergently: 0.55 g (0.71 mmol) of a HO-[G2]-PGLSA-TBDPS was stirred in 50 mL of CH₂Cl₂, and 0.42 g (1.41 mmol) of DPTS, 0.871 g (3.11 mmol) of 2(cis-1,3-O-Benzylidene Glycerol)Succinic Acid Monoester, and 0.64 g (3.12 mmol) of DCC were added. The solution was stirred under nitrogen at RT for 18 hours. The DCU precipitate was filtered and the solution was evaporated. The product was purified by column chromatography (3:7 hexanes:EtOAc) to afford 0.71 g of product (54% yield). R_{f}=0.08 (3:7 hexanes:EtOAc). ¹H NMR (CDCl₃): δ 1.08 (s, 9H, t-butyl), 2.54-2.92 (m, 28H, —CH₂—CH₂), 4.08-4.15, 4.22-4.27 (m, 28H, —CH₂—CH—CH₂—), 4.71 (s, 4H, —CH₂—CH—CH₂—), 5.21-5.24 (m, 3H, CH), 5.52 (s, 4H, CH), 7.31-7.42, 7.42-7.49, 7.65-7.67 (m, 30H, arom. bzld and phenyl CH) ppm. ¹³C NMR (CDCl₃): δ 19.31 (—C—(CH₃)₃), 27.04 (—C—(CH₃)₃), 29.35, 30.81 (succ. —CH₂—), 62.49, 66.53, 69.16, 69.47 (glycerol, —CH₂—), 101.33 (O—CH—O), 126.21, 127.94, 128.48, 129.26, 130.32, 135.47 (arom. CH), 138.02 (arom. bzld —C—), 171.90, 172.28 (succ. —C(═O)—) ppm. GC-MS: 1825.6 m/z (M−H⁺) (theory: 1827.9 m/z (M⁺)). HR-FAB: 1825.6124 m/z (M−H⁺) (theory: 1826.6233 m/z (M⁺)). Elemental analysis: C, 60.66%; H, 5.85% (theory: C, 61.11%; H, 5.85%). SEC: M_{w}=1830, Mₙ=1810, PDI=1.01.

### EXAMPLE 63

### Synthesis of bzld-[G31-PGLSA-Acid

2.00 g (1.09 mmol) of bzld-[G3]-PGLSA-TBDPS was dissolved in 125 mL of THF. Next, 1.3 g (4.1 mmol) of tetrabutylammonium fluoride trihydrate was added to the solution. The mixture was stirred at RT for 1 hour. After one hour the reaction was complete as indicated by TLC. The solution was diluted with 25 mL of H₂O and acidified with 1N HCl to a pH of 3. The product was extracted into CH₂Cl₂, dried over Na₂SO₄, rotoevaporated and dried on the vacuum line. The product was purified by column chromatography (0-5% MeOH in CH₂Cl₂) to afford 1.44 g of product (83% yield). R_{f}=0.21 (5% MeOH in CH₂Cl₂). ¹H NMR (CDCl₃): δ 2.58-2.75 (m, 28H, —CH₂—CH₂), 4.11-4.16, 4.19-4.27 (m, 28H, —CH₂—CH—CH₂—), 4.71-4.72 (m, 4H, —CH₂—CH—CH₂—), 5.21-5.28 (m, 3H, CH), 5.52-5.53 (m, 4H, CH), 7.32-7.37, 7.46-7.49 (m, 20H, arom. bzld Ch) ppm. ¹³C NMR (CDCl₃): δ 29.05, 29.36 (succ. —CH₂—), 62.51, 66.58, 69.16 (glycerol, —CH₂—), 101.36 (O—CH—O), 126.21, 128.49, 129.29 (arom. CH), 137.95 (arom. bzld —C—), 171.83, 173.01 (succ. —C(═O)—) ppm. GC-MS: 1587.5 m/z (M−H⁺) (theory: 1588.5 m/z (M⁺)). Elemental analysis: C, 58.02%; H, 5.60% (theory: C, 58.18%; H, 5.58%). SEC: M_{w}=1650, Mₙ=1620, PDI=1.02.

### EXAMPLE 64

### Synthesis of HO-[G3]-PGLSA-TBDPS

0.53 g (0.29 mmol) of bzld-[G3]-PGLSA-TBDPS was dissolved in 50 mL of THF in a Parr tube. 0.4 g of 20% Pd(OH)₂/C was added and the flask was evacuated and filled with 50 psi of H₂. The mixture was shaken for 8 hours, then filtered over wet celite. The filtrate was dried to produce a clear oil which was purified by column chromatography (0-50% acetone in EtOAc) to afford 0.38 g of product (88% yield). R_{f}=0.23 (1:1 acetone:EtOAc). ¹H NMR (CDCl₃): δ 1.3 (s, 9H, t-butyl), 2.52-2.86 (m, 28H, —CH₂—CH₂), 3.44-3.94 (m, 24, —CH₂—CH—CH₂— and —OH), 4.10-4.38, (m, 12H, —CH₂—CH—CH₂—), 4.82-4.92 (m, 4H, CH), 5.18-5.30 (m, 3H, CH), 7.28-7.43, 7.50-7.54, 7.60-7.66 (m, 10H, phenyl CH) ppm. ¹³C NMR (CDCl₃): δ 19.04 (—C—(CH₃)₃), 24.44 (—C—(CH₃)₃), 26.76, 27.12, 28.82, 28.97, 29.10, 30.57 (succ. —CH₂—), 61.17, 62.33, 63.21, 69.30, 75.52 (glycerol, —CH₂—), 127.72, 130.11, 131.57, 134.36, 135.20 (arom. CH), 171.66, 171.72, 171.99, 172.27, 172.38, 172.46 (succ. —C(═O)—) ppm. MALDI-MS: 1475.56 m/z (MH⁺) (theory: 1475.5 m/z (M⁺)). SEC: M_{w}=2101, Mₙ=1994, PDI=1.05.

Acetyl Derivative of Compound of HO-[G3]-PGLSA-TBDPS:

Compound HO-[G3]-PGLSA-TBDPS was a hydroscopic oil and repeated attempts to obtain satisfactory EA failed. Thus, we decided to prepare the acetyl analog for elemental analysis. 0.24 g (0.16 mmol) of HO-[G3]-PGLSA-TBDPS was stirred in 40 mL of CH₂Cl₂ with 0.19 g (0.65 mmol) of DPTS, 0.09 mL (1.55 mmol) of freshly distilled acetic acid, and 0.40 g (1.94 mmol) of DCC. The solution was stirred at RT for 18 hours. The DCU precipitate was filtered and the solution was evaporated. The residue was resuspended in a minimum of CH₂Cl₂, cooled to 10° C. and filtered. The resulting solution was concentrated and further purified by column chromatography (8:2 hexanes:EtOAc to 3:7 hexanes:EtOAc) to afford 0.18 g of product (63% yield). R_{f}=0.15 (3:7 hexanes:EtOAc) ¹H NMR (CDCl₃): δ 1.10 (s, 9H, t-butyl), 1.99 (s, 24H, —CH₃), 2.48-2.78 (m, 28H, —CH₂—CH₂), 4.02-4.30 (m, 28H, —CH₂—CH—CH₂—), 5.12-5.26 (m, 7H, —CH₂—CH—CH₂—), 7.25-7.38, 7.55-7.61 (m, 10H, phenyl CH) ppm. ¹³C NMR (CDCl₃): δ 18.87 (—C—(CH₃)₃), 20.46 (OC—CH₃), 26.61 (—C—(CH₃)₃), 26.95, 28.47, 28.55, 28.64, 28.90, 30.39 (succ. —CH₂—), 61.90, 62.10, 69.02, 69.22 (glycerol, —CH₂—), 127.52, 129.90, 131.48, 135.05 (arom. CH), 170.26, 171.14, 171.40, 171.46 (—C(═O)—) ppm. FAB-MS: 1812.2 m/z (MH⁺) (theory: 1811.8 m/z (M⁺)). Elemental analysis: C, 53.95%; H, 6.12% (theory: C, 53.70%; H, 5.90%). SEC: M_{w}=1943, Mₙ=1882, PDI=1.03.

### EXAMPLE 65

### Synthesis of bzld-[G41-PGLSA-TBDPS

The bzld-[G4]-PGLSA-TBDPS dendron was synthesized by two methods, first by coupling of bzld-[G2]-PGLSA-acid dendron to a HO-[G2]-PGLSA-TBDPS dendron convergently, and secondly by coupling the monoester 2(cis-1,3-O-Benzylidene Glycerol)Succinic Acid Monoester to a HO-[G3]-PGLSA-TBDPS dendron divergently.

Convergently: 0.14 g (0.18 mmol) of HO-[G2]-PGLSA-TBDPS was dissolved in 30 mL of CH₂Cl₂. Next, 0.05 g (0.18 mmol) of DPTS, 0.82 g (1.10 mmol) of bzld-[G2]-PGLSA-acid and 0.22 g (1.10 mmol) of DCC were added. The solution was stirred at RT under nitrogen for 72 hours. The DCU was filtered, the filtrate was concentrated to dryness and the residue was resuspended in a minimum of cold THF. The solution was filtered, concentrated and purified by column chromatography (1:1 hexanes:EtOAc to 1:4 hexanes:EtOAc, R_{f}=0.14) to afford 0.48 g of product (75% yield).

Divergently: 0.38 g (0.26 mmol) of HO-[G3]-PGLSA-TBDPS was dissolved in 50 mL of CH₂Cl₂. Next, 1.00 g (3.57 mmol) of 2(cis-1,3-O-Benzylidene Glycerol)Succinic Acid Monoester, 0.10 g (0.34 mmol) of DPTS, and 0.656 g (3.57 mmol) of DCC were added to the mixture. The solution was stirred for 48 hours under nitrogen at RT. The DCU precipitate was filtered, concentrated and purified by column chromatography (1:1 hexanes:EtOAc to 1:4 hexanes:EtOAc, R_{f}=0.14) to afford 0.572 g of product (60% yield). ¹H NMR (CDCl₃): δ 1.07 (s, 9H, t-butyl), 2.55-2.77 (m, 60H, —CH₂—CH₂), 4.07-4.15, 4.22-4.25 (m, 60H, —CH, —CH—CH₂—), 4.70 (s, 8H, —CH₂—CH—CH₂—), 5.19-5.21 (m, 7H, CH), 5.51 (s, 8H, CH), 7.30-7.40, 7.46-7.48, 7.63-7.65 (m, 50H, arom. bzld and phenyl CH) ppm. ¹³C NMR (CDCl₃): δ 14.40 (—C—(CH₃)₃), 27.03 (—C—(CH₃)₃), 29.02, 29.35 (succ. —CH₂—), 62.47, 66.53, 69.16, 69.49 (glycerol, —CH₂—), 101.31 (O—CH—O), 126.21, 127.94, 128.48, 129.26, 135.47 (arom. CH), 138.03 (arom. bzld —C—), 171.50, 171.90, 172.27 (succ. —C(═O)—) ppm. MALDI-MS: 3574.54 m/z (MH⁺) (theory: 3573.54 m/z (M⁺)). Elemental analysis: C, 59.49%; H, 5.70% (theory: C, 59.19%; H, 5.74%). SEC: M_{w}=3420, Mₙ=3350, PDI=1.02.

### EXAMPLE 66

### Synthesis of [G3]-PGLSA-bzld Dendrimer

0.019 g (0.084 mmol) of [G0]-PGLSA-OH, 12 was dissolved in 50 mL of CH₂Cl₂. Next, 0.64 g (0.40 mmol) of compound bzld-[G3]-PGLSA-acid, 0.074 g (0.25 mmol) of DPTS, and 0.10 g of DCC (0.50 mmol) were added. The solution was stirred for 72 hours at RT under nitrogen. The DCU was filtered off and the filtrate was concentrated. The additional DCU was precipitated in cold THF and filtered. The product was purified by column chromatography (0-5% MeOH in CH₂Cl₂) to yield 0.40 g of product (73% yield). ¹H NMR (CDCl₃): δ 2.60-2.74 (m, 116H, —CH₂—CH₂), 4.08-4.17 (m, 60H, —CH₂—CH—CH₂—), 4.22-4.26 (m, 60H, —CH₂—CH—CH₂—), 4.70 (s, 16H, —CH₂—CH—CH₂—), 5.20-5.23 (m, 14H, CH), 5.51 (s, 16H, CH), 7.32-7.36, 7.46-7.48 (m, 80H, arom. bzld CH) ppm. ¹³C NMR (CDCl₃): δ 29.02, 29.35 (succ. —CH₂—), 62.47, 66.54, 69.16 (glycerol, —CH₂—), 101.31 (O—CH—O), 126.21, 128.48, 129.26 (arom. CH), 138.01 (arom. bzld —C—), 171.83, 172.29 (succ. —C(═O)—) ppm. MALDI: 6553.4 m/z (MH⁺) (theory: 6552.2 m/z (M⁺). Elemental analysis: C, 58.50%; H, 5.48% (theory: C, 58.29%; H, 5.57%). SEC: M_{w}=4740, Mₙ=4590, PDI=1.01.

### EXAMPLE 67

### Synthesis of [G3]-PGLSA-OH Dendrimer, 14

0.33 g (0.051 mmol) of [G3]-PGLSA-bzld was dissolved in 50 mL of a 9:1 solution of THF and MeOH in a Parr tube. Next, 0.50 g of 20% Pd(OH)₂/C was added and the flask was evacuated and filled with 50 psi of H2. The mixture was shaken for 7 hours, then filtered over wet celite. The filtrate was dried to produce 0.25 g of a clear oil (0.049 mmol, 97% yield). ¹H NMR (CD₃OD): _{—}2.64 (m, 116, —CH₂—CH₂—), 3.51 (m, 26, —CH₂—CH—CH₂—), 3.67 (m, 28, —CH₂—CH—CH₂—), 3.80 (m, 12, —CH₂—CH—CH₂—), 4.05 (m, 14, —CH₂—CH—CH₂—), 4.14 (m, 14, —CH₂—CH—CH₂—), 4.22 (m, 22, —CH₂—CH—CH₂—), 4.30 (m, 22, —CH₂—CH—CH₂—), 5.26 (m, 14, —CH₂—CH—CH₂) ppm. ¹³C NMR (CD₃OD): 28.61 (CH₂), 62.41 (CH₂), 62.87 (CH₂), 65.67 CH₂), 67.64 (CH), 69.91 (CH), 172.86 (COOR) ppm. MALDI-MS: 5144.8 rn/z (MH⁺) (theory: 5142.5 m/z (M⁺)). Elemental analysis: C, 48.07%; H, 5.84% (theory: C, 48.11%; H, 5.84%). SEC M_{w}: 5440; Mₙ: 5370; PDI: 1.01.

### EXAMPLE 68

### Synthesis of [G3]-PGLSA-MA Dendrimer (50% Derivatized)

0.22 g (0.041 mmol) of [G3]-PGLSA-OH was dissolved in 5 nL of DMF. Next, 0.20 g (1.66 mmol) of DMAP was then added followed by 0.10 mL (0.67 mmol, 0.5 eq. to the peripheral hydroxyl groups on [G3]-PGLSA-OH) of freshly distilled methacrylic anhydride. After 4.5 hours the reaction was complete as indicated by TLC. 0.03 mL (0.67 mmol) of MeOH was added to the reaction and allowed to stir for an additional 20 minutes. The solution was precipitated into 300 mL of cold ethyl ether. The ether was decanted off and the remaining oily reside was diluted with 20 mL of CH₂Cl₂. The organic phase was washed with 1 N HCl and brine. The organic phase was dried over Na₂SO₄, flitered, and concentrated to approximately 2 mL. This concentrated solution was precipitated in 300 mL of cold ethyl ether. The ether was decanted off and the resulting oily residue was dried under reduced pressure to yield 0.20 g of product (78% yield). ¹H NMR (CDCl₃): δ 1.90 (s, 42H, —CH₃), 2.55-2.77 (m, 116H, —CH₂—CH₂), 3.61-3.78 (m, 30H, —CH₂—CH—CH₂—), 4.07-4.30 (m, 120H, —CH₂—CH—CH₂—), 5.58-5.62 (m, 16H, ═CH), 6.03-6.16 (m, 16H, ═CH) ppm. ¹³C NMR (CDCl₃): δ 18.24 (—CH3), 29.56, 29.75 (succ. —CH₂—), 61.52, 62.09, 62.14, 65.17, 65.83, 69.39, 69.56, 70.04, 73.23, 75.89 (glycerol —CH₂—), 171.04, 171.25, 171.37, 171.58, 171.79, 172.14, 172.51 ppm. MALDI-MS: 6224.6 m/z (MH⁺) (theory: 6231.6 m/z (M⁺)). SEC: M_{w}=3525, Mₙ=2708, PDI=1.30.

### EXAMPLE 69

### Synthesis of bzld-[G3]-PGLSA-PEG-OMe

0.29 g (0.18 mmol) of bzld-[G3]-PGLSA-acid was dissolved in 75 mL of CH₂Cl₂. Next 0.45 g (0.09 mmol) of 5000 MW poly(ethylene glycol) mono-methyl ether (PEG-OMe; MALDI-MS: M_{w}=5147, Mₙ=5074, PDI=1.01), 0.037 g (0.18 mmol) of DCC, and 0.026 g (0.09 mmol) of DPTS were added to the solution. The solution was stirred under nitrogen at RT for 168 hours. The DCU was filtered and the filtrate was concentrated to dryness. The resulting residue was resuspended in THF, cooled, and the DCU was filtered. The resulting solution was precipitated in ethyl ether. The solid was dissolved in THF, stirred with Amberlyst A-21 ion-exchange resin (Aldrich) (weakly basic resin) to eliminate the excess 9. The solution was filtered and the filtrate was dried over Na₂SO₄, dissolved in CH₂Cl₂, washed with 0.1 N HCl, and dried over Na₂SO₄ to yield 0.53 g of a solid white product (89% yield). ¹H NMR (CDCl₃): δ 2.60-2.73 (m, 28H, —CH₂—CH₂), 3.36 (s, MME CH₃) 3.57-3.64 (m, 406H, PEG CH₂), 4.11-4.26 (m, 28H, —CH₂—CH—CH₂—), 4.71 (m, 4H, —CH₂—CH—CH₂—), 5.21-5.23 (m, 3H, Ch), 5.52-5.54 (m, 4H, CH), 7.32-7.37, 7.46-7.49 (m, 20H, arom. bzld CH) ppm. ¹³C NMR (CDCl₃): δ 29.36, 29.90 (succ. —CH₂—), 62.48, 66.53, 69.17 (glycerol, —CH₂—), 70.77 (PEG, —CH₂—), 101.33 (O—CH—O), 126.21, 128.48, 129.26 (arom. CH), 137.80 (arom. bzld —C—), 171.90 (succ. —C(═O)—) ppm. MALDI-MS: M_{w}=6671, Mₙ=6628 PDI=1.01 (theoretical MW=6588). SEC: M_{w}=6990, Mₙ=6670, PDI=1.04.

### EXAMPLE 70

### Synthesis of HO-[G3]-PGLSA-PEG-OMe

0.52 g of bzld-[G3]-PGLSA-PEG-OMe was dissolved in 40 mL of THF. Next, 0.10 g of 20% Pd(OH)₂/C was added. The reaction vessel was evacuated and flushed with hydrogen. The solution was shaken for 3 hours under 50 psi H₂ at RT. The Pd(OH)₂/C was removed by filtering over wet celite. The filtrate was dried and precipitated in ethyl ether to yield 0.40 g of an opaque hydroscopic solid (83% yield). ¹H NMR (CDCl₃): δ 2.60-2.70 (m, 28H, —CH₂—CH₂), 3.36 (s, MME CH₃) 3.53-3.78 (b m, 422H, PEG CH₂ and —CH, —CH—CH₂—), 4.17-4.27 (m, 12H, —CH₂—CH—CH₂—), 4.92 (m, 4H, —CH₂—CH—CH₂—), 5.21-5.23 (m, 3H, CH) ppm. ¹³C NMR (DMSO): δ 29.14, 29.36 (succ. —CH₂—), 60.25 (—CH₃ OMe), 63.22, 66.54, 69.87 (glycerol, —CH₂—), 70.43 (PEG, —CH₂—), 172.35, 172.57 (succ. —C(═O)—) ppm. MALDI-MS: M_{w}=6302, Mₙ=6260, PDI=1.01 (theoretical MW=6136). SEC: M_{w}=6660, Mₙ=6460, PDI=1.03.

### EXAMPLE 71

### Synthesis of MA-[G3]-PGLSA-PEG-OMe

0.39 g (0.064 mmol) of HO-[G3]-PGLSA-PEG-OMe was dissolved in 30 mL of CH₂Cl₂. Next, 10 mg (0.08 mmol) of DMAP and 0.15 mL methacrylic anhydride (1.0 mmol) were added and the solution was stirred at RT under nitrogen overnight. The solution was then washed with 0.1 N HCl, dried over Na₂SO₄, condensed, and precipitated in ether to afford 0.41 g of product (96% yield). ¹H NMR (CDCl₃): δ 1.92 (s, 24H, —CH₃— methacrylate), 2.63 (m, 28H, —CH₂—CH₂), 3.36 (s, MME CH₃) 3.59-3.67 (m, 406H, PEG CH₂), 4.19-4.39 (m, 28H, —CH₂—CH—CH₂—), 5.24 (m, 4H, —CH₂—CH—CH₂), 5.35 (m, 3H, CH), 5.59 (s, 8H, —CH₂— methacrylate), 6.10 (s, 8H, —CH₂— methacrylate) ppm. MALDI-MS: M_{w}=7080, Mₙ=7008, PDI=1.01 (theoretical MW=6780). SEC: M_{w}=6918, Mₙ=6465, PDI=1.07.

### EXAMPLE 72

### Synthesis of Myr-[G2]-PGLSA-TBDPS

0.45 g (0.58 mmol) of compound OH-[G2]-PGLSA-TBDPS was dissolved in 75 mL of CH₂Cl₂ with 0.63 g (2.77 mmol) of myristic acid (Myr), 0.34 g (1.16 mmol) of DPTS, and 0.72 g (3.47 mmol) of DCC. The reaction was stirred at RT for 16 hours. The DCU precipitate was filtered and the solution was evaporated. The residue was resuspended in 50 mL of ethanol, cooled to 0° C. for 6 hours and filtered. The precipitate was resuspended in 75 mL of CH₂Cl₂, washed with 75 mL of H₂O, dried over Na₂SO₄, and the solvent evaporated to yield 0.84 g of product (89% yield). ¹H NMR (CDCl₃): δ 0.80-0.89 (t, 12H, —CH₃), 1.08 (s, 9H, t-butyl), 1.14-1.34 (m, 80H, myristic —CH₂—), 1.50-1.64 (m, 8H, C(═O)—CH₂—CH₂—CH₂—), 2.22-2.33 (t, 8H, C(═O)—CH₂—CH₂—), 2.53-2.83 (m, 12H, succinic —CH₂—CH₂), 4.08-4.34 (m, 12H, —CH₂—CH—CH₂—), 5.18-5.30 (m, 3H, —CH₂—CH—CH₂—), 7.32-7.44, 7.61-7.67 (m, 10H, phenyl CH) ppm. ¹³C NMR (CDCl₃): δ 14.25, 22.67, 24.81, 26.85, 28.81, 28.79, 29.12, 29.24, 29.36, 29.53, 29.64, 31.97, 34.05, 61.88, 62.34, 69.17, 127.66, 130.13, 135.28, 138.77, 171.34, 171.69, 173.32 ppm. FAB-MS: 1620.1 m/z (MH⁺) (theory: 1620.29 m/z (M⁺)). Elemental analysis: C, 68.84%; H, 9.69% (theory: C, 68.94%; H, 9.58%). SEC: M_{w}=2168, Mₙ=2135, PDI=1.02.

### EXAMPLE 73

### Synthesis of Myr-[G2]-PGLSA-Acid

0.81 g (0.50 mmol) of Myr-[G2]-PGLSA-TBPDS was dissolved in 100 mL of THF. Next, 0.55 g (1.75 mmol) of tetrabutylammonium fluoride trihydrate was added to the solution. The mixture was stirred at RT for 1 hour. After one hour the reaction was complete as indicated by TLC. The solution was diluted with 25 mL of H₂O and acidified with 1N HCl to a pH of 3. The product was extracted into EtOAc, dried over Na₂SO₄, rotoevaporated and dried on the vacuum line. The product was purified by column chromatography (0-3% MeOH in CH₂Cl₂) to afford 0.60 g of product (87% yield). R_{f}=0.23 (3% MeOH in CH₂Cl₂). ¹H NMR (CDCl₃): δ 0.82-0.88 (t, 12H, —CH₃), 1.20-1.31 (m, 80H, myristic —CH₂—), 1.53-1.64 (m, 8H, —C(═O)—CH₂—CH₂—CH₂—), 2.26-2.33 (t, 8H, —C(═O)—CH₂—CH₂—), 2.60-2.68 (m, 12H, —CH₂—CH₂—), 4.11-4.34 (m, 12H, —CH₂—CH—CH₂—), 5.19-5.35 (m, 3H, —CH₂—CH—CH₂—) ppm. ¹³C NMR (CDCl₃): δ 14.16, 22.78, 24.98, 28.56, 28.87, 29.07, 29.24, 29.47, 29.63, 29.87, 32.01, 34.04, 62.02, 62.64, 69.16, 69.93, 171.47, 171.68, 173.51 ppm. FAB-MS: 1382.9 m/z (M−H⁺) (theory: 1381.9 m/z (M⁺)). Elemental analysis: C, 66.72%; H, 9.91% (theory: C, 66.92%; H, 9.92%). SEC: M_{w}=2074, Mₙ=2040, PDI=1.02.

### EXAMPLE 74

### Synthesis of 2-benzyl-1,3-di(Myr-[G2]-PGLSA)₂-glycerol

0.85 g (0.62 mmol) of compound Myr-[G2]-PGLSA-acid was dissolved in 75 mL of CH₂Cl₂ with 0.05 g (0.26 mmol) of 2-benzyl-glycerol, 0.08 g (0.26 mmol) of DPTS, and 0.16 g (0.77 mmol) of DCC. The reaction was stirred at RT for 16 hours. The DCU precipitate was filtered and the solution was evaporated. The residue was resuspended in 50 mL of ethanol, cooled to 0° C. for 6 hours and filtered. The precipitate was purified by column chromatography (20-50% EtOAc in hexanes) to yield 0.63 g of product (85% yield). R_{f}=0.17 (30% EtOAc in hexanes). ¹H NMR (CDCl₃): δ 0.81-0.88 (t, 24H, —CH₃), 1.17-1.34 (m, 160H, myristic —CH₂—), 1.52-1.63 (m, 16H, C(═O)—CH₂—CH₂—CH₂—), 2.24-2.32 (t, 16H, C(═O)—CH₂—CH₂—), 2.58-2.66 (m, 24H, succinic —CH₂—CH₂), 3.77-3.85 (m, 1H, —CH₂—CH—CH₂—), 4.04-4.38 (m, 28H, —CH₂—CH—CH₂—), 4.59-4.65 (s, 2H, benzyl —CH₂—), 5.17-5.34 (m, 6H, —CH₂—CH—CH₂—), 7.25-7.34 (m, 5H, aromatic CH) ppm. ¹³C NMR (CDCl₃): MALDI-MS: 2933.4 m/z (M+Na⁺) (theory: 2933.0 m/z (M+Na⁺)).

Elemental analysis: C, 67.92%; H, 9.79% (theory: C, 67.69%; H, 9.77%). SEC: M_{w}=4388, Mₙ=4258, PDI=1.03.

### EXAMPLE 75

### Synthesis of 1,3-di(Myr-[G2]-PGLSA)₂-glycerol

0.47 g (0.16 mmol) of 2-benzyl-1,3-di(Myr-[G2]-PGLSA)₂-glycerol was dissolved in 20 mL of THF and 0.5 g of 10% Pd/C was added. The solution was then placed in a Parr tube on a hydrogenator and shaken under 50 psi H₂ for 10 hours. The solution was then filtered over wet celite, rotoevaporated, to yield the product.

### EXAMPLE 76

### Synthesis of bz-SA-[G2]-PGLSA-TBDPS

0.77 g (0.99 mmol) of compound HO-[G2]-PGLSA-TBDPS was dissolved in 75 mL of CH₂Cl₂ with 0.99 g (4.76 mmol) of benzylated succinic acid (bz-sa), 0.58 g (1.98 mmol) of DPTS, and 1.23 g (5.91 mmol) of DCC. The reaction was stirred at RT for 16 hours. The DCU precipitate was filtered and the solution was evaporated. The residue was resuspended in a minimum of CH₂Cl₂, cooled to 10° C. for 1 hour and filtered. The solution was concentrated under reduced pressure and purified by column chromatorgraphy (30-50% EtOAc in hexanes) to afford 1.21 g of product (79% yield). R_{f} 0.18 (40% EtOAc in hexanes). ¹H NMR (CDCl₃): δ 1.08 (s, 9H, t-butyl), 2.55-2.81 (m, 28H, succinic —CH₂—CH₂), 4.06-4.37 (m, 12H, —CH₂—CH—CH₂—), 5.11 (s, 8H, benzyl —CH₂—), 5.18-5.29 (m, 3H, —CH₂—CH—CH₂—), 7.22-7.44, 7.61-7.67 (m, 30H, aromatic CH) ppm. ³C NMR (CDCl₃): δ 19.13, 26.81, 28.42, 28.64, 28.70, 28.91, 29.07, 30.56, 62.68, 66.72, 69.07, 73.69, 127.68, 128.23, 128.54, 130.06, 131.73, 135.21, 135.77, 171.64, 171.73, 171.90 ppm. FAB-MS: 1539.6 m/z (MH⁺) (theory: 1539.7 m/z (M⁺)). Elemental analysis: C, 63.35%; H, 6.02% (theory: C, 63.19%; H, 5.89%).

### EXAMPLE 77

### Synthesis of bz-SA-[G2]-PGLSA-Acid

1.12 g (0.73 mmol) of bz-SA-[G2]-PGLSA-TBDPS was dissolved in 100 mL of THF. Next, 0.89 g (2.76 mmol) of tetrabutylammonium fluoride trihydrate was added to the solution. The mixture was stirred at RT for 1 hour. After one hour the reaction was complete as indicated by TLC. The solution was diluted with 25 mL of H₂O and acidified with 1N HCl to a pH of 3. The product was extracted into EtOAc, dried over Na₂SO₄, rotoevaporated and dried on the vacuum line. The product was purified by column chromatography (0-3% MeOH in CH₂Cl₂) to afford 0.71 g of product (75% yield). R_{f}=0.18 (3% MeOH in CH₂Cl₂). ¹H NMR (CDCl₃): δ 2.54-2.69 (m, 28H, —CH₂—CH₂), 4.11-4.31 (m, 12H, —CH₂—CH—CH₂—), 5.09 (s, 8H, benzyl —CH₂—), 5.18-5.25 (m, 3H, —CH₂—CH—CH₂—), 7.25-7.36 (m, 20H, aromatic CH) ppm. ¹³C NMR (CDCl₃): δ 28.57, 28.78, 28.94, 62.28, 62.43, 66.60, 69.16, 69.37, 128.24, 128.29, 128.61, 128.57, 171.33, 171.79, 171.95 ppm. FAB-MS: 1301.5 m/z (M−H⁺) (theory: 1301.3 m/z (M⁺)).

Elemental analysis: C, 60.23%; H, 5.81% (theory: C, 60.00%; H, 5.58%). SEC: M_{w}=1415, Mₙ=1379, PDI=1.03.

### EXAMPLE 78

### Synthesis of bz-SA-[G4]-PGLSA-TBDPS

0.07 g (0.08 mmol) of compound HO-[G2]-PGLSA-TBDPS was dissolved in 40 mL of CH₂Cl₂ with 0.53 g (0.41 mmol) of bz-SA-[G2]-PGLSA-acid, 0.05 g (0.17 mmol) of DPTS, and 0.11 g (0.51 mmol) of DCC. The reaction was stirred at RT for 48 hours. The DCU precipitate was filtered and the solution was evaporated. The residue was resuspended in a minimum of CH₂Cl₂, cooled to 10° C. for 1 hour and filtered. The solution was concentrated under reduced pressure and purified by column chromatorgraphy (30-80% EtOAc in hexanes) to afford 0.40 g of product (80% yield). R_{f}=0.18 (65% EtOAc in hexanes). ¹H NMR (CDCl₃): δ 1.07 (s, 9H, t-butyl), 2.53-2.81 (m, 124H, succinic —CH₂—CH₂), 4.10-4.31 (m, 60H, —CH₂—CH—CH₂—), 5.09 (s, 32H, benzyl —CH₂—), 5.18-5.28 (m, 15H, —CH₂—CH—CH₂—), 7.25-7.41, 7.45-7.49, 7.61-7.66 (m, 90H, aromatic CH) ppm. ¹³C NMR (CDCl₃): δ 26.72, 28.52, 28.73, 28.87, 62.15, 66.43, 68.84, 69.16, 125.91, 127.64, 128.11, 128.33, 128.46, 130.01, 135.16, 135.66, 171.25, 171.54, 171.64, 171.81 ppm. MALDI-MS: XXX m/z (MH⁺) (theory: XXX m/z (M⁺)).

Elemental analysis: C, 60.70%; H, 5.74% (theory: C, 60.34%; H, 5.63%). SEC: M_{w}=5142, Mₙ=5064, PDI=1.02.

### EXAMPLE 79

### Synthesis of bz-SA-[G4]-PGLSA-Acid

0.22 g (0.04 mmol) of bz-SA-[G4]-PGLSA-TBDPS was dissolved in 12 mL of THF. Next, 0.04 g (0.13 mmol) of tetrabutylammonium fluoride trihydrate was added to the solution. The mixture was stirred at RT for 4 hours. The solution was diluted with 5 mL of H₂O and acidified with 1N HCl to a pH of 3. Additional THF was added dropwise to keep product in solution. The product was extracted into EtOAc, dried over Na₂SO₄, rotoevaporated and dried on the vacuum line. The product was purified by column chromatography (20-100% EtOAc in hexanes) to afford the product (XX% yield). R_{f}=XX (XX% EtOAc in hexanses). ¹H NMR (CDCl₃): δ 2.46-2.84 (m, 124H, —CH₂—CH₂), 4.12-4.49 (m, 60H, —CH₂—CH—CH₂—), 5.02-5.36 (m, 57H, benzyl —CH₂— and —CH₂—CH—CH₂—), 7.25-7.48 (m, 80H, aromatic CH) ppm. ¹³C NMR (CDCl₃): δ 28.79, 28.93, 62.21, 66.51,69.24, 127.64, 128.17, 128.52, 135.69, 171.34, 171.73, 171.91 ppm.

### EXAMPLE 80

### Synthesis of Lys3 Dendron

DCC (5.45 g, 26 mmol) was added in five portions over 10 minutes to a solution of ZLys(Z)OH (10 g, 24 mmol) and 1.1 equiv of pentafluorophenol in freshly distilled CH₂Cl₂ (40 ml). The reaction mixture was stirred under N₂ at 25° C. for 2 h, filtered to remove the insoluble urea, concentrated to ˜20 ml under reduced pressure, and then stored at 4° C. for 2 h. An additional filtration removed further urea, and the filtrate was diluted with hexane (25 ml) and stored at 4° C. for 4 h. The resultant white precipitate was collected by filtration, washed with DCM/hexane (1:2, 3×5 ml), and dried in vacuum; yield 13.37 g (98%).

Synthesis of ZLys(Z)Lys(ZLys(Z))OMe

LysOMe. 2HCl (1.43 g, 6 mmol) was dissolved in DMF (45 ml) with the DIEA (2.35 g, 18 mmol), and then the HOBT (2.25 g, 14 mmol) was added. After 5 minutes ZLys(Z)OPFP (12.5 g, 21 mmol) in DCM (30 ml) was added at 0° C. for 10 minutes. The mixture was stirred for 24 h at RT under N₂After concentration under vacuum the mixture was dissolved in DCM (50 ml) washed with NaHCO₃ (2×150 ml), water (2×150 ml) and then dried over NaSO₄. The solvent was removed, and the mixture was precipitated in ether to lead a pure white compound 5.72 g (98%).

Synthesis of LysLys(Lys)OMe. 4HCl

Pd/C (10% w/w) was added to a solution of ZLys(Z)Lys(ZLys(Z))OMe (1 g, 1 mmol) in MeOH (50 ml). The flask for catalytic hydrogenolysis was evacuated and filled with 50 psi of H₂ before shaking for 10 h. The catalyst was filtered and washed with MeOH (20 ml). The filtered was acidified with HCl gas. The acid solution was evaporated to give 578 mg of the white compound (98%).

### EXAMPLE 81

### Synthesis of Lys3Cys4 Dendron

Synthesis of IsoCys(Boc)OPFP

Real numbers DCC (4.11 g, 20 mmol) was added in five portions over 10 min to a solution of IsoCys(Boc)OH (4.8 g, 18 mmol) and 1.1 equiv of pentafluorophenol (3.42, 20 mmol)in freshly distilled CH₂Cl₂ (25 ml). The reaction mixture was stirred under N₂ at 25° C. for 2 h, filtered to remove the insoluble urea, concentrated to ˜20 ml under reduced pressure, and then stored at 4° C. for 2 h. An additional filtration removed further urea, and the product was crystallized from hot hexane. The resultant white precipitate was collected by filtration and dried in vacuum; yield 5.8 g (95%).

Synthesis of isoCys(Boc)Lys(isoCys(Boc))Lys(isoCys(Boc)Lys(isoCys(Boc))) OMe

LysLys(Lys)OMe.4HCl, (500 mg, 0.8 mmol) was dissolved in DMF (25 ml) with DIEA (550 mg, 4 mmol, and then HOBT (695 mg, 4 mmol) was added. After 5 minutes the IsoCys(Boc)OPFP, (2.78 g, 5.6 mmol) in DCM (21 ml) was added at 0° C. for 10 minutes. The mixture was stirred for 24 h at RT under N₂. After concentration under vacuum the mixture was dissolved in DCM (40 ml) washed by NaHCO₃ (2×100 ml), water (2×100 ml) and dried over NaSO₄. Evaporation of organic solvent gave an oil that was purified by silica gel chromatography (DCM-MeOH=96/4): yield 951 mg (74%).

Synthesis of isoCysLys(isoCys)Lys(isoCysLys(isoCys))OMe

TFA (5 ml) was added in 10 portions over 10 min to a solution of isoCys(Boc)Lys(isoCys(Boc))Lys(isoCys(Boc)Lys(isoCys(Boc))) OMe, (600 mg, 0.4 mmol) in freshly distilled CH₂Cl₂ (30 ml) at 0° C. The reaction mixture was stirred under N₂ for 25° C. for 2 h. The solvent was removed by vacuum, and the mixture was precipitated in ether to afford a pure white compound 417 mg (97%).

Synthesis of CysLys(Cys)Lys(CysLys(Cys))OMe

isoCysLys(isoCys)Lys(isoCysLys(isoCys))OMe, (400 mg, 0.4 mmol) was dissolved in HCl 1N-MeOH 50/50 (60 ml), and stirred under N₂ at 25° C. for 4 h. The solvent was removed by vacuum, and the mixture was precipitated in ether to lead a pure white compound 350 mg (90%).

### EXAMPLE 82

### Synthesis of the Dimethyl Acetal Succinic Ester)₂—PEG

(succinic acid)₂—PEG

(OH)₂—PEG (10 g, 3 mmol) was dissolved in pyridine (30 ml) with succinic anhydride (5.88 g, 60 mmol), and stirred under N₂ at 25° C. for 4 h. The solvent was removed by vacuum, and the mixture was precipitated in ether to afford a product 10.48 g (99%)

(succinic acid cesium salt)₂—PEG

(succinic acid)₂—PEG (1 g, 0.3 mmol) was dissolved in water and the pH was adjusted to 7.5 with CSCO₃. The solvent was removed to obtain the pure compound (99%).

(dimethyl acetal succinic ester)₂—PEG

(dimethyl acetal succinic ester)₂—PEG was prepared in by reacting of (succinic acid cesium salt)₂—PEG, (1 g, 0.3 mmol), with bromoacetaldehyde dimethyl acetal (133 μl, 1.2 mmol) in DMF (5 ml) at 60° C. for 3 days. The solvent was removed by vacuum, and the mixture was precipitated in ether.

(dialdehyde succinic ester)₂—PEG

(dialdehyde succinic ester)₂—PEG was obtain by treatment of (dimethyl acetal succinic ester)₂—PEG, with TFA (5% H₂O) in CH₂Cl₂ (1:3) at room temperature for 20 minutes. The solvent was removed by vacuum, and the product was precipitated in ethyl ether.

### EXAMPLE 83

### Preparation of a Covalently Crosslinked Gel/Network

The gel was prepared by mixing an aqueous solution of the lys3Cys4 dendrons with the peg-dialdehyde. For example, the dendron dissolved at 33% w/w in buffer HEPES pH=7 (10 mg dendron in 20 μl) and the PEG dialdehyde (commercially available, Mw=3400) was dissolved at 55% w/w (50 mg PEG dialdehyde in 40 μl) in the same buffer. These two solutions were mixed together to lead a gel. Gelation occurs almost immediately.

### EXAMPLE 84

### Preparation of a Non-Covalently Crosslinked Gel/Network

(didodecane methyl amine)₂—PEG

The (didodecane methyl amine)₂—PEG was prepared in two steps by first treating (NH₂)—PEG with 8 equivalents of bromododecane, 15 equivalents of NaCO₃ in reflux ethanol to obtain (didodecane amine)₂—PEG. The (didodecane amine)₂—PEG, 1, was then treated with methyl iodine to afford (didodecane methyl amine)₂—PEG after precipitation in ether.

This cationic-hydrophobic linear polymer is likely to form a gel with the carboxylated terminated dendritic polymers.

### EXAMPLE 85

General Procedure for the Eye Surgeries. An enucleated human eye (NC Eye Bank) was placed under a surgical microscope with the cornea facing upwards. The corneal epithelium was scraped with a 4.1 mm keratome blade, and then a 2.75 mm keratome blade was used to incise the central cornea. Next the keratome blade was used to form the 4.1 mm linear laceration. The wound was closed with either 3 interrupted 10-0 nylon sutures or the self-gelling crosslinkable biodendritic copolymer. Next, a 25 gauge butterfly needle connected to a syringe pump (kdscientific, Model 100 series) was inserted into the scleral wall adjacent to an ocular muscle. In order to measure the wound leaking pressures, the eye was connected to a cardiac transducer via a 20 gauge needle which was inserted 1 cm through the optic nerve. The needle was held in place with surgical tape. The pressure was then recorded. The syringe pump dispensed buffered saline solution (at a rate of 15-20 mL/hr) into the eye while the pressure was simultaneously read on the cardiac transducer. The syringe pump rate was maintained to achieve a continuous 1 mm Hg increase in pressure. The leak pressure was recorded as the pressure at which fluid was observed to leak from the eye under the surgical microscope.

An enucleated eye with the cornea facing upwards was held under a surgical microscope and a 4.1 mm laceration was made with a keratome blade. This wound was then closed using either three interrupted 10-0 nylon sutures in a standard 3-1-1 suturing configuration or the crosslinkable biodendritic copolymer. The crosslinkable polymer system contained the lys3Cys4 dendron and PEG-dialdehyde (3400 mw). The crosslinkable polymer system was then applied to the wound and it sealed the wound in less than 20 seconds. Next, saline was injected in the anterior chamber via a syringe inserted through the scleral wall adjacent to an ocular muscle until the repaired laceration leaked. A cardiac transducer probe inserted approximately 1 cm through the optic nerve monitored the leaking pressure for both the nylon suture (N=6) and biodendrimer sealant (N=4) treated eyes. For reference, normal intraocular pressure in a human eye is between 15 and 20 mm Hg. The mean leaking pressures (LP) for the sutured treated eyes was 90 mm Hg. The mean leaking pressures (LP) for the polymer treated eyes was approximately the same.

### EXAMPLE 86

### General Procedure for Securing a LASIK Flap

LASIK (laser-assisted in situ keratomileusis) is the popular refractive surgical procedure where a thin, hinged corneal flap is created by a microkeratome blade. This flap is then moved aside to allow an excimer laser beam to ablate the corneal stromal tissue with extreme precision for the correction of myopia (near-sightedness) and astigmatism. At the conclusion of the procedure, the flap is then repositioned and allowed to heal. However, with trauma, this flap can become dislocated prior to healing, resulting in flap striae (folds) and severe visual loss. When this complication occurs, treatment involves prompt replacement of the flap and flap suturing. The use of sutures has limitations and drawbacks as discussed above. For the LASIK flap study, hinged corneal flaps were created using the Hansatome microkeratome system on four human donor eyebank eyes. Flap adherence was tested with dry Merocel sponges and tying forceps. Biodendrimer tissue adhesive was applied to the entire flap edge and then polymerized with an argon laser beam. The biodendrimer sealant successfully sealed the flap.

### EXAMPLE 87

### General Procedure for the Preparation of an Endocapsular Lens

The gel mixture was prepared directly by mixing together both solutions of dendrone and PEG dialdehyde. The measurement was measured after a 20 min waiting period. The measured refractive index for the gel at 25° was 1.41 and at 37° C. was 1.39. The natural lens has a refractive index between 1.399 and 1.425.

### EXAMPLE 88

### Method of Encapsulation of a Drug in a Dendritic Polymer

A generation four (G4) poly(glycerol-succinic acid) dendrimer was synthesized in a divergent manner by successive coupling (esterification) and deprotection (hydrogenolysis) reactions with 2-(cis-1,3-O-benzylidene-glycerol)succinic acid mono ester and H₂/Pd(OH)₂, respectively. A carboxylate terminated G4 dendrimer, ([G4]-PGLSA-COONa) was also prepared by reacting the [G4]-PGLSA-OH dendrimer with succinic anhydride in pyridine. The hydroxyl (OH) and carboxylated (CO₂H) terminated dendrimers with molecular weights of 10700 and 18500 amu, respectively, were characterized by NMR, MALDI mass spectrometry, SEC, and quasi-elastic light scattering.

The encapsulation procedure requires both the dendrimer and hydrophobic compound/pharmaceutical to be soluble in a volatile organic solvent that is miscible with water. The following is a typical procedure for the encapsulation of a hydrophobic moiety. First a 1:1 molar ratio of the dendrimer to encapsulant is dissolved in 1.5-2.0 mL of methanol, and agitated for 10 minutes. Water (1.0 mL) is then added to the solution and stirred for one hour at ambient temperature. Finally, the methanol is removed over several hours via rotary evaporation.

### EXAMPLE 89

### Encapsulation of a 10-hydroxycamptothecin in a Dendritic Polymer

10-hydroxycamptothecin (10HCPT) was encapsulated in the dendritic polymer as described above. This poorly water-soluble anticancer drug (˜6 μM) was encapsulated in the [G4]-PGLSA-COONa dendrimer at a concentration of 200 μM. Initial attempts with the hydroxy terminated [G4]-PGLSA-OH dendrimer were less successful. The aromatic and vinyl protons of the encapsulated 10HCPT are clearly visible and distinct from the dendrimer protons in the ¹H NMR spectrum (spectrum not shown). The fluorescence spectrum in water of 10HCPT in a [G4]-PGLSA-COONa dendrimer shows a _max at 434 nm (excitation 370 nm).

### EXAMPLE 90

### Characterization of a Poorly Water Soluble Drug in a Dendrimer. Encapsulation of Reichardt's dye in a Dendritic Polymer

Characterization data on the dendrimer/encapsulant supramolecular complex is highly desirable. Consequently, we have performed a series of NMR experiments with a model drug “Reichardt's dye” since this poorly water soluble drug (10⁻⁶ M) possess a large number of aromatic protons. This increases the likelihood for success and allows us to develop the techniques prior to investigating the encapsulated camptothecin. We propose to a) probe the molecular interactions in the G4 dendrimer/encapsulant complexes using NMR techniques.

We performed a series of 1D and 2D NMR experiments to gain insight into the nature of the encapsulatant-dendrimer complex. Reichardt's dye was selected as the encapsulant model for these experiments since it possesses a large number of aromatic hydrogens. The 1D ¹H NMR spectrum in D₂O of the [G4]-PGLSA-OH encapsulated dye shows substantial line broading of the aromatic protons compared to unencapsulated Reichardt's dye in CD₃OD. The =5 fold increase in line broadening (FWHM) is attributed to the restricted tumbling of the encapsulated dye. The singlet resonances from the pyridino and phenolato 3,5 protons of the dye in CD₃OD resonate at 8.40 and 6.73 ppm, respectively. When encapsulated in the [G4]-PGLSA-OH dendrimer in D₂O, these signals shift downfield to 8.52 and 7.04 ppm, respectively. ¹H NMR spin-lattice relaxation time constants (T₁) of these two signals decreased from 1.5 and 1.8 s in CD₃OD to 0.90 and 0.89 s respectively, when encapsulated in the [G4]-PGLSA-OH dendrimer in D₂O. Similarly, upon encapsulation, the succinic acid methylenes of the [G4]-PGLSA-OH shift upfield from 2.7 to 2.6 ppm as a consequence of the ring current effects associated with the aromatic rings of Reichardt's dye.

Next, ¹H 2D NOESY NMR spectra were recorded to explore the molecular interactions between the dendrimer and the encapsulated Reichardt's dye. The NOESY spectra were collected at 25° C. with a mixing time of 450 ms, and NOE between the dye and the dendrimer are clearly observed. The relatively long mixing time was used to provide time for buildup of intermolecular NOEs (which are governed by the specific intermolecular dipole-dipole T₁ relaxation times). The longer mixing times did not change the NOEs. We will conduct experiments with shorter mixing times in the near future. Not only does Reichardt's dye show a number of intramolecular NOE cross peaks among its aromatic protons, but a large number of intermolecular NOE cross peaks are also observed between the aromatic protons of Reichardt's dye and the methylenes of succinic acid and the methines and methylenes of glycerol of the dendrimer demonstrating significant close range NOE dipolar interactions. The extensive network of NOEs raises concerns regarding spin diffusion; however, the differing T₁ relaxation times of the dendrimer and the encapsulant suggest that the cross peaks arise from distinct NOE interactions. Since the intramolecular distance between the pyridino and phenolato 3,5 protons of Reichardt's dye is about 3 Å, we estimate the intermolecular cross peaks to indicate distances of 5 Å or less between the dye and the dendrimer.

Furthermore, when the 2D NOESY diagonal is phased negative, the off-diagonal NOE cross peaks from the dendrimer and dye also phased negatively. This indicates that all of the NOEs are associated with motions typical of a large macromolecule, further confirming that the dye is encapsulated within the dendrimer. In contrast, when a 2D ¹H NMR NOESY spectrum was obtained for the Reichardt's dye in CD₃OD and the diagonal peaks are phased negative, all of the off-diagonal cross peaks are positive, consistent with NOEs of small molecules. These data demonstrate that 1) the dye is tumbling on the same time scale as the dendrimer, and 2) the association between the dye and dendrimer is sufficiently strong to observe significant dipolar through space NOE effects.

### EXAMPLE 91

### Cytotoxicity of Encapsulated 10-hydroxycamptothecin in a Dendritic Polymer

We evaluated the anticancer activity of the encapsulated 10HCPT using a standard NCI assay. Varying concentrations of [G4]-PGLSA-COONa encapsulated 10HCPT were incubated for 0.5 to 2 hours with MCF-7 human breast cancer cells. No cytotoxic effects were observed with the biodendrimer, whereas cell viability was significantly reduced upon incubation with the encapsulated 10HCPT. The highest concentration of encapsulated 10HCPT (20 μM) showed significant activity with less then 10% of the cells remaining viable. These in vitro results demonstrate that the anticancer activity of 10HCPT is retained after encapsulation within the biodendrimer and that the biodendrimer itself is a suitable delivery vehicle for hydrophobic anticancer drugs.

Varying concentrations of [G4]-PGLSA-COONa encapsulated 10HCPT were also incubated for 0.5 to 2 hours with colon cancer cells. Similar results were observed with no cytotoxic effects with the biodendrimer, whereas cell viability was significantly reduced upon incubation with the encapsulated 10HCPT.

We claim:

1. Dendritic polymers or copolymers composed of building blocks that are biocompatible or are natural metabolites in vivo including but not limited to glycerol, lactic acid; glycolic acid, glycerol, amino acids, caproic acid, ribose, glucose, succinic acid, malic acid, amino acids, peptides, synthetic peptide analogs, poly(ethylene glycol), poly(hydroxyacids) [e.g., PGA. PLA].

2. A dendritic polymer or monomer according to claim 1 for medical use.

3. A dendritic polymer or monomer according to claim 1 for wound care or wound management.

4. A dendritic polymer or monomer according to claim 1 as a tissue sealant.

5. A dendritic polymer or monomer according to claim 1 for reconstructive, cosmetic, or plastic surgery.

6. A dendritic polymer or monomer according to claim 1 for seeding cells in vitro for subsequent in vivo placement.

7. A dendritic polymer or monomer according to claim 1 for prevention of adhesion.

8. A dendritic polymer or monomer according to claim 1 for organ repair or restoration.

9. A dendritic polymer or monomer according to claim 1 for delivery of therapeutics.

10. A dendritic polymer or monomer according to claim 1 for drug delivery.

11. A dendritic polymer or monomer according to claim 1 for gene delivery.

11. A dendritic polymer or monomer according to claim 1 for medical imaging.

12. A crosslinkable/polymerizable dendritic polymer or monomer according to claim 1 for wound care or wound management.

13. A crosslinkable/polymerizable dendritic polymer or monomer according to claim 1 as a tissue sealant.

14. A crosslinkable/polymerizable dendritic polymer or monomer according to claim 1 as a lens.

15. A crosslinkable/polymerizable dendritic polymer or monomer according to claim 1 for seeding cells in vitro for subsequent in vivo placement.

16. A crosslinkable/polymerizable dendritic polymer or monomer according to claim 1 for seeding with cells and subsequent in situ polymerization in vivo.

17. A crosslinkable/polymerizable dendritic polymer or monomer according to claim 1 for prevention of adhesion.

18. A crosslinkable/polymerizable dendritic polymer or monomer according to claim 1 for organ repair or restoration.

19. A crosslinkable/polymerizable dendritic polymer or monomer according to claim 1 for delivery of therapeutics.

20. A crosslinkable dendritic or dendritic polymer according to claim 1 for drug delivery.

21. A crosslinkable dendritic or dendritic polymer polymer according to claim 1 for gene delivery.

22. A crosslinkable dendritic or dendritic polymer polymer according to claim 1 for medical imaging.

23. A crosslinkable dendritic or dendritic polymer polymer according to claim 1 for reconstructive, cosmetic or plastic surgery

24. A crosslinkable dendritic polymer or monomer according to claim 1 wherein the crosslinking is of covalent, ionic, electrostatic, and/or hydrophobic nature.

25. A crosslinkable dendritic polymer or monomer according to claim 1 wherein the crosslinking reaction involves a nucleophile and electrophile.

26. A crosslinkable dendritic polymer or monomer according to claim 1 wherein the crosslinking reaction is a peptide ligation reaction.

27. A crosslinkable dendritic polymer or monomer according to claim 1 wherein the crosslinking reaction is a Diels-Alder reaction.

28. A crosslinkable dendritic polymer or monomer according to claim 1 wherein the crosslinking reaction is a Michale Addition reaction.

29. A crosslinkable dendritic polymer or monomer according to claim 1 wherein the crosslinking reaction is a photochemical reaction using a UV or vis photoinitator chromophore.

30. A crosslinkable dendritic or dendritic polymer according to claim 1 in combination with a linear, comb, multi-block, star polymer(s) for a medical or tissue engineering application.

31. A crosslinkable dendritic or dendritic polymer according to claim 1 in combination with a crosslinkable linear, comb, multi-block, star polymer(s) for a medical or tissue engineering application.

32. A crosslinkable dendritic or dendritic polymer according to claim 1 in combinaton with a crosslinkable monomer(s) for a medical or tissue engineering application.

33. A crosslinkable dendritic or dendritic polymer according to claim 1 is combined with a crosslinkable small molecule(s) (molecule weight less than 1000 daltons) for a medical or tissue engineering application.

34. A crosslinkable dendritic or dendritic polymer or monomer according to claim 1 wherein the said crosslinking dendritic polymer is combined with one or more linear, comb, multi-block, star polymers or crosslinkable comb, multi-block, star polymers.

35. A crosslinkable dendritic polymer or monomer according to claim 1 wherein the final polymeric form is a gel, film, fiber, or woven sheet.

36. A dendritic polymer or monomer according to claim 1 wherein the final polymeric form is a gel, film, fiber, or woven sheet.

37. A dendritic polymer or monomer according to claim 1 wherein the dendritic structure is asymmetric at the surface such as a surface block structure where a carboxylate acid(s) and alkyl chains, or acrylate(s) and PEG(s) are present, for example, or within the core and inner layers of the dendrimer such as amide and ester linkages in the structure.

38. A crosslinkable or noncrosslinkable polymer according to claim 1 wherein the polymer is a star biodendritic polymer or copolymer as shown in at least one of the formulas below: where 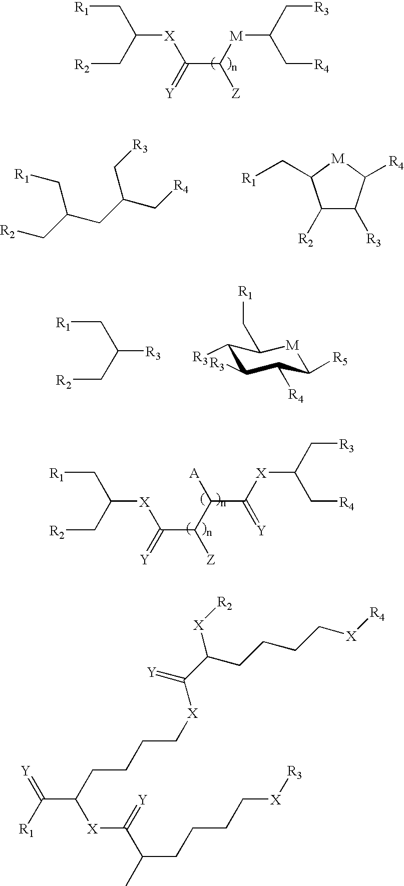
Y and X are the same or different at each occurrence and are O, S, Se, N(H), or P(H) and where R₁, R₂, R₃, R₄, R₅, A or Z are the same or different and include —H, —CH₃, —OH, carboxylic acid, sulfate, phosphate, aldehyde, methoxy, amine, amide, thiol, disulfide, straight or branched chain alkane, straight or branched chain alkene, straight or branched chain ester, straight or branched chain ether, straight or branched chain silane, straight or branched chain urethane, straight or branched chain, carbonate, straight or branched chain sulfate, straight or branched chain phosphate, straight or branched chain thiol urethane, straight or branched chain amine, straight or branched chain thiol urea, straight or branched chain thiol ether, straight or branched chain thiol ester, or any combination thereof.

39. A crosslinkable or noncrosslinkable polymer according to claim 38 where the straight or branched chain is of 1-50 carbon atoms wherein the chain is fully saturated, fully unsaturated or any combination therein

40. A crosslinkable or noncrosslinkable polymer according to claim 38 where the straight or branched chain is of 1-50 carbon atoms wherein the chain is fully saturated, fully unsaturated or any combination therein.

41. A crosslinkable or noncrosslinkable polymer according to claim 38 wherein straight or branched chains are the same number of carbons or different wherein R₁, R₂, R₃, R₄, R₅, A or Z are any combination of the linkers including ester, silane, urea, amide, amine, urethane, thiol-urethane, carbonate, thio-ether, thio-ester, sulfate, phosphate and ether.

42. A crosslinkable or noncrosslinkable polymer according to claim 38 which includes at least one chain selected from the group consisting of hydrocarbons, flourocarbons, halocarbons, alkenes, and alkynes.

43. A crosslinkable or noncrosslinkable polymer according to claim 38 which includes at least one chain selected from the group consisting of linear and dendritic polymers.

44. A crosslinkable or noncrosslinkable polymer according wherein said wherein said linear and dendritic polymers include at least one selected from the group consisting of polyethers, polyesters, polyamines, polyacrylic acids, polycarbonates, polyamino acids, polynucleic acids and polysaccharides of molecular weight ranging from 200-1,000,000, and wherein said chain contains 0, 1 or more than 1 photopolymerizable group.

45. A crosslinkable or noncrosslinkable polymer, wherein the polyether is PEG, and wherein the polyester is PLA, PGA or PLGA.

46. A polymer of claim 40 or a linear polymer wherein the chain is a polymer or copolymer of a polyester, polyamide, polyether, or polycarbonate of or the polymer in claim 40 in combination with a polyester, polyamide, polyether, or polycarbonate of:

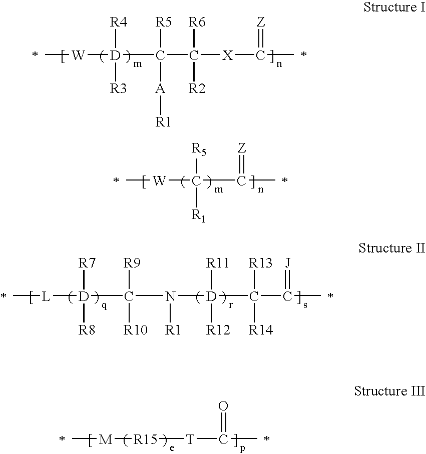

47. A polymer of claim 46 comprised of repeating units of general Structure I, where A is O, S, Se, or N-R7.

48. A polymer as in claim 46, where W, X, and Z are the same or different at each occurrence and are O, S, Se, N(H), or P(H).

49. A polymer as in claim 46, where R1 is hydrogen, a straight or branched alkyl chain of 1-20 carbons, cycloalkyl, aryl, olefin, silyl, alkylsilyl, arylsilyl, alkylaryl, or arylalkyl group.

50. A polymer as in claim 46, where R1 is hydrogen, a straight or branched alkyl chain of 1-20 carbons, cycloalkyl, aryl, olefin, silyl, alkylsilyl, arylsilyl, alkylaryl, or arylalkyl group substituted internally or terminally by one or more hydroxyl, hydroxyether, carboxyl, carboxyester, carboxyamide, amino, mono- or di-substituted amino, thiol, thioester, sulfate, phosphate, phosphonate, or halogen substituents.

51. A polymer as in claim 46, where R1 is a polymer (such as poly(ethylene glycol), poly(ethylene oxide), or a poly(hydroxyacid)), a carbohydrate, a protein, a polypeptide, an amino acid, a nucleic acid, a nucleotide, a polynucleotide, any DNA or RNA segment, a lipid, a polysaccharide, an antibody, a pharmaceutical agent, or any epitope for a biological receptor.

52. A polymer as in claim 46, where R1 is a photocrosslinkable, chemically, or ionically crosslinkable group.

53. A polymer as in any one of claims 46-51, in which D is a straight or branched alkyl chain of 1-5 carbons, m is 0 or 1, and R2, R3, R4, R5, R5, and R7 are the same or different at each occurrence and are hydrogen, a straight or branched alkyl chain of 1-20 carbons, cycloalkyl, aryl, alkoxy, aryloxy, olefin, alkylamine, dialkylamine, arylamine, diarylamine, alkylamide, dialkylamide, arylamide, diarylamide, alkylaryl, or arylalkyl group.

54. A polymer of claim 48 comprised of repeating units of General Structure II, where L, N, and J are the same or different at each occurrence and are O, S, Se, N(H), or P(H).

55. A polymer as in claim 48 where R1 is hydrogen, a straight or branched alkyl chain of 1-20 carbons, cycloalkyl, aryl, olefin, silyl, alkylsilyl, arylsilyl, alkylaryl, or arylalkyl group.

56. A polymer as in claim 48 where R1 is hydrogen, a straight or branched alkyl chain of 1-20 carbons, cycloalkyl, aryl, olefin, silyl, alkylsilyl, arylsilyl, alkylaryl, or arylalkyl group substituted internally or terminally by one or more hydroxyl, hydroxyether, carboxyl, carboxyester, carboxyamide, amino, mono- or di-substituted amino, thiol, thioester, sulfate, phosphate, phosphonate, or halogen substituents.

57. A polymer as in claim 48 where R1 is a polymer selected from the group consisting of poly(ethylene glycols), poly(ethylene oxides), and poly(hydroxyacids, or is a carbohydrate, a protein, a polypeptide, an amino acid, a nucleic acid, a nucleotide, a polynucleotide, a DNA or RNA segment, a lipid, a polysaccharide, an antibody, a pharmaceutical agent, or an epitope for a biological receptor.

58. A polymer as in claim 48 where R1 is a photocrosslinkable, chemically, or ionically crosslinkable group.

59. A polymer as in any one of claims 48-58, where D is a straight or branched alkyl chain of 1-5 carbons, q and r are the same or different at each occurrence and are 0 or 1, and R7, R8, R9, R10, R11, R12, R13, and R14 are the same or different at each occurrence and are hydrogen, a straight or branched alkyl chain of 1-20 carbons, cycloalkyl, aryl, alkoxy, aryloxy, olefin, alkylamine, dialkylamine, arylamine, diarylamine, alkylamide, dialkylamide, arylamide, diarylamide, alkylaryl, or arylalkyl group.

60. A block or random copolymer as in claim 48 comprised of repeating units of general Structure III, where M, T, and Q are the same or different at each occurrence and are O, S, Se, N(H), or P(H), e is 0 or 1-9, and R15 is a straight or branched alkyl chain of 1-5 carbons, unsubstituted or substituted with one or more hydroxyl, hydroxyether, carboxyl, carboxyester, carboxyamide, amino, mono- or di-substituted amino, thiol, thioester, sulfate, phosphate, phosphonate, or halogen substituents

61. A block or random copolymer as in claim 48 comprised of repeating units of general Structure III, where M, T, and Q are the same or different at each occurrence and are O, S, Se, N(H), or P(H), and R15 is a straight or branched alkyl chain of 1-5 carbons, unsubstituted or substituted with one or more hydroxyl, hydroxyether, carboxyl, carboxyester, carboxyamide, amino, mono- or di-substituted amino, thiol, thioester, sulfate, phosphate, phosphonate, or halogen substituents.

62. A block or random copolymer as in claim 48 comprised of repeating units of general Structure III, where M, T, and Q are the same or different at each occurrence and are O, S, Se, N(H), or P(H), and R15 is a straight or branched alkyl chain of 1-5 carbons, unsubstituted or substituted with one or more hydroxyl, hydroxyether, carboxyl, carboxyester, carboxyamide, amino, mono- or di-substituted amino, thiol, thioester, sulfate, phosphate, phosphonate, or halogen substituents.

63. A higher order block or random copolymer comprised of three or more different repeating units, and having one or more repeating units as in any one of claims 48-62.

64. A block or random copolymer as in claim 48, which includes at least one terminal crosslinkable group selected from the group consisting of amines, thiols, amides, phosphates, sulphates, hydroxides, alkenes, and alkynes.

65. A block or random copolymer as in claim 48 where X, Y, M is O, S, N—H, N-R, and wherein R is —H, CH₂, CR₂, Se or an isoelectronic species of oxygen.

66. A block or random copolymer as in claim 48 wherein an amino acid(s) is attached to R₁, R₂, R₃, R₄, R₅, A, and/or Z.

67. A block or random copolymer as in claim 48 wherein a polypeptide(s) is attached to R₁, R₂, R₃, R₄, R₅, A, and/or Z.

68. A block or random copolymer as in claim 48 wherein an antibody(ies) is attached to R₁, R₂, R₃, R₄, R₅, A, and/or Z.

69. A block or random copolymer as in claim 48 wherein a nucleotide(s) is attached to R₁, R₂, R₃, R₄, R₅ A, and/or Z.

70. A block or random copolymer as in claim 48 wherein a nucleoside(s) is attached to R₁, R₂, R₃, R₄, R₅, A, and/or Z.

71. A block or random copolymer as in claim 48 wherein an oligonucleotide(s) is attached to R₁, R₂, R₃, R₄, R₅, A, and/or Z.

72. A block or random copolymer as in claim 48 wherein a ligand(s) is attached to R₁, R₂, R₃, R₄, R₅, A, and/or Z that binds to a biological receptor.

73. A block or random copolymer as in claim 48 wherein a pharmaceutical agent(s) is attached to R₁, R₂, R₃, R₄, R₅, A, and/or Z.

74. A crosslinkable or noncrosslinkable polymer or copolymer according to claim 1 wherein the polymer is a dendritic macromolcule including at least one polymer selected from the group consisting of dendrimers, hybrid linear-dendrimers, dendrons, or hyperbranched polymers according to one of the general formulas or such similar structures below: where R₃, R₄, which may be the same or different, are a repeat pattern of B, and n is 0 to 50.

75. The polymer of claim 74, wherein X, Y, M is O, S, N—H, N—R, wherin R is —H, CH₂, CR₂ or a chain as defined above, Se or any isoelectronic species of oxygen

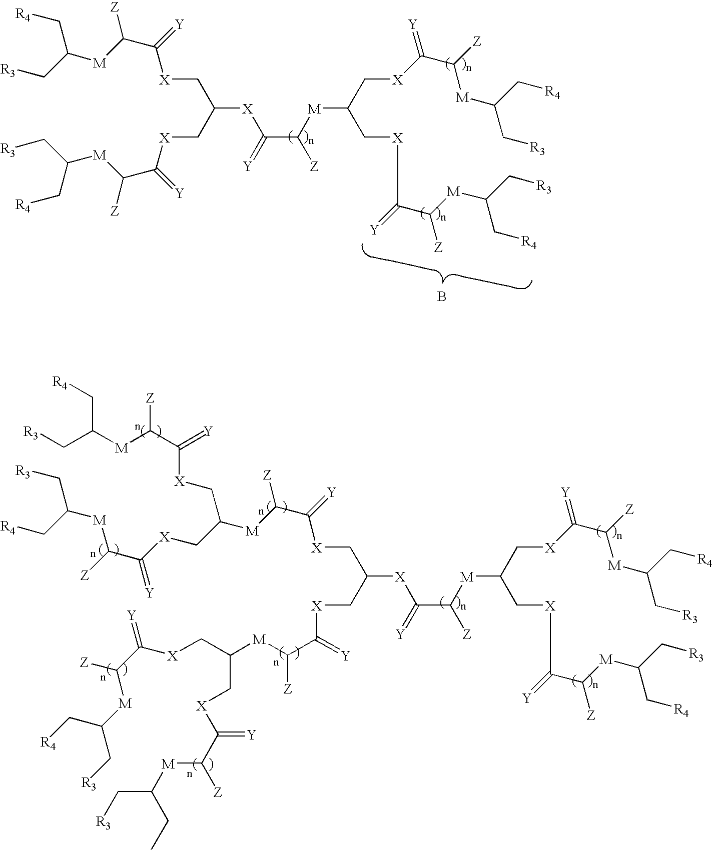

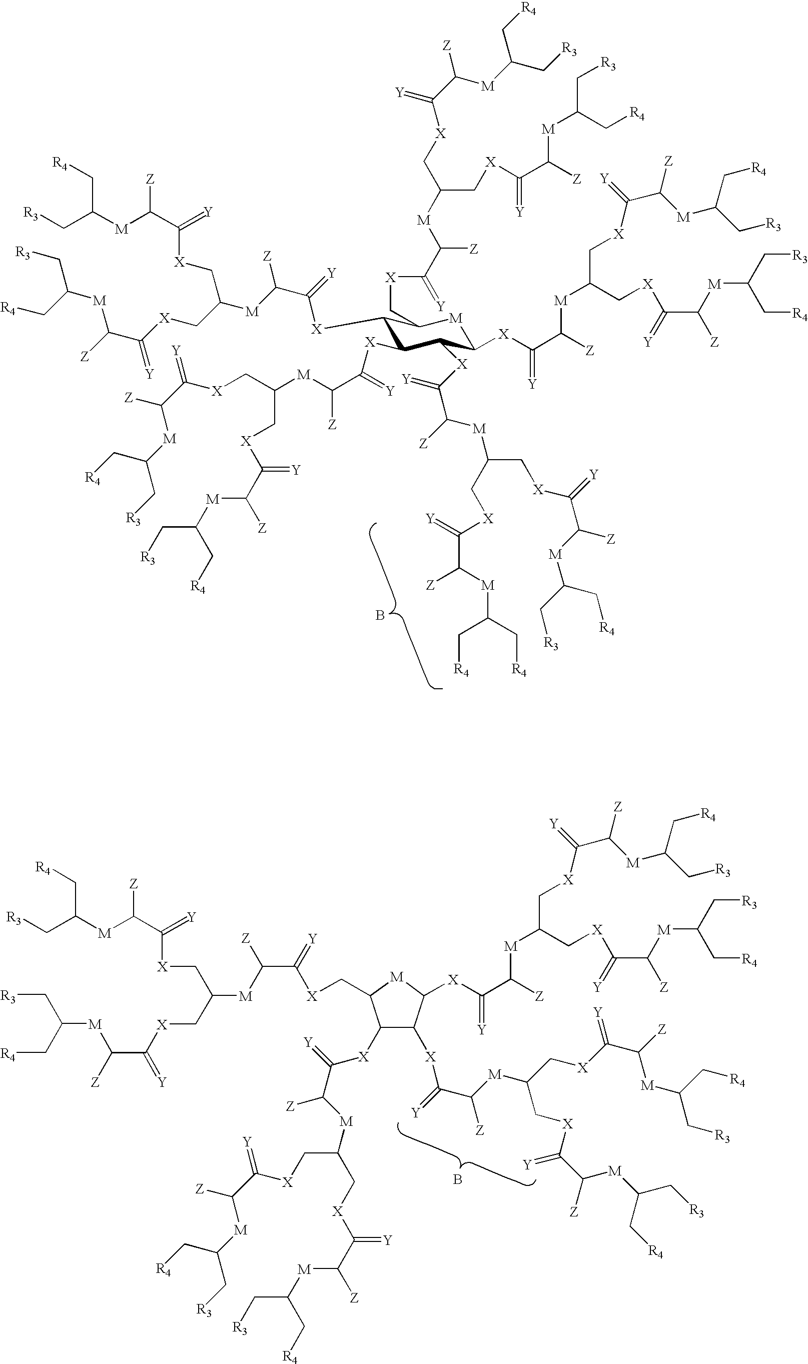

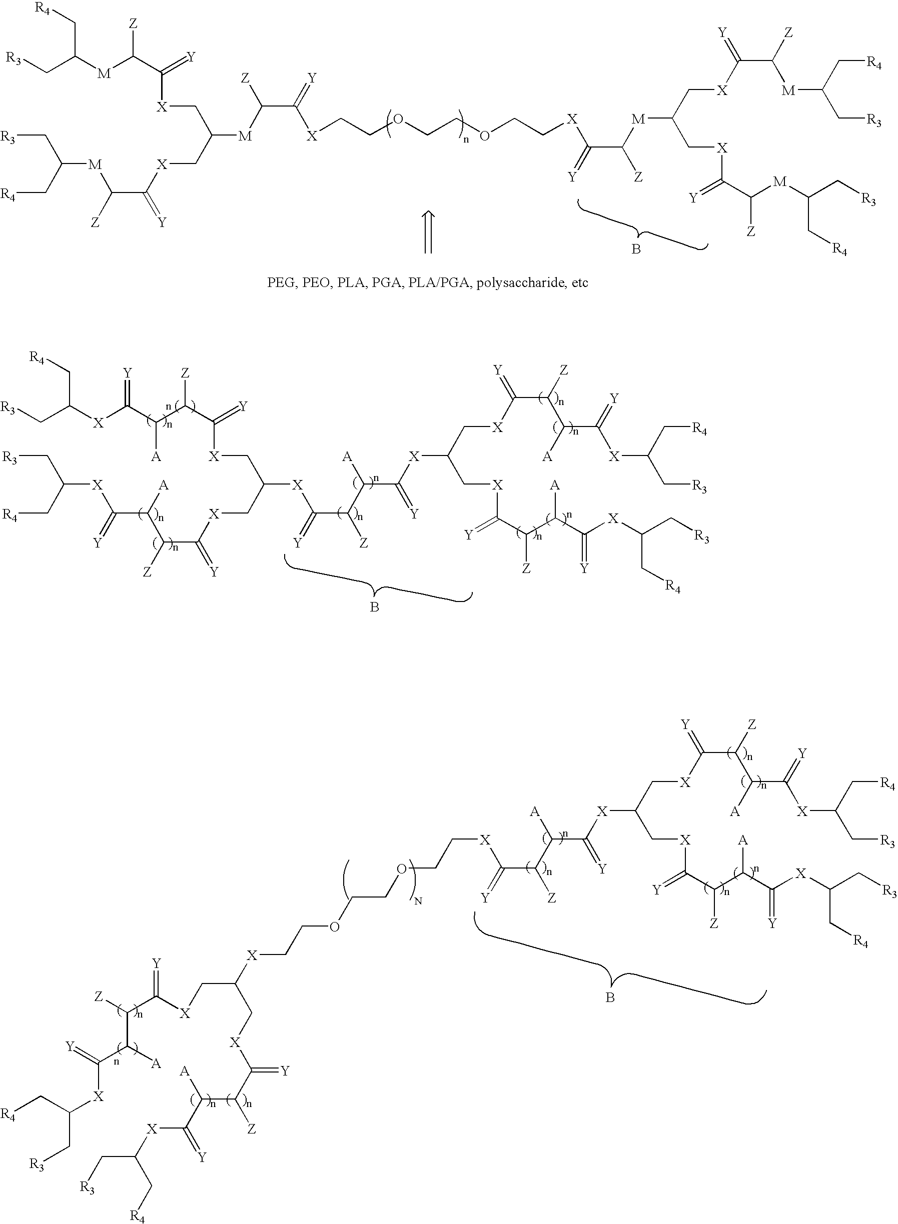

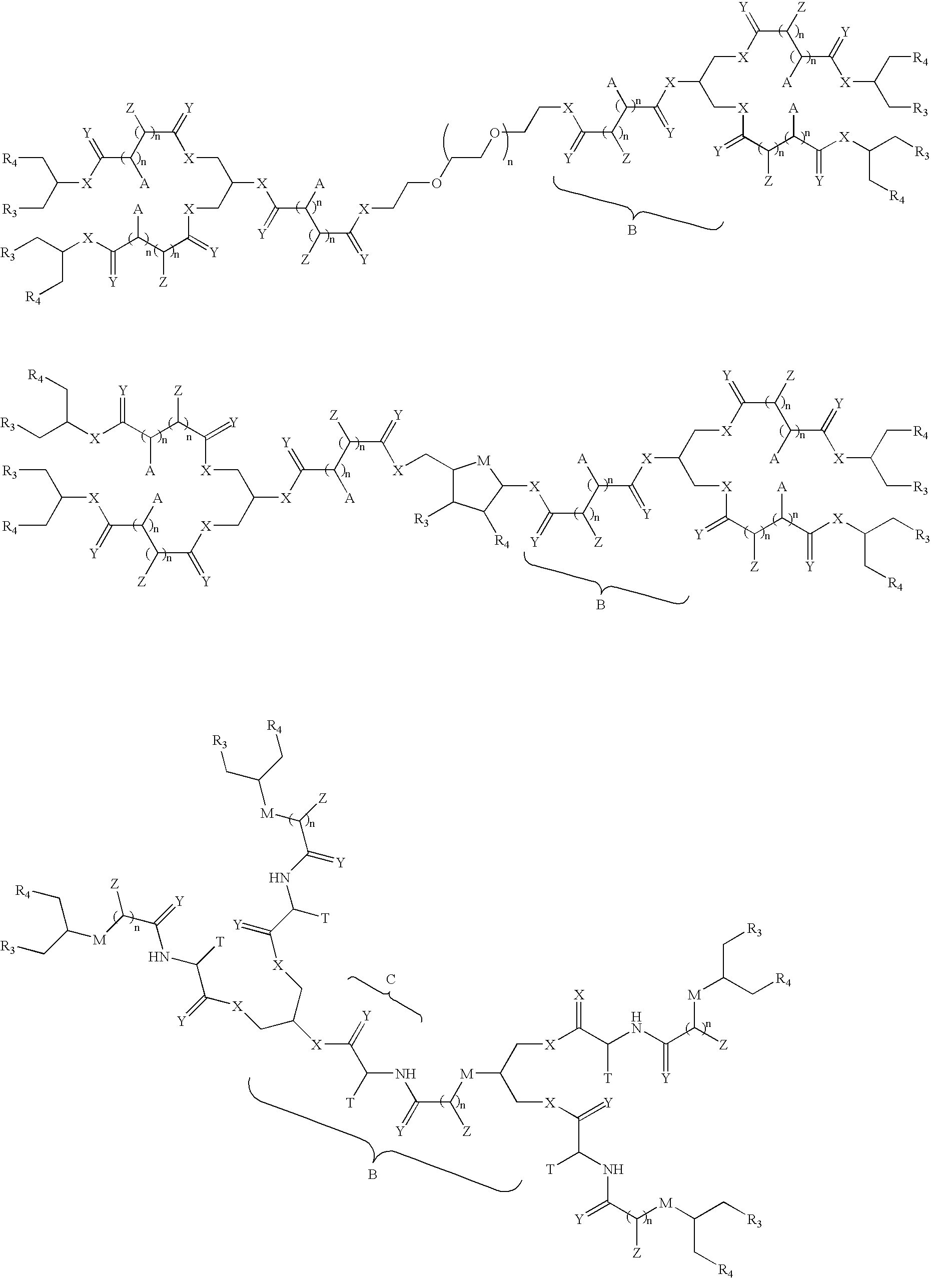

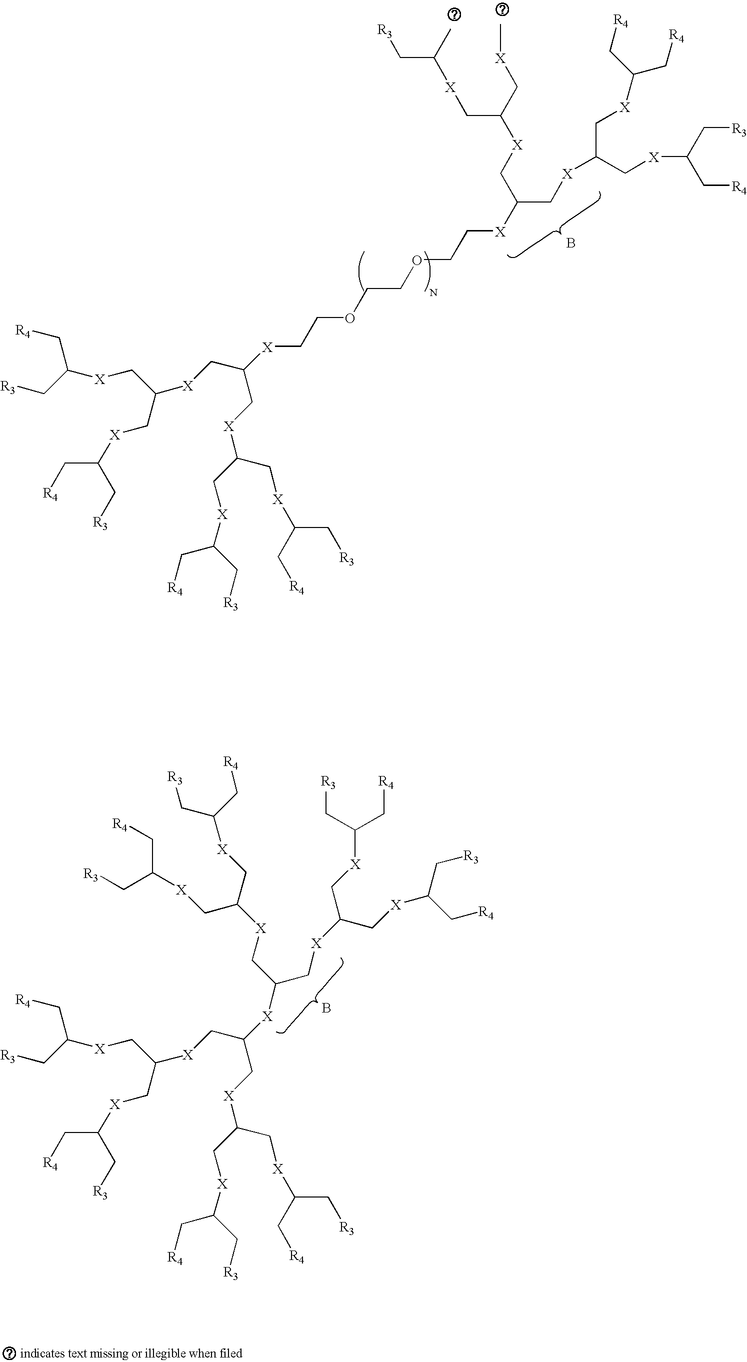

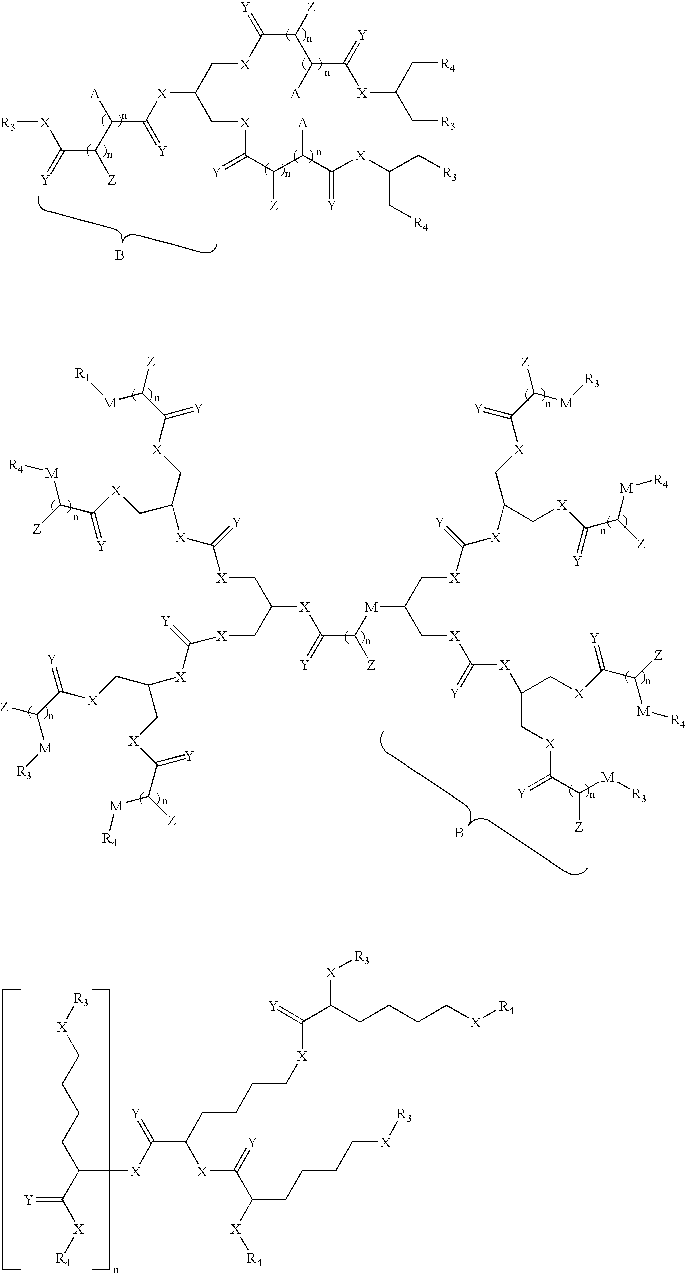

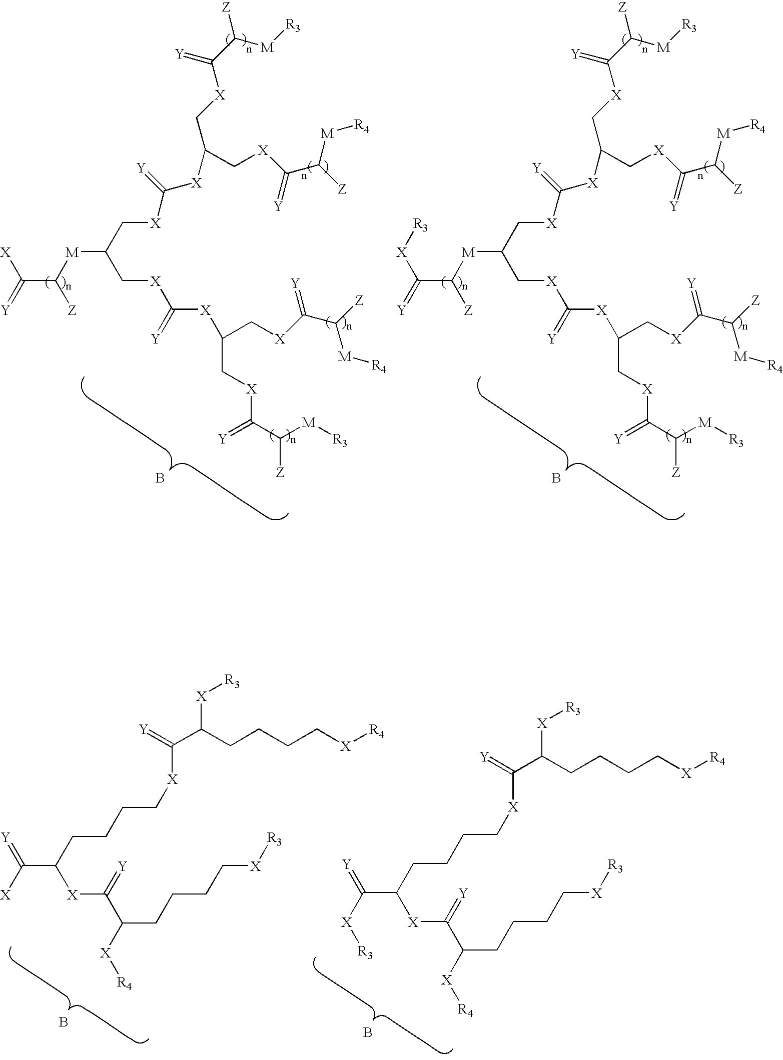

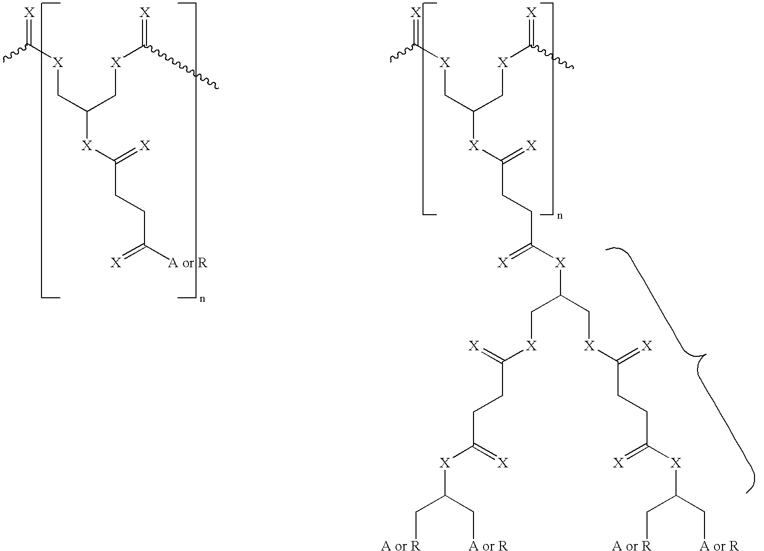

76. The polymer of claim 74, wherein X, Y, M is O, S, N—H, N—R, wherin R is —H, CH₂, CR₂ or a chain as defined above, Se or any isoelectronic species of oxygen.

77. The polymer of claim 74, where R₃ is a carboxycyclic acid protecting group such as but not limtied to a phthalimidomethyl ester, a t-butyldimethylsilyl ester, or a t-butyldiphenylsilyl ester.

78. The polymer of claim 74, where R₃, R₄, A, and Z are the same or different and are —H, —OH, —CH₃, carboxylic acid, sulfate, phosphate, aldehyde, methoxy, amine, amide, thiol, disulfide, straight or branched chain alkane, straight or branched chain alkene, straight or branched chain ester, straight or branched chain ether, straight or branched chain silane, straight or branched chain urethane, straight or branched chain, carbonate, straight or branched chain sulfate, straight or branched chain phosphate, straight or branched chain thiol urethane, straight or branched chain amine, straight or branched chain thiol urea, straight or branched chain thiol ether, straight or branched chain thiol ester, or any combination thereof, and wherein c is a natural or un-natural amino acid.

79. The polymer of claim 74 having a straight or branched chain of 1-50 carbon atoms and wherein the chain is fully saturated, fully unsaturated or any combination therein.

80. The polymer of claim 74 wherein straight or branched chains are the same number of carbons or different and wherein R₃, R₄, A, Z are any combination of linkers selected from the group consisting of esters, silanes, ureas, amides, amines, urethanes, thio]-urethanes, carbonates, carbamates, thio-ethers, thio-esters, sulfates, phosphates and ethers.

81. The polymer of claim 74 wherein chains include at least one selected from hydrocarbons, flourocarbons, halocarbons, alkenes, and alkynes.

82. The polymer of claim 74 wherein said chains include polyethers, polyesters, polyamines, polyacrylic acids, polyamino acids, polynucleic acids and polysaccharides of molecular weight ranging from 200-1,000,000, and wherein said chain contains 1 or more crosslinkable or photopolymerizable group.

83. The polymer of claim 74, wherein the chains include at least one of PEG, PLA, PGA, PGLA, and PMMA.

84. A block or random copolymer as in claim 82, which includes at least one terminal crosslinkable or photopolymerizable group selected from the group consisting of amines, thiols, amides, phosphates, sulphates, hydroxides, alkenes, and alkynes.

85. The polymer of claim 74, wherein an amino acid(s) is attached to Z, A, R₃, and/or R₄.

86. The polymer of claim 74, wherein a polypeptide(s) is attached to Z, A, R₃, and/or R₄.

87. The polymer of claim 74, wherein an antibody(ies) or single chain antibody(ies) is attached to Z, A, R₃, and/or R₄.

88. The polymer of claim 74, wherein a nucleotide(s) is attached to Z, A, R₃, and/or R₄.

89. The polymer of claim 74, wherein a nucleoside(s) is attached to Z, A, R₃, and/or R₄.

90. The polymer of claim 74, wherein an oligonucleotide(s) is attached to Z, A, R₃, and/or R₄.

91. The polymer of claim 74, wherein a ligand(s) is attached to Z, A, R₃, and/or R₄.that binds to a biological receptor.

92. The polymer of claim 74, wherein a pharmaceutical agent(s) is attached to Z, A, R₃, and/or R₄.

93. The polymer of claim 74, wherein a pharmaceutical agent is attached to Z, A, R₃, and/or R₄ and is at least one selected from the group consisting of antibacterial, anticancer, anti-inflammatory, and antiviral.

94. The polymer of claim 74, wherein a pharmaceutical agent(s) is encapsulated.

95. The polymer of claim 74, wherein camptothecin or a deriviative of campothethcin is encapsulated

96. The polymer of claim 74, wherein a carbohydrate(s) is attached to Z, A, R₃, and/or R₄.

97. The polymer of claim 74, wherein a PET or MRI contrast agent(s) is attached to Z, A, R₃, and/or R₄.

98. The polymer of claim 74, wherein the contrast agent is Gd(DPTA).

99. The polymer of claim 74, wherein an iodated compound for X-ray imagaging is attached to Z, A, R₃, and/or R₄.

100. The polymer of claim 74, wherein the carbohydrate is mannose or sialic acid is attached to the polymer.

101. A polymer of claim 74 wherein which includes a chain which is a polymer or copolymer of a polyester, polyamide, polyether, or polycarbonate of or the polymer in claim 74 in combination with a polyester, polyamide,
polyether, or polycarbonate of:

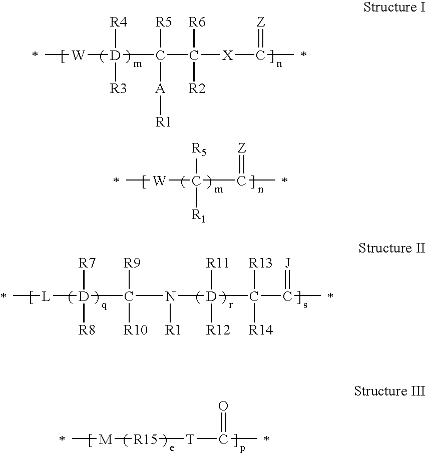

102. A block or random copolymer as in claim 101, which includes at least one terminal or internal crosslinkable group selected from the group consisting of amines, thiols, amides, phosphates, sulphates, hydroxides, alkenes, and alkynes.

103. The polymer of claim 101, wherein X, Y, M is O, S, N—H, N—R, wherin R is —H, CH₂, CR₂ or a chain as defined above, Se or any isoelectronic species of oxygen.

104. The polymer of claim 101, wherein an amino acid(s) is attached to Z, A, R₃, and/or R₄.

105. The polymer of claim 101, wherein a polypeptide(s) is attached to Z, A, R₃, and/or R₄.

106. The polymer of claim 101, wherein an antibody(ies) or single chain antibody(ies) is attached to Z, A, R₃, and/or R₄.

107. The polymer of claim 101, wherein a nucleotide(s) is attached to Z, A, R₃, and/or R₄.

108. The polymer of claim 101, wherein a nucleoside(s) is attached to Z, A, R₃, and/or R₄.

109. The polymer of claim 101, wherein an oligonucleotide(s) is attached to Z, A, R₃, and/or R₄.

110. The polymer of claim 101, wherein a ligand(s) is attached to Z, A, R₃, and/or R₄ that binds to a biological receptor.

111. The polymer of claim 101, wherein a pharmaceutical agent(s) is attached to Z, A, R₃, and/or R₄.

112. The polymer of claim 101, wherein a carbohydrate(s) is attached to Z, A, R₃, and/or R₄.

113. The polymer of claim 101, wherein a PET or MRI contrast agent(s) is attached to Z, A, R₃, and/or R₄.

114. The polymer of claim 101, wherein the contrast agent is Gd(DPTA).

115. The polymer of claim 101, wherein an iodated compound(s) for X-ray imagaging is attached to Z, A, R₃, and/or R₄.

116. The polymer of claim 101, wherein a pharmaceutical agent(s) is attached to Z, A, R₃, and/or R₄ and is at least one selected from the group consisting of antibacterial, anticancer, anti-inflammatory, and antiviral.

117. The polymer of claim 101, wherein the carbohydrate is mannose or sialic acid is covalently attached to the polymer.

118. A surgical procedure which comprises using a photopolymerizable, or chemically crosslinkable, or non-covalenlty crosslinkable polymer or copolymer according to claim 1.

119. The surgical procedure as in claim 118, which is at least one selected from the group consisting of ophthalmic procedures, cardiovascular procedures, plastic surgery procedures, orthopedic procedures, gynecological procedures, ENT procedures, brain procedures, plastic surgery, skin procedures, and cancer treatment.

120. A surgical procedure which comprises using a dendritic polymer or copolymer according to claim 1.

121. The surgical procedure as in claim 120 which is at least one selected from the group consisting of ophthalmic procedures, cardiovascular procedures, plastic surgery procedures, orthopedic procedures, gynecological procedures, ENT procedures, brain procedures, plastic surgery, skin procedures and cancer treatment.

122. The surgical procedure of claim 118 or 120, wherein said dendritic polymer or copolymer is dissolved or suspended in an an aqueous solution wherein the said aqueous solution is selected from water, buffered aqueous media, saline, buffered saline, solutions of amino acids, solutions of sugars, solutions of vitamins, solutions of carbohydrates or combinations of any two or more thereof.

123. The surgical procedure of claims 118 or 120 wherein the supramolecular structure of the dendrimer is a vesicle, micelle, or other such supramolecular structure.

124. The surgical procedure of claims 118 or 120, wherein said dendritic polymer or copolymer is dissolved or suspended in an non-aqueous liquid such as soybean oil, mineral oil, corn oil, rapeseed oil, coconut oil, olive oil, saflower oil, cottonseed oil, aliphatic, cycloaliphatic or aromatic hydrocarbons having 4-30 carbon atoms, aliphatic or aromatic alcohols having 1-30 carbon atoms, aliphatic or aromatic esters having 2-30 carbon atoms, alkyl, aryl or cyclic ethers having 2-30 carbon atoms, alkyl or aryl halides having 1-30 carbon atoms and optionally having more than one halogen substituent, ketones having 3-30 carbon atoms, polyalkylene glycol or combinations of any two or more thereof.

125. The surgical procedure of claims 118 or 120, wherein the supramolecular structure of the dendrimer is in emulsion.

126. The dendritic polymer or copolymer according to claim 1 which optionally contains at least one stereochemical center.

127. The dendritic polymer or copolymer according to claim 1 which is of D or L configuration.

128. The dendritic polymer or copolymer of claim 1, wherein the final dendritic polymer or monomer is chiral or is achiral.

129. The dendritic polymer or copolymer according to claim 1 which optionally contains at least one site where the branching is incomplete.

130. The dendritic polymer or copolymer according to claim 1 made by a convergent or divergent synthesis.
